(19) United States
(12) Patent Application Publication
(10) Pub. No.: US 2025/0241954 A1
(43) Pub. Date: Jul. 31, 2025

(54) GENE-EDITED NATURAL KILLER CELLS

(71) Applicant: CRISPR Therapeutics AG, Zug (CH)

(72) Inventors: Alireza Rezania, South Boston, MA (US); Valentin Sluch, South Boston, MA (US); Meichen Liao, South Boston, MA (US); Viktoriia Kyrychenko, South Boston, MA (US)

(21) Appl. No.: 19/041,857

(22) Filed: Jan. 30, 2025

Related U.S. Application Data
(60) Provisional application No. 63/627,792, filed on Jan. 31, 2024.

(51) Int. Cl. A61K 35/17; A61K 40/15; A61K 40/31; A61K 40/42; A61P 35/00; C07K 14/54; C07K 14/715; C07K 14/725; C12N 5/0783

(52) U.S. Cl. CPC A61K 35/17; A61K 40/15; A61K 40/31; A61K 40/4202; A61P 35/00; C07K 14/5443; C07K 14/7051; C07K 14/7155; C12N 5/0646; A61K 2239/55; C12N 2510/00

(57) ABSTRACT

The present disclosure relates to genetically modified cells (e.g., iPSC, IPS-derived immune cells, e.g., NK or T cells, or NK cells or T cells) comprising a disrupted FAS gene, an insertion of a polynucleotide encoding an IL15/IL15Rα fusion, and an insertion of a polynucleotide encoding a CAR, e.g., an anti-GPR87 CAR. Therapeutic uses of the genetically modified cells to treat cancer (e.g., a lung cancer) are also provided.

### CROSS-REFERENCE TO RELATED APPLICATIONS

This application claims the benefit under 35 U.S.C. § 119(e) of U.S. Provisional Patent Application No. 63/627,792, filed Jan. 31, 2024. The content of this related application is incorporated herein by reference in its entirety for all purposes.

### REFERENCE TO SEQUENCE LISTING

The present application is being filed along with a Sequence Listing in electronic format. The Sequence Listing is provided as a file entitled 80EM-341779-US_SeqList, created Jan. 29, 2025, which is 89 kilobytes in size. The information in the electronic format of the Sequence Listing is incorporated herein by reference in its entirety.

### BACKGROUND

### Field

The present disclosure generally relates to the field of molecular biology and biotechnology, including gene editing.

### Description of the Related Art

Natural Killer (NK) cells are lymphocytes involved in the innate immune response. Due to their function, NK cells are becoming cells of interest for use in the treatment of different diseases such as cancer. Recent success in editing immune cells (e.g., CAR T cells) for enhanced therapeutic ability prompts the use of NK cells in further therapy discoveries. However, the use of NK cells, in particular NK cells expressing a chimeric antigen receptor (CAR), for adoptive cell therapy remains to be challenging. There is a need to improve the efficacy, persistence, cytotoxic activity, biodistribution, immune evasion and tumor targeting of therapeutic NK cells.

### SUMMARY

Disclosed herein includes a genetically modified cell. In some embodiments, the genetically modified cell comprises a disrupted FAS gene, a disrupted B2M gene, an insertion of a polynucleotide encoding a fusion of IL15 and IL15Rα (IL15/IL15Rα) in the disrupted B2M gene, and an insertion of a polynucleotide encoding a CAR, wherein the cell expresses the IL15/IL15Rα fusion protein and the anti-GPR87 CAR, and the cell has disrupted expressions of FAS.

In some embodiments, the CAR is an anti-GPR87 CAR. In some embodiments, the cell comprises a disrupted CIITA gene, and the polynucleotide encoding the CAR is inserted into the disrupted CIITA gene. The genetically modified cell can comprise a disrupted CISH gene. In some embodiments, the genetically modified cell does not comprise a disrupted CISH gene. In some embodiments, the polynucleotide encoding the anti-GPR87 CAR comprises the sequence of SEQ ID NO: 47. In some embodiments, the genetically modified cell does not comprise a genetic modification of a major histocompatibility complex (MHC) gene or a transcriptional regulator gene thereof. In some embodiments, the genetically modified cell does not comprise an insertion of a polynucleotide encoding HLA-E, an insertion of a polynucleotide encoding SERPINB9, or both. In some embodiments, the genetically modified cell does not comprise a disrupted CIITA gene.

The genetically modified cell can be a stem cell. In some embodiments, the stem cell is an induced pluripotent stem cell (iPSC), a hematopoietic stem cell, an embryonic stem cell, or an adult stem cell. In some embodiments, the genetically modified cell is a genome-edited iPSC. The genetically modified cell can be a natural killer (NK) cell obtained from a genome-edited iPSC. The genetically modified cell can be a differentiated cell or a somatic cell. The genetically modified cell can be capable of being differentiated into lineage-restricted progenitor cells or fully differentiated somatic cells. In some embodiments, the genetically modified cell is a natural killer (NK) cell. The NK cell has been differentiated from a genome-edited iPSC, wherein the NK cell comprises the genome edits of the genome-edited iPSC, and wherein the NK cell has not been genome-edited after the differentiation. In some embodiments, the genetically modified cell is capable of cell expansion in the absence of exogenous IL15 in cell culture media.

Provided herein also includes a population of cells comprising one or more genetically modified cells disclosed herein. Provided herein also includes a population of cells comprising lineage-restricted progenitor cells or fully differentiated somatic cells derived from the one or more genetically modified cells disclosed herein. In some embodiments, the lineage-restricted progenitor cells are hematopoietic progenitor cells, mesodermal cells, definitive hemogenic endothelium, definitive hematopoietic stem or progenitor cells, CD34+ cells, multipotent progenitors (MPP), common lymphoid progenitor cells, T cell progenitors, NK cell progenitors, pancreatic endoderm progenitors, pancreatic endocrine progenitors, mesenchymal progenitor cells, muscle progenitor cells, blast cells, or neural progenitor cells; and the fully differentiated somatic cells are pancreatic beta cells, epithelial cells, endodermal cells, macrophages, hepatocytes, adipocytes, kidney cells, blood cells, cardiomyocytes, or immune system cells. The population of cells can comprise NK cells, T cells, B cells, or NKT cells.

The population of cells can comprise human NK cells. In some embodiments, the human NK cells express at least one, two, three, four or five of the markers selected from the group consisting of CD56, NKp44, NKp46, CD94, NKG2A, KIR2DL4, and a CAR; optionally wherein the CAR is detectable by Protein L binding; and further optionally wherein the at least one, two, three, four or five markers are expressed in at least 25%, 30%, 40%, 50%, or 75% of the population of cells. In some embodiments, the population of cells comprising human NK cells has at least one of the following characteristics, or any combination thereof: (i) at least 50% increase in biodistribution to a target tissue; (ii) cytotoxic activity resulting in killing more than 50% of target cells when the population of cells comprising human NK cells are mixed with the target cells at the ratio of 1:1, and (iii) at least 50% increase in cellular viability relative to a population of unmodified human NK cells. In some embodiments, the population of cells comprising human NK cells has at least one of the following characteristics, or any combination thereof: (i) improved persistency, (ii) improved immune evasiveness, (iii) improved cytotoxic activity, (iv) improved antibody-dependent cellular cytotoxicity (ADCC) activity, and (v) improved anti-tumor activity; wherein the characteristics are improved relative to a population of unmodified human NK cells. The population of cells comprising human NK cells, when co-cultured in vitro with a population of cancer cells, are capable of inducing cell death of at least 60%, at least 70%, at least 80%, or at least 90% of the population of cancer cells after about 24 hours of co-culture. The population of cells comprising human NK cells, when co-cultured in vitro with a population of cancer cells, are capable of secreting at least one of interferon gamma (IFNγ), tumor necrosis factor alpha (TNFα), and Granzyme B (GRNB). In some embodiments, the ratio of the human NK cells to cancer cells is 0.1:1 to 4:1.

Provided herein also includes a composition comprising the population of cells described herein. The composition can be for use in treating a subject in need thereof. The composition can be for use in treating cancer in a subject in need thereof. In some embodiments, the cancer is lung cancer, optionally non-small cell lung (NSCLC) or small cell lung cancer (SCLC). The subject can be human.

Provided herein also includes a method for treating a subject in need thereof. In some embodiments, the method comprises (a) obtaining or having obtained the population of cells described herein or obtaining or having obtained the population of cells following differentiation into lineage-restricted progenitor cells or fully differentiated somatic cells; and (b) administering the lineage-restricted progenitor cells or fully differentiated somatic cells to the subject. The lineage-restricted progenitor cells can be hematopoietic progenitor cells, mesodermal cells, definitive hemogenic endothelium, definitive hematopoietic stem or progenitor cells, CD34+ cells, multipotent progenitors (MPP), common lymphoid progenitor cells, T cell progenitors, NK cell progenitors, pancreatic endoderm progenitors, pancreatic endocrine progenitors, mesenchymal progenitor cells, muscle progenitor cells, blast cells, or neural progenitor cells, and the fully differentiated somatic cells are pancreatic beta cells, epithelial cells, endodermal cells, macrophages, hepatocytes, adipocytes, kidney cells, blood cells, cardiomyocytes, or immune system cells. The fully differentiated somatic cells can be NK cells. In some embodiments, the subject has, is suspected of having, or is at risk for a cancer; optionally the subject is human.

In some embodiments, the method comprises administering the NK cells to the subject who has, is suspected of having or is at risk for a cancer, thereby inhibiting the progression of the cancer. The cancer can be a lung cancer, and optionally the NK cells are localized to the site of the cancer following administration. In some embodiments, the method comprises administering about 1×10² to 1×10¹⁰ per gram to the subject; and optionally about 1×10⁶ NK cells per gram to the subject. The NK cells can be administered to the subject more than once; optionally, the NK cells can be administered to the subject at least three times. In some embodiments, the NK cells are administered to the subject in a cycle of at least 7 days. In some embodiments, the NK cells are administered to the subject one, two, or three times in a week.

In some embodiments, inhibiting progression of the cancer comprises inhibition of growth of one or more tumors in the subject and/or reducing the number of cancer cells detected in the subject relative to an untreated subject. In some embodiments, inhibiting progression of the cancer comprises inhibition of growth of one or more tumors in the subject and/or reducing the number of cancer cells detected in the subject relative to the subject prior to administration of the NK cells. In some embodiments, the number of cancer cells detected in the subject increases by no more than 0.5-fold after administration of the NK cells, following one or more cycles of treatment. In some embodiments, the growth of at least one of the one or more tumors in the subject is inhibited by at least about 70% following one or more cycles of treatment. The subject can be tumor-free following one or more cycles of treatment. The NK cells can persist in the subject for at least one week following administration, optionally for at least two weeks following administration. In some embodiments, the number of NK cells detected in the subject decreases by less than 20% one week after administration or by less than 50% two weeks after administration.

### BRIEF DESCRIPTION OF THE DRAWINGS

FIG. 1 shows cell viabilities and biodistribution fluorescence imaging of different engineered iNK cells in comparison to WT NK cells.

FIG. 2A is a graph showing relative radiance intensity percent of different engineered iNK cells in comparison to WT NK cells. FIG. 2B is a graph showing hCD45+/hCD56+ in mice blood with iNK cells with IL15/IL15Rα fusion protein KI with FAS KO.

FIG. 3A presents a schematic for an in vivo protocol to test the therapeutic efficacy of iNK cells comprising anti-GPR87 CAR, IL15/IL15Rα fusion protein KI, CISH KO, and FAS KO.

FIG. 3B shows cell viabilities and biodistribution fluorescence imaging of engineered iNK cells comprising anti-GPR87 CAR, IL15/IL15Rα fusion protein KI, CISH KO, and FAS KO in comparison to control cells. FIG. 3C is a plot showing the radiance intensity of the engineered iNK cells in comparison to the control cells.

FIG. 4A presents a schematic of an in vivo protocol to test the therapeutic efficacy of different engineered iNK cells in comparison to control cells. FIG. 4B shows cell viabilities and biodistribution fluorescence imaging of different engineered iNK cells in comparison to control cells.

FIG. 4C is a plot showing the radiance of different engineered iNK cells. FIG. 4D is a plot showing the percentage of hCD45+/hCD56+ cells in mice blood 2 wks post-iNK injection.

FIG. 5A provides a graph demonstrating L540 cell killing by iNK cells treated with interleukins IL2/IL18 and IL2/IL12/IL18 in comparison to control iNK cells. FIG. 5B is a graph showing the NKG2D expression level of CD30+ iNK cells overexpressing NKG2D (via NKG2D lenti-viral overexpression) with or without IL12 treatment. FIG. 5C is a graph showing the NKG2D expression level of CD30+ iNK cells comprising FAS knock-out and NKG2D overexpression. Lenti-NKG2D Y79 viral construct was expressed at higher level than Lenti-NKG2D Y78 construct, and both construct were expressed in over 95% cells.

FIG. 6A-FIG. 6B are graphs demonstrating L540 cell (FIG. 6A) and Karpas cell (FIG. 6B) killing by iNK cells overexpressing NKG2D.

FIG. 7A-FIG. 7B are graphs demonstrating L428 cell (FIG. 7A) and L540 cell (FIG. 7B) killing by iNK cells comprising IL15/IL15Rα+/SERPINB9+/B2M and CD30CAR+/HLA-E+/CIITA−/FAS− (green line) or IL15/IL15Rα+/SERPINB9+/B2M− and CD30 CAR+/HLA-E+/CIITA−/CISH− (orange line) in comparison to baseline iNK cells containing IL15/IL15Rα+/SerpinB9+/B2M and CD30 CAR+/HLA-E+/CIITA− (red line).

FIG. 8 is a plot showing the in vivo efficacy against L540 cancer line by iNK cells comprising IL15/IL15Rα+/SERPINB9+/B2M and CD30 CAR+/HLA-E+/CIITA−/FAS− (green line) or IL15/IL15Rα+/SERPINB9+/B2M and CD30 CAR+/HLA-E+/CIITA−/FAS KO/NKG2D (purple line) in comparison to baseline iNK cells containing IL15/IL15Rα+/SERPINB9+/B2M and CD30-CAR+/HLA-E+/CIITA− (blue line) and PBNK control cells (orange line).

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein includes an engineered cell. The engineered cell can comprise a disrupted FAS gene, an insertion of a polynucleotide encoding IL15/IL15Rα fusion, and an insertion of a polynucleotide encoding a CAR, wherein the CAR is an anti-GPR87 CAR, wherein the cell expresses the IL15/IL15Rα and the anti-GPR87 CAR, and the cell has disrupted expressions of FAS. Disclosed herein also includes a population of cells comprising one or more engineered cells described herein.

Disclosed herein also includes a method for treating a subject in need thereof. The method can comprise (a) obtaining or having obtained a population of cells described herein or obtaining or having obtained the population of cells following differentiation into lineage-restricted progenitor cells or fully differentiated somatic cells; and (b) administering the lineage-restricted progenitor cells or fully differentiated somatic cells to the subject.

### Definition

As used herein, the term “about” means plus or minus 5% of the provided value.

As used herein, the term “induced pluripotent stem cells” or, iPSCs, means that the stem cells are produced from differentiated adult, neonatal or fetal cells that have been induced or changed, i.e., reprogrammed into cells capable of differentiating into tissues of all three germ or dermal layers: mesoderm, endoderm, and ectoderm. The iPSCs produced do not refer to cells as they are found in nature.

The term “hematopoietic stem and progenitor cells,” “hematopoietic stem cells,” “hematopoietic progenitor cells,” or “hematopoietic precursor cells” refers to cells which are committed to a hematopoietic lineage but are capable of further hematopoietic differentiation and include, multipotent hematopoietic stem cells (hematoblasts), myeloid progenitors, megakaryocyte progenitors, erythrocyte progenitors, and lymphoid progenitors. Hematopoietic stem and progenitor cells (HSCs) are multipotent stem cells that give rise to all the blood cell types including myeloid (monocytes and macrophages, neutrophils, basophils, eosinophils, erythrocytes, megakaryocytes/platelets, dendritic cells), and lymphoid lineages (T cells, B cells, NK cells). The term “definitive hematopoietic stem cell” as used herein, refers to CD34⁺ hematopoietic cells capable of giving rise to both mature myeloid and lymphoid cell types including T cells, NK cells and B cells. Hematopoietic cells also include various subsets of primitive hematopoietic cells that give rise to primitive erythrocytes, megakarocytes and macrophages.

As used herein, the term “NK cell” or “Natural Killer cell” refer to a subset of peripheral blood lymphocytes defined by the expression of CD56 or CD16 and the absence of the T cell receptor (CD3). As used herein, the terms “adaptive NK cell” and “memory NK cell” are interchangeable and refer to a subset of NK cells that are phenotypically CD3⁻ and CD56⁺, expressing at least one of NKG2C and CD57, and optionally, CD16, but lack expression of one or more of the following: PLZF, SYK, FceRy, and EAT-2. In some embodiments, isolated subpopulations of CD56⁺ NK cells comprise expression of CD16, NKG2C, CD57, Natural Killer Group Protein 2D (NKG2D), NCR ligands, NKp30, NKp40, NKp46, activating and inhibitory KIRs, NKG2A and/or DNAM-1.

As used herein, the terms “disruption,” “genetic modification” or “gene-edit” generally refer to a genetic modification wherein a site or region of genomic DNA is altered, e.g., by a deletion or insertion, by any molecular biology method, e.g., methods described herein, e.g., by delivering to a site of genomic DNA an endonuclease and at least one gRNA. Exemplary genetic modifications include insertions, deletions, duplications, inversions, and translocations, and combinations thereof. In some embodiments, a genetic modification is a deletion. In some embodiments, a genetic modification is an insertion. In other embodiments, a genetic modification is an insertion-deletion mutation (or indel), such that the reading frame of the target gene is shifted leading to an altered gene product or no gene product. As used herein, the term “engineered cell” refers to a cell with any disruption, genetic modification, or gene-edit.

As used herein, the term “deletion” which may be used interchangeably with the terms “genetic deletion”, “knock-out”, or “KO”, generally refers to a genetic modification wherein a site or region of genomic DNA is removed by any molecular biology method, e.g., methods described herein, e.g., by delivering to a site of genomic DNA an endonuclease and at least one gRNA. In some embodiments, a deletion involves the removal of all or part of a target gene, e.g., all or part of a FAS gene. In some embodiments, a deletion involves the removal of a transcriptional regulator, e.g., a promoter region, of a target gene. In some embodiments, a deletion involves the removal of all or part of a coding region such that the product normally expressed by the coding region is no longer expressed, is expressed as a truncated form, or expressed at a reduced level. In some embodiments, a deletion leads to a decrease in expression of a gene relative to an unmodified cell. In some embodiments, the decrease in expression can be a reduced level of expression (e.g., express less than 30%, less than 25%, less than 20%, less than 10%, less than 5% of the level of an unmodified cell). In some embodiments, the decrease in expression can be eliminated expression (e.g., no expression or do not express a detectable level of RNA and/or protein). Expression can be measured using any standard RNA-based, protein-based, and/or antibody-based detection method (e.g., RT-PCR, ELISA, flow cytometry, immunocytochemistry, and the like). Detectable levels are defined as being higher that the limit of detection (LOD), which is the lowest concentration that can be measured (detected) with statistical significance by means of a given detection method. As described herein, knockout gene edits are described as “KO” in some cases, e.g., a knockout of the FAS gene can be described as “FAS KO”. In some cases, a gene knockout can be described as “−” with regard to the gene(s) that have been knocked out. For example, a knock-out of the FAS gene can be described as “FAS−”.

As used herein, the term “insertion” which may be used interchangeably with the terms “genetic insertion” or “knock-in”, generally refers to a genetic modification wherein a polynucleotide is introduced or added into a site or region of genomic DNA by any molecular biological method, e.g., methods described herein, e.g., by delivering to a site of genomic DNA an endonuclease and at least one gRNA. In some embodiments, an insertion may occur within or near a site of genomic DNA that has been the site of a prior genetic modification, e.g., a deletion or insertion-deletion mutation. In some embodiments, an insertion occurs at a site of genomic DNA that partially overlaps, completely overlaps, or is contained within a site of a prior genetic modification, e.g., a deletion or insertion-deletion mutation. In some embodiments, an insertion involves the introduction of a polynucleotide that encodes a protein of interest. In some embodiments, an insertion involves the introduction of a polynucleotide that encodes a CAR and/or a fusion protein of IL15 and IL15Rα (i.e., a IL15/IL15Rα fusion protein). In some embodiments, an insertion involves the introduction of an exogenous promoter, e.g., a constitutive promoter, e.g., a CAG promoter. In some embodiments, an insertion involves the introduction of a polynucleotide that encodes a noncoding gene. In general, a polynucleotide to be inserted is flanked by sequences (e.g., homology arms) having substantial sequence homology with genomic DNA at or near the site of insertion. As described herein, knock-in gene edits are described as “KI”, e.g., an IL15/IL15Rα fusion knock-in can be described as “IL15/IL15Rα fusion KI”. In some cases, a gene knock-in can be described as “+” with regard to the gene(s) that have been knocked in, e.g., a knock-in of the IL15/IL15Rα fusion can be described as “IL15/IL15Rα+”.

As used herein, the term “gene editing” (including genomic editing) is a type of genetic engineering in which nucleotide(s)/nucleic acid(s) is/are inserted, deleted, and/or substituted in a DNA sequence, such as in the genome of a targeted cell. Targeted gene editing enables insertion, deletion, and/or substitution at pre-selected sites in the genome of a targeted cell (e.g., in a targeted gene or targeted DNA sequence). When an sequence of an endogenous gene is edited, for example by deletion, insertion or substitution of nucleotide(s)/nucleic acid(s), the endogenous gene comprising the affected sequence can be knocked-out or knocked-down due to the sequence alteration. Therefore, targeted editing can be used to disrupt endogenous gene expression.

As used herein, a “CRISPR-Cas9” system is a naturally-occurring defense mechanism in prokaryotes that has been repurposed as a RNA-guided DNA-targeting platform used for gene editing. It relies on the DNA nuclease Cas9, and two noncoding RNAs-crisprRNA (crRNA) and trans-activating RNA (tracrRNA) to target the cleavage of DNA. crRNA drives sequence recognition and specificity of the CRISPR-Cas9 complex through Watson-Crick base pairing typically with a 20 nucleotide (nt) sequence in the target DNA. The CRISPR-Cas9 complex only binds DNA sequences that contain a sequence match to the first 20 nt of the crRNA, single-guide RNA (sgRNA), if the target sequence is followed by a specific short DNA motif (with the sequence NGG) referred to as a protospacer adjacent motif (PAM). TracrRNA hybridizes with the 3′ end of crRNA to form an RNA-duplex structure that is bound by the Cas9 endonuclease to form the catalytically active CRISPR-Cas9 complex, which can then cleave the target DNA. Once the CRISPR-Cas9 complex is bound to DNA at a target site, two independent nuclease domains within the Cas9 enzyme each cleave one of the DNA strands upstream of the PAM site, leaving a double-strand break (DSB) where both strands of the DNA terminate in a base pair (a blunt end). After binding of CRISPR-Cas9 complex to DNA at a specific target site and formation of the site-specific DSB, the next key step is repair of the DSB. Cells use two main DNA repair pathways to repair the DSB: non-homologous end-joining (NHEJ) and homology-directed repair (HDR). In some embodiments, CRISPR-Cas9 gene editing system comprises an RNA-guided nuclease and one or more guide RNAs targeting one or more target genes.

As used herein, the term “RNA-guided endonuclease” refers to a polypeptide capable of binding a RNA (e.g., a gRNA) to form a complex targeted to a specific DNA sequence (e.g., in a target DNA). A non-limiting example of RNA-guided endonuclease is a Cas polypeptide (e.g., a Cas endonuclease, such as a Cas9 endonuclease). In some embodiments, the RNA-guided endonuclease as described herein is targeted to a specific DNA sequence in a target DNA by an RNA molecule to which it is bound. The RNA molecule can include a sequence that is complementary to and capable of hybridizing with a target sequence within the target DNA, thus allowing for targeting of the bound polypeptide to a specific location within the target DNA.

As used herein, the term “guide RNA” or “gRNA” refers to a site-specific targeting RNA that can bind an RNA-guided endonuclease to form a complex, and direct the activities of the bound RNA-guided endonuclease (such as a Cas endonuclease) to a specific target sequence within a target nucleic acid. The guide RNA can include one or more RNA molecules.

Unless otherwise indicated “nuclease” and “endonuclease” are used interchangeably herein to refer to an enzyme which possesses endonucleolytic catalytic activity for polynucleotide cleavage.

As used herein, the term “Cas endonuclease” or “Cas nuclease” refers to an RNA-guided DNA endonuclease associated with the CRISPR adaptive immunity system.

As used herein, the term “invariable region” of a gRNA refers to the nucleotide sequence of the gRNA that associates with the RNA-guided endonuclease. In some embodiments, the gRNA comprises a crRNA and a transactivating crRNA (tracrRNA), wherein the crRNA and tracrRNA hybridize to each other to form a duplex. In some embodiments, the crRNA comprises 5′ to 3′: a spacer sequence and minimum CRISPR repeat sequence (also referred to as a “crRNA repeat sequence” herein); and the tracrRNA comprises a minimum tracrRNA sequence complementary to the minimum CRISPR repeat sequence (also referred to as a “tracrRNA anti-repeat sequence” herein) and a 3′ tracrRNA sequence. In some embodiments, the invariable region of the gRNA refers to the portion of the crRNA that is the minimum CRISPR repeat sequence and the tracrRNA.

As used herein, the term “target DNA” refers to a DNA that includes a “target site” or “target sequence.” The term “target sequence” is used herein to refer to a nucleic acid sequence present in a target DNA to which a DNA-targeting sequence or segment (also referred to herein as a “spacer”) of a gRNA can hybridize, provided sufficient conditions for hybridization exist. For example, the target sequence 5′-GAGCATATC-3′ within a target DNA is targeted by (or is capable of hybridizing with, or is complementary to) the RNA sequence 5′-GAUAUGCUC-3′. Hybridization between the DNA-targeting sequence or segment of a gRNA and the target sequence can, for example, be based on Watson-Crick base pairing rules, which enables programmability in the DNA-targeting sequence or segment. The DNA-targeting sequence or segment of a gRNA can be designed, for instance, to hybridize with any target sequence.

As used herein, the terms “polynucleotide” and “nucleic acid” are used interchangeably herein and refer to a polymeric form of nucleotides of any length, either ribonucleotides or deoxyribonucleotides. A polynucleotide can be single-, double-, or multi-stranded DNA or RNA, genomic DNA, cDNA, DNA-RNA hybrids/triple helices, or a polymer including purine and pyrimidine bases (e.g., the five biologically occurring bases adenine, guanine, thymine, cytosine and uracil) or other natural, chemically or biochemically modified, non-natural, or derivatized nucleotide bases. In some embodiments, a nucleic acid or polynucleotide can refer to any nucleic acid, whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages.

As used herein, a “secondary structure” of a nucleic acid molecule (e.g., an RNA fragment, or a gRNA) refers to the base pairing interactions within the nucleic acid molecule.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

As used herein, “sequence identity” or “identity” in the context of two nucleic acid or polypeptide sequences makes reference to the nucleotide bases or amino acid residues in the two sequences that are the same when aligned for maximum correspondence over a specified comparison window. When percentage of sequence identity or similarity is used in reference to proteins, it is recognized that residue positions which are not identical often differ by conservative amino acid substitutions, where amino acid residues are substituted with a functionally equivalent residue of the amino acid residues with similar physiochemical properties and therefore do not change the functional properties of the molecule.

A functionally equivalent residue of an amino acid used herein typically can refer to other amino acid residues having physiochemical and stereochemical characteristics substantially similar to the original amino acid. The physiochemical properties include water solubility (hydrophobicity or hydrophilicity), dielectric and electrochemical properties, physiological pH, partial charge of side chains (positive, negative or neutral) and other properties identifiable to a person skilled in the art. The stereochemical characteristics include spatial and conformational arrangement of the amino acids and their chirality. For example, glutamic acid is considered to be a functionally equivalent residue to aspartic acid in the sense of the current disclosure. Tyrosine and tryptophan are considered as functionally equivalent residues to phenylalanine. Arginine and lysine are considered as functionally equivalent residues to histidine.

As used herein, the term “binding” refers to a non-covalent interaction between macromolecules (e.g., between a protein and a nucleic acid). While in a state of non-covalent interaction, the macromolecules are said to be “associated” or “interacting” or “binding” (e.g., when a molecule X is said to interact with a molecule Y, it means that the molecule X binds to molecule Y in a non-covalent manner). Binding interactions can be characterized by a dissociation constant (Kd), for example a Kd of, or a Kd less than, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, 10⁻¹⁴ M, 10⁻¹⁵ M, or a number or a range between any two of these values. Kd can be dependent on environmental conditions, e.g., pH and temperature. “Affinity” refers to the strength of binding, and increased binding affinity is correlated with a lower Kd.

As used herein, the term “hybridizing” or “hybridize” refers to the pairing of substantially complementary or complementary nucleic acid sequences within two different molecules. Pairing can be achieved by any process in which a nucleic acid sequence joins with a substantially or fully complementary sequence through base pairing to form a hybridization complex. “Hybridizing” or “hybridize” can comprise denaturing the molecules to disrupt the intramolecular structure(s) (e.g., secondary structure(s)) in the molecule. In some embodiments, denaturing the molecules comprises heating a solution comprising the molecules to a temperature sufficient to disrupt the intramolecular structures of the molecules. In some instances, denaturing the molecules comprises adjusting the pH of a solution comprising the molecules to a pH sufficient to disrupt the intramolecular structures of the molecules. For purposes of hybridization, two nucleic acid sequences or segments of sequences are “substantially complementary” if at least 80% of their individual bases are complementary to one another. In some embodiments, a splint oligonucleotide sequence is not more than about 50% identical to one of the two polynucleotides (e.g., RNA fragments) to which it is designed to be complementary. The complementary portion of each sequence can be referred to herein as a ‘segment’, and the segments are substantially complementary if they have 80% or greater identity.

The terms “complementarity” and “complementary” mean that a nucleic acid can form hydrogen bond(s) with another nucleic acid based on traditional Watson-Crick base paring rule, that is, adenine (A) pairs with thymine (U) and guanine (G) pairs with cytosine (C). Complementarity can be perfect (e.g., complete complementarity) or imperfect (e.g., partial complementarity). Perfect or complete complementarity indicates that each and every nucleic acid base of one strand is capable of forming hydrogen bonds according to Watson-Crick canonical base pairing with a corresponding base in another, antiparallel nucleic acid sequence. Partial complementarity indicates that only a percentage of the contiguous residues of a nucleic acid sequence can form Watson-Crick base pairing with the same number of contiguous residues in another, antiparallel nucleic acid sequence. In some embodiments, the complementarity can be at least 70%, 80%, 90%, 100% or a number or a range between any two of these values. In some embodiments, the complementarity is perfect, i.e., 100%. For example, the complementary candidate sequence segment is perfectly complementary to the candidate sequence segment, whose sequence can be deducted from the candidate sequence segment using the Watson-Crick base pairing rules.

As used herein, the term “vector” refers to a polynucleotide construct, typically a plasmid or a virus, used to transmit genetic material to a host cell. Vectors can be, for example, viruses, plasmids, cosmids, or phage. A vector as used herein can be composed of either DNA or RNA. In some embodiments, a vector is composed of DNA. An “expression vector” is a vector that is capable of directing the expression of a protein encoded by one or more genes carried by the vector when it is present in the appropriate environment. Vectors are preferably capable of autonomous replication. Typically, an expression vector comprises a transcription promoter, a gene, and a transcription terminator. Gene expression is usually placed under the control of a promoter, and a gene is said to be “operably linked to” the promoter.

As used herein, the terms “transfection” or “infection” refer to the introduction of a nucleic acid into a host cell, such as by contacting the cell with a recombinant MVA virus or a gutless picornaviral particle as described herein.

As used herein, the term “transgene” refers to any nucleotide or DNA sequence that is integrated into one or more chromosomes of a target cell by human intervention. In some embodiment, the transgene comprises a polynucleotide that encodes a protein of interest. The protein-encoding polynucleotide is generally operatively linked to other sequences that are useful for obtaining the desired expression of the gene of interest, such as transcriptional regulatory sequences. In some embodiments, the transgene can additionally comprise a nucleic acid or other molecule(s) that is used to mark the chromosome where it has integrated.

As used herein, the term “prophylaxis,” “prevent,” “preventing,” “prevention,” and grammatical variations thereof as used herein refers the preventive treatment of a subclinical disease-state in a subject, e.g., a mammal (including a human), for reducing the probability of the occurrence of a clinical disease-state. The method can partially or completely delay or preclude the onset or recurrence of a disorder or condition and/or one or more of its attendant symptoms or barring a subject from acquiring or reacquiring a disorder or condition or reducing a subject's risk of acquiring or requiring a disorder or condition or one or more of its attendant symptoms. The subject is selected for preventative therapy based on factors that are known to increase risk of suffering a clinical disease state compared to the general population. “Prophylaxis” therapies can be divided into (a) primary prevention and (b) secondary prevention. Primary prevention is defined as treatment in a subject that has not yet presented with a clinical disease state, whereas secondary prevention is defined as preventing a second occurrence of the same or similar clinical disease state.

As used herein, “treatment” refers to a clinical intervention made in response to a disease, disorder or physiological condition manifested by a patient or to which a patient may be susceptible. The aim of treatment includes, but is not limited to, the alleviation or prevention of symptoms, slowing or stopping the progression or worsening of a disease, disorder, or condition and/or the remission of the disease, disorder or condition. “Treatments” refer to one or both of therapeutic treatment and prophylactic or preventative measures. Subjects in need of treatment include those already affected by a disease or disorder or undesired physiological condition as well as those in which the disease or disorder or undesired physiological condition is to be prevented. A treatment is considered “effective treatment,” if any one or all of the signs or symptoms of, as but one example, levels of functional target are altered in a beneficial manner (e.g., increased by at least 10%), or other clinically accepted symptoms or markers of disease (e.g., cancer) are improved or ameliorated. Efficacy can also be measured by failure of a subject to worsen as assessed by hospitalization or need for medical interventions (e.g., progression of the disease is halted or at least slowed). Methods of measuring these indicators are known to those of skill in the art and/or described herein. Treatment includes any treatment of a disease in subject and includes: (1) inhibiting the disease, e.g., arresting, or slowing the progression of symptoms; or (2) relieving the disease, e.g., causing regression of symptoms; and (3) preventing or reducing the likelihood of the development of symptoms.

As used herein, the terms “effective amount” or “pharmaceutically effective amount” or “therapeutically effective amount” refer to an amount sufficient to effect beneficial or desirable biological and/or clinical results. An effective amount also includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

The term “pharmaceutically acceptable excipient” as used herein refers to any suitable substance that provides a pharmaceutically acceptable carrier, additive or diluent for administration of a compound(s) of interest to a subject. Pharmaceutically acceptable excipient can encompass substances referred to as pharmaceutically acceptable diluents, pharmaceutically acceptable additives, and pharmaceutically acceptable carriers.

As used herein, a “subject” refers to an animal for whom a diagnosis, treatment, or therapy is desired. In some embodiments, the subject is a mammal. “Mammal,” as used herein, refers to an individual belonging to the class Mammalia and includes, but not limited to, humans, domestic and farm animals, zoo animals, sport and pet animals. Non-limiting examples of mammals include mice; rats; rabbits; guinea pigs; dogs; cats; sheep; goats; cows; horses; primates, such as monkeys, chimpanzees and apes, and, in particular, humans. In some embodiments, the mammal is a primate. In some embodiments, the mammal is a human. In some embodiments, the mammal is not a human.

The wording “associated with” as used herein with reference to two items indicates a relation between the two items such that the occurrence of a first item is accompanied by the occurrence of the second item, which includes but is not limited to a cause-effect relation and sign/symptoms-disease relation.

### Gene Editing

Provided herein include engineered cells (e.g., iPSC, IPS-derived NK, or NK cell) and compositions and methods for producing the engineered cells. The compositions and methods described herein enable the engineered cells to evade immune response and/or increase their survival or viability following engraftment into a subject. In some embodiments, the gene-edited cells can evade immune response and/or survive at higher success rates than an unmodified cell.

As used herein, the term “engineered cell” generally refers to a genetically modified cell that demonstrates increased survival and biodistribution after transplantation and/or is less susceptible to allogeneic rejection during a cellular transplant, relative to an unmodified cell. In some embodiments, a genetically modified cell as described herein is an engineered cell. In some embodiments, the engineered cell has increased immune evasion, prolonged cell survival, improved biodistribution and in vivo therapeutic efficacy compared to an unmodified cell. In some embodiments, the engineered cell has (i) improved persistency, (ii) improved immune evasiveness, (iii) improved cytotoxic activity, (iv) improved ADCC activity, (v) increased cell survival, and/or (vi) improved anti-tumor activity compared to an unmodified cell. In some embodiments, an engineered cell may be a stem cell. In some embodiments, an engineered cell may be an embryonic stem cell (ESC), an adult stem cell (ASC), an induced pluripotent stem cell (iPSC), or a hematopoietic stem or progenitor cell (HSPC). In some embodiments, an engineered cell may be a differentiated cell. In some embodiments, an engineered cell may be a somatic cell (e.g., immune system cells). In some embodiments, an engineered cell is administered to a subject. In some embodiments, an engineered cell is administered to a subject who has, is suspected of having, or is at risk for a disease (e.g., a cancer such as lung cancer). In some embodiments, the engineered cell is capable of being differentiated into lineage-restricted progenitor cells or fully differentiated somatic cells. In some embodiments, the lineage-restricted progenitor cells are pancreatic endoderm progenitors, pancreatic endocrine progenitors, mesenchymal progenitor cells, muscle progenitor cells, blast cells, or neural progenitor cells. In some embodiments, the fully differentiated somatic cells are endocrine secretory cells such as pancreatic beta cells, epithelial cells, endodermal cells, macrophages, hepatocytes, adipocytes, kidney cells, blood cells, or immune system cells.

Any cells described herein can be gene-edited using any of the gene-editing methods described herein (e.g., using CRISPR/Cas gene editing to insert or delete one or more nucleotides). In some embodiments, a disrupted gene is a gene that does not encode functional protein. In some embodiments, a cell that comprises a disrupted gene does not express (e.g., at the cell surface) a detectable level (e.g., by antibody, e.g., by flow cytometry) of the protein encoded by the gene. A cell that does not express a detectable level of the protein may be referred to as a knockout cell.

In some embodiments, the cells described herein are gene-edited to disrupt one or more of the genes described herein. For example, the cells can be gene-edited to disrupt a FAS gene, a CISH gene, or both.

### FAS Gene Edits

In some embodiments, the genome of any cell described herein is modified to disrupt a Fas cell surface death receptor (FAS) gene (NCBI Gene ID: 355). FAS is a member of the TNF-receptor superfamily and contributes to the regulation of programmed cell death. In some embodiments, the disrupted FAS can reduce activation-induced cell death (AICD), resist apoptosis, and/or increase tumor killing. In some embodiments, an iPSC comprises a disrupted FAS gene (e.g., FAS KO). In some embodiments, an NK cell comprises a disrupted FAS gene (e.g., FAS KO).

In some embodiments, gRNAs targeting the FAS genomic region create Indels in the FAS gene disrupting expression of the mRNA or protein. In some embodiments, the gRNA targets a site within the FAS gene. In some embodiments, the FAS gRNA targets a sequence comprising SEQ ID NOS: 9-12, 19, 20, and 26. In some embodiments, a gRNA targeting the FAS gene comprises a spacer sequence corresponding to a sequence comprising any one of SEQ ID NOS: 9-12, 19, 20, and 26.

In some embodiments, at least 50% of the engineered cells of a population of cells does not express a detectable level of FAS protein. For example, at least 55%, at least 60%, at least 70%, at least 75%, at least 80%, at least 85%, at least 90%, or at least 95% of the engineered cells of a population may not express a detectable level of FAS surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered cells of a population does not express a detectable level of FAS protein.

In some embodiments, less than 50% of the engineered cells of a population of cells express a detectable level of FAS protein. In some embodiments, less than 30% of the engineered cells of a population of cells express a detectable level of FAS protein. For example, less than 50%, less than 30%, less than 25%, less than 20%, less than 15%, less than 10%, or less than 5% of the engineered cells of a population of cells express a detectable level of FAS protein. In some embodiments, 40%-30%, 40%-20%, 40%-10%, 40%-5%, 30%-20%, 30%-10%, 30%-5%, 20%-10%, 20%-5%, or 10%-5% of the engineered cells of a population of cells express a detectable level of FAS protein.

### CISH Gene Edits

In some embodiments, the genome of any cell described herein is modified to disrupt a cytokine inducible SH2 containing protein (CISH, also called CIS) gene (NCBI Gene ID: 1154). CISH is a transcriptional co-activator that controls expression of HLA class II genes. In some embodiments, the disrupted CISH can increase iNK sensitivity to cytokines, improve iNK persistence, and/or increase tumor killing. In some embodiments, an iPSC comprises a disrupted CISH gene. In some embodiments, an NK cell comprises a disrupted CISH gene.

In some embodiments, gRNAs targeting the CISH genomic region create Indels in the CISH gene disrupting expression of the mRNA or protein. In some embodiments, the gRNA targets a site within the CISH gene. In some embodiments, the CISH gRNA targets a sequence comprising SEQ ID NOS: 27-38. In some embodiments, a gRNA targeting the CISH gene comprises a spacer sequence corresponding to a sequence comprising any one of SEQ ID NOS: 27-38.

In some embodiments, at least 50% of the engineered cells of a population of cells does not express a detectable level of CISH protein. For example, at least 55%, at least 60%, at least 70%, at least 75%, at least 80%, at least 85%, at least 90%, or at least 95% of the engineered cells of a population may not express a detectable level of CISH surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered cells of a population does not express a detectable level of CISH protein.

In some embodiments, less than 50% of the engineered cells of a population of cells express a detectable level of CISH protein. In some embodiments, less than 30% of the engineered cells of a population of cells express a detectable level of CISH protein. For example, less than 50%, less than 30%, less than 25%, less than 20%, less than 15%, less than 10%, or less than 5% of the engineered cells of a population of cells express a detectable level of CISH surface protein. In some embodiments, 40%-30%, 40%-20%, 40%-10%, 40%-5%, 30%-20%, 30%-10%, 30%-5%, 20%-10%, 20%-5%, or 10%-5% of the engineered cells of a population of cells express a detectable level of CISH protein.

### Edits to Knock-in IL15 and IL15Rα

In some embodiments, the cells described herein are gene-edited to insert a polynucleotide encoding, such as a polynucleotide encoding IL15, IL15Rα, and/or a fusion protein of interleukin-15 (IL15) and interleukin-15 receptor alpha (IL15Rα) (“IL15/IL15Rα” or “IL15/IL15Rα fusion”). In some embodiments, the genome of any cell described herein comprises an insertion of polynucleotide encoding IL15, IL15Rα, and/or a fusion protein of IL15 and IL15Rα (“IL15/IL15Rα”). IL15 is a cytokine that functions in regulating NK cell proliferation and activation, and is encoded by IL15 gene (MCBI Gene ID: 3600). IL15Rα (also called IR15α) is the receptor that binds IL15, and is encoded by IL15Rα gene (MCBI Gene ID: 16169). In some embodiments, the genome of a cell described herein comprises an insertion of a polynucleotide encoding IL15. In some embodiments, the genome of a cell described herein comprises an insertion of a polynucleotide encoding a fusion protein of IL15 and IL15Rα. In some embodiments, the genome of a cell described herein comprises an insertion of a polynucleotide encoding IL15 and does not comprise an insertion of a polynucleotide encoding IL15Rα. The insertion of the polynucleotide encoding IL15 can lead to increased iNK persistence and prolonged survival of the engineered cell.

In some embodiments, a cell has insertion of a polynucleotide encoding IL15, and the polynucleotide comprises or consists of SEQ ID NO: 13. In some embodiments, the IL15 polynucleotide has at least 90%, 91%, 92%, 93%, 94%, 95%, 96%, 97%, 98%, or 99% identity to SEQ ID NO: 13. In some embodiments, a cell has insertion of a polynucleotide encoding IL15Rα, and the polynucleotide comprises or consists of SEQ ID NO: 14. In some embodiments, a cell has insertion of a polynucleotide encoding a fusion protein of IL15 and IL15Rα (“IL15/IL15Rα”). In some embodiments, the fusion sequence is as described in Hurton et al. (2016) Proc Natl Acad Sci USA.; 113(48):E7788-E7797. doi: 10.1073/pnas.1610544113, which is incorporated herein in its entirety. In some embodiments, the polynucleotide encoding IL15/IL15Rα comprises or consists of SEQ ID NO: 22 (which consists of SEQ ID NOS: 13-14). In some embodiments, the IL15 IL15Rα polynucleotide has at least 90%, 91%, 92%, 93%, 94%, 95%, 96%, 97%, 98%, or 99% identity to SEQ ID NO: 22. In some embodiments, the IL15/IL15Rα fusion has the amino acid sequence of SEQ ID NO: 43.

In some embodiments, IL15 and IL15Rα are co-expressed. In some embodiments, a self-cleaving peptide is used to co-express IL15 and IL15Rα. In some embodiments, the self-cleaving peptide is selected from, but not limited to, P2A (derived from porcine teschovirus-1 2A), E2A (derived from equine rhinitis A virus), F2A (derived from foot-and-mouth disease virus 18), and T2A (derived from thosea asigna virus 2A). In some embodiments, the self-cleaving peptide is derived from P2A. In some embodiments, a cell has insertion of a polynucleotide encoding IL15, P2A, IL15Rα (IL15-P2A-IL15Rα). In some embodiments, an iPSC comprises a knock-in of the IL15-P2A-IL15Rα polynucleotide. In some embodiments, an NK cell comprises a knock-in of the IL15-P2A-IL15Rα polynucleotide.

In some embodiments, at least 50% of the engineered cells of a population of cells express a detectable level of any IL15, IL15Rα, and/or IL15/IL15Rα described herein. For example, at least 55%, at least 60%, at least 70%, at least 75%, at least 80%, at least 85%, at least 90%, or at least 95% of the engineered cells of a population express a detectable level of IL15, IL15Rα, and/or IL15/IL15Rα. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered cells of a population expresses a detectable level of IL15, IL15Rα, and/or IL15/IL15Rα.

In some embodiments, less than 50% of the engineered cells of a population of cells do not express a detectable level of IL15, IL15Rα, and/or IL15/IL15Rα. In some embodiments, less than 30% of the engineered cells of a population of cells do not express a detectable level of IL15, IL15Rα, and/or IL15/IL15Rα. For example, less than 50%, less than 30%, less than 25%, less than 20%, less than 15%, less than 10%, or less than 5% of the engineered cells of a population of cells do not express a detectable level of IL15, IL15Rα, and/or IL15/IL15Rα. In some embodiments, 40%-30%, 40%-20%, 40%-10%, 40%-5%, 30%-20%, 30%-10%, 30%-5%, 20%-10%, 20%-5%, or 10%-5% of the engineered cells of a population of cells do not express a detectable level of IL15, IL15Rα, and/or IL15/IL15Rα.

In some embodiments, any of the IL15, IL15Rα, and/or IL15/IL15Rα polynucleotides described herein are inserted into any safe-harbor locus described herein. In some embodiments, any of the IL15, IL15Rα, and/or IL15/IL15Rα polynucleotides described herein are inserted into any B2M gene locus described herein.

### Edits to Knock-In Chimeric Antigen Receptors

A chimeric antigen receptor (CAR) refers to an artificial immune cell receptor that is engineered to recognize and bind to an antigen expressed by tumor cells. CARs can be inserted into any cells described herein. CARs are a chimera of a signaling domain of the T-cell receptor (TCR) complex and an antigen-recognizing domain (e.g., a single chain fragment (scFv) of an antibody or other antibody fragment) (Enblad et al., Human Gene Therapy. 2015; 26(8):498-505). CARs have the ability to redirect cell specificity and reactivity toward a selected target in a non-MHC-restricted manner. The non-MHC-restricted antigen recognition gives cells expressing CARs the ability to recognize an antigen independent of antigen processing, thus bypassing a major mechanism of tumor escape. CARs are often referenced to by the antigen they bind. For example, a “CD30 CAR”, “CD19 CAR”, a “GPC3 CAR”, “GPR87 CAR”, “CD70 CAR”, a “CD33 CAR” and a “BCMA CAR” are CARs comprising antigen binding domains that specifically bind to CD30, CD19, GPC, GPR87 CD70, CD33 or BCMA, respectively. Accordingly, such terms are interchangeable with anti-CD30 CAR, anti-CD19 CAR, anti-GPC3 CAR, anti-GPR87 CAR, anti-CD70 CAR, anti-CD33 CAR and anti-BCMA CAR. It will be understood by those of ordinary skill in the art that a CAR that specifically binds an antigen can be referred to with either terminology.

In some embodiments, any iPSC described herein expresses a CAR. In some embodiments, any NK cell described herein expresses a CAR. In some embodiments, any HSPC described herein expresses a CAR. There are four generations of CARs, each of which contains different components. First generation CARs join an antibody-derived scFv to the CD3zeta (ζ or z) intracellular signaling domain of the T-cell receptor through hinge and transmembrane domains. Second generation CARs incorporate an additional domain, e.g., CD28, 4-1BB (41BB), or ICOS, to supply a costimulatory signal. Third-generation CARs contain two costimulatory domains fused with the TCR CD3ζ chain. Third-generation costimulatory domains may include, e.g., a combination of CD3ζ, CD27, CD28, 4-1BB, ICOS, or OX40. Fourth-generation CARs include immune stimulatory cytokines to improve cell persistence and expansion. Cytokines for fourth-generation CARS include individually or in combination any of IL-7, IL-12, IL-15, IL-18, or IL-23. CARs, in some embodiments, contain an ectodomain, commonly derived from a single chain variable fragment (scFv), a hinge, a transmembrane domain, and an endodomain with one (first generation), two (second generation), or three (third generation) signaling domains derived from CD3Z and/or co-stimulatory molecules (Maude et al., Blood. 2015; 125(26):4017-4023; Kakarla and Gottschalk, Cancer J. 2014; 20(2):151-155).

CARs typically differ in their functional properties. The CD3ζ signaling domain of the T-cell receptor, when engaged, will activate and induce proliferation of T-cells but can lead to anergy (a lack of reaction by the body's defense mechanisms, resulting in direct induction of peripheral lymphocyte tolerance). Lymphocytes are considered anergic when they fail to respond to a specific antigen. The addition of a costimulatory domain in second-generation CARs improved replicative capacity and persistence of modified T-cells. Similar antitumor effects are observed in vitro with CD28 or 4-1BB CARs, but preclinical in vivo studies suggest that 4-1BB CARs may produce superior proliferation and/or persistence. Clinical trials suggest that both of these second-generation CARs are capable of inducing substantial T-cell proliferation in vivo, but CARs containing the 4-1BB costimulatory domain appear to persist longer. Third generation CARs combine multiple signaling domains (costimulatory) to augment potency.

In some embodiments, a chimeric antigen receptor is a first-generation CAR. In other embodiments, a chimeric antigen receptor is a second-generation CAR. In yet other embodiments, a chimeric antigen receptor is a third generation CAR. In some embodiments, a chimeric antigen receptor is a fourth-generation CAR.

A CAR, in some embodiments, comprises an extracellular (ecto) domain comprising an antigen binding domain (e.g., an antibody, such as an scFv), a transmembrane domain, and a cytoplasmic (endo) domain.

### Ectodomain of CARs

The ectodomain is the region of the CAR that is exposed to the extracellular fluid and, in some embodiments, includes an antigen binding domain, and optionally a signal peptide, a spacer domain, and/or a hinge domain. In some embodiments, the antigen binding domain is a single-chain variable fragment (scFv) that includes the VL and VH of immunoglobulins connected with a short linker peptide. The linker, in some embodiments, includes hydrophilic residues with stretches of glycine and serine for flexibility as well as stretches of glutamate and lysine for added solubility. A single-chain variable fragment (scFv) is not actually a fragment of an antibody, but instead is a fusion protein of the variable regions of the heavy (VH) and light chains (VL) of immunoglobulins, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and the introduction of the linker. In some embodiments, the scFv of the present disclosure is humanized. In other embodiments, the scFv is fully human. In yet other embodiments, the scFv is a chimera (e.g., of mouse and human sequence).

In some embodiments, the scFv is an anti-BCMA scFv (binds specifically to BCMA). The scFv can be an anti-CD30 scFv (binds specifically to CD30). In some embodiments, anti-CD30 scFv may comprise variable domains from mouse monoclonal AC10 (e.g., Brentuximab). In other embodiments, anti-CD30 scFv may comprise variable domains from human 5F11 antibody (U.S. Pat. No. 7,387,776).

In some embodiments, the scFv is an anti-CD19 scFv (binds specifically to CD19). In some embodiments, the scFv is an anti-CD70 scFv (binds specifically to CD70). In some embodiments, the scFv is an anti-CD33 scFv (binds specifically to CD33). In some embodiments, the scFv is an anti-GPC3 scFv (binds specifically to GPC3). In some embodiments, the scFv is an anti-GPR87 scFv (binds specifically to GPR87). In some embodiments, the scFv is an anti-A33 scFv (binds specifically to A33). Other scFv proteins can be used. The ectodomain of the CAR can recognize more than one antigen, e.g., the CAR can recognize and bind CD19, CD20, and BCMA.

In some embodiments, the scFv is an anti-GPR87 scFv (binds specifically to GPR87). Non-limiting examples of an anti-GPR87 CAR that may be used as provided herein may include the amino acid sequence of SEQ ID NO: 48. In some embodiments, an anti-GPR87 CAR may comprise the nucleotide sequence of SEQ ID NO: 47. In some embodiments, the anti-GPR87 polynucleotide has at least 90%, 91%, 92%, 93%, 94%, 95%, 96%, 97%, 98%, or 99% identity to SEQ ID NO: 47. In some embodiments, the anti-GPR87 CAR amino acid sequence has at least 90%, 91%, 92%, 93%, 94%, 95%, 96%, 97%, 98%, or 99% identity to SEQ ID NO: 48.

In some embodiments, the ectodomain is the region of the CAR that is exposed to the extracellular fluid and, in some embodiments, includes a NKG2D receptor, and optionally a signal peptide, a spacer domain, and/or a hinge domain. The signal peptide can enhance the antigen specificity of CAR binding. Signal peptides can be derived from antibodies, such as, but not limited to, CD8, as well as epitope tags such as, but not limited to, GST or FLAG. Examples of signal peptides include MLLLVTSLLLCELPHPAFLLIP (SEQ ID NO: 76) and MALPVTALLLPLALLLHAARP (SEQ ID NO: 77). Other signal peptides may be used. In some embodiments, the NKG2D receptor is overexpressed (e.g., by 20%, 30%, 40%, 50%, 60%, 70%, 80%, 90%, 100%, 200%, 300%, 400%, 500%, 600%, 700%, 800%, 900%, 1000% or higher) in the engineered cells described herein.

A spacer domain or hinge domain can be located between an extracellular domain (comprising the antigen binding domain) and a transmembrane domain of a CAR, or between a cytoplasmic domain and a transmembrane domain of the CAR. A spacer domain is any oligopeptide or polypeptide that functions to link the transmembrane domain to the extracellular domain and/or the cytoplasmic domain in the polypeptide chain. A hinge domain is an oligopeptide or polypeptide that functions to provide flexibility to the CAR, or domains thereof, or to prevent steric hindrance of the CAR, or domains thereof. In some embodiments, a spacer domain or a hinge domain may comprise up to 300 amino acids (e.g., 10 to 100 amino acids, or 5 to 20 amino acids). In some embodiments, one or more spacer domain(s) may be included in other regions of a CAR. In some embodiments, the hinge domain is a CD8 hinge domain. Other hinge domains may be used.

### Transmembrane Domain of CARs

The transmembrane domain is a hydrophobic alpha helix that spans the membrane. The transmembrane domain provides stability of the CAR. In some embodiments, the transmembrane domain of the CAR is a CD8 transmembrane domain. In some embodiments, the transmembrane domain is a CD28 transmembrane domain. In yet other embodiments, the transmembrane domain is a chimera of a CD8 and CD28 transmembrane domain. In some embodiments, the transmembrane domain is a CD8a transmembrane domain: FVPVFLPAKPTTTPAPRPPTPAPTIASQPLSLRPEACRPAAGGAVHTRGLDFACDIYIWAPLA GTCGVLLLSLVITLYCNHRNR (SEQ ID NO: 78). In some embodiments, the transmembrane domain is a CD8a transmembrane domain comprising the amino acid sequence: IYIWAPLAGTCGVLLLSLVITLY (SEQ ID NO: 79). In some embodiments, the transmembrane domain is a CD8 transmembrane domain comprising the amino acid sequence SAAAFVPVFLPAKPTTTPAPRPPTPAPTIASQPLSLRPEACRPAAGGAVHTRGLDFACDIYIW APLAGTCGVLLLSLVITLYCNHRNR (SEQ ID NO: 80). Other transmembrane domains can be used.

In some embodiments, the transmembrane domain is selected from transmembrane domains of: NKG2D, FcYRIIIa, NKp44, NKp30, NKp46, actKIR, NKG2C, CD8a, CD28, and IL15Rb. In some embodiments, the transmembrane domain is an NKG2D transmembrane domain. In some embodiments, a CD28 transmembrane domain is used.

### Endodomain of CARs

The endodomain is the functional end of the receptor. Following antigen recognition, receptors cluster and a signal is transmitted to the cell. The most commonly used endodomain component is CD3ζ (CD3-zeta), which contains three (3) immunoreceptor tyrosine-based activation motif (ITAM)s. This transmits an activation signal to the T cell after the antigen is bound. In many cases, CD3-zeta may not provide a fully competent activation signal and, thus, a co-stimulatory signaling is used. For example, CD28 and/or 4-1BB may be used with CD3-zeta (CD3ζ) to transmit a proliferative/survival signal. Thus, in some embodiments, the co-stimulatory molecule of a CAR as provided herein is a CD28 co-stimulatory molecule. In other embodiments, the co-stimulatory molecule is a 4-1BB co-stimulatory molecule. In some embodiments, a CAR includes CD3-zeta and CD28. In some embodiments, a CAR includes CD3-zeta and 4-1BB. In still other embodiments, a CAR includes CD3ζ, CD28, and 4-1BB.

In some embodiments, any of the CARs described herein have one, two or more intracellular signaling domains from, e.g., CD137/41 BB, DNAM-1, NKrdO, 2B4, NTBA, CRACC, CD2, CD27, one or more integrins (e.g., ITGB1, ITGB2, or ITGB3), IL-15R, IL-18R, IL-12R, IL-21 R, or IRE1a (e.g., any combination of signaling domains from two or more of these molecules).

Natural Killer cells express a number of transmembrane adapters providing them with signal enhancement. In some embodiments, the intracellular signaling domain of any CAR described herein comprises a transmembrane adapter. In some embodiments, the transmembrane adapter is a transmembrane adaptor from one or more of: FceR1 y, CD3ζ, DAP 12, and DAP 10.

In some embodiments, a CARs described herein have one of more co-stimulatory domains. In some embodiments, a 2B4 co-stimulatory domain is used. In some embodiments, a CD3ζ intracellular signaling domain is used. In some embodiments, a DAP10 or DAP12 co-stimulatory domains are used with a CD3ζ intracellular signaling domain. In some embodiments, a DAP10 co-stimulatory signaling domain is used with an NKG2D transmembrane domain. In some embodiments, the transmembrane domain is from NKG2D, and the endodomain is from DAP10 and CD3ζ (e.g., as described in Chang Y H et al. Caner Res. 2013. 73(6):1777-86). In some embodiments, the CAR comprises an NKG2D transmembrane domain fused to 4-1BB and DAP10 signaling and/or co-stimulatory domains (e.g., as described in Guo C. et al. Mol Immunol. 2019. 114:108-113). In some embodiments, the CAR comprises a co-stimulatory domain from 2B4. In some embodiments, the CAR comprises a CD8 transmembrane domain and 4-1BB-CD3ζ signaling domains (e.g., as in a construct as described by Imai C, et al. Blood. 2005, 106(1). 376-383).

In some embodiments, the CAR has a CD8 transmembrane domain, a 4-1BB intracellular domain, and a CD3ζ signaling domain. In some embodiments, the CAR has a CD28 transmembrane domain, a CD28 intracellular domain, and a CD3ζ signaling domain. In some embodiments, the CAR has a DAP12 transmembrane and intracellular domains. In some embodiments, the CAR has a 2B4 transmembrane and intracellular domains and a CD3ζ signaling domain. In some embodiments, the CAR has a CD8 transmembrane domain, a 2B4 intracellular domain, and a CD3ζ signaling domain. In some embodiments, the CAR has a CD28 transmembrane and intracellular domains, a 4-1BB intracellular domain, and a CD3ζ signaling domain. In some embodiments, the CAR has a CD16 transmembrane domain, a 2B4 intracellular domain, and a CD3ζ signaling domain. In some embodiments, the CAR has a NKp44 transmembrane domain, a DAP10 intracellular domain, and a CD3ζ signaling domain. In some embodiments, the CAR has a NKp46 transmembrane domain, a2B4 intracellular domain, and a CD3ζ signaling domain. In some embodiments, the CAR has a NKG2D transmembrane domain, a 4-1BB intracellular domain, and a CD3ζ signaling domain. In some embodiments, the CAR has a NKG2D transmembrane domain, a 4-1BB in intracellular domain, and a CD3ζ signaling domain. In some embodiments, the CAR has an NKG2D transmembrane domain, a DAP12 intracellular domain, a 2B4 intracellular domain, and a CD3ζ signaling domain. In some embodiments, the CAR has an NKG2D transmembrane domain, a DAP10 intracellular domain, a 2B4 intracellular domain, and a CD3ζ signaling domain. In some embodiments, the CAR has an NKG2D transmembrane domain, a 4-1BB intracellular domain, a 2B4 intracellular domain, and a CD3ζ signaling domain. In some embodiments, the CAR has an NKG2D transmembrane domain and a CD3ζ signaling domain.

### Multi-Gene Editing

In some embodiments, the engineered cells disclosed herein include more than one gene edit, for example, in more than one gene. In some embodiments, two, three, four, five, six or more genes are edited. In some embodiments, the gene-edit is an insertion (KI). In some embodiments, the gene-edit is a disruption (KO). In some embodiments, the combination of two or more gene edits described herein is a combination of KI and KO. In some embodiments, the gene-edits are any combination of one, two, three, four, five, six or more of the gene-edits selected from: B2M KO, IL15 KI, IL15Rα KI, an IL15/IL15Rα fusion protein KI, anti-GPC3 CAR KI, anti-GPR87 CAR KI, CD16 KI, CD64 KI, NKG2D CAR KI, CISH KO, FAS KO, CD38 KO, FLI1 KO, TGFBR1 KO, TGFBR2 KO, ZEB1 KO, ADORA2A/ADORA2B KO, ADAM17 KO, anti-BCMA CAR KI, anti-CD30 CAR KI, REGNASE-1 KO, TIGIT KO, PD-1 KO, NKG2A KO, CD70 KO, ALK4 KO (e.g., a conditional KO), anti-A33 CAR KI, anti-CD70 CAR KI, anti-CD19 CAR KI, anti-CD33 CAR KI, anti-CD19-CD20-BCMA CAR KI, anti-NKp30 CAR KI, anti-CD73 CAR KI, and anti-SLC7A11(xCT) CAR KI.

In some embodiments, the editing of two or more genes is simultaneous, such as in the same method step. In some embodiments, the editing of two or more genes is sequential, such as in two or more separate steps.

In some embodiments, the engineered cells comprise: a disrupted FAS gene; an insertion of a polynucleotide encoding IL15/IL15Rα fusion protein; and an insertion of a polynucleotide encoding a CAR, wherein the cell expresses the IL15/IL15Rα fusion protein and the CAR, and the cell has disrupted expressions of FAS. In some embodiments, IL15/IL15Rα fusion KI sequence (e.g., a SERPINB9-P2A-IL15/IL15Rα fusion KI sequence) is inserted into a B2M KO. In some embodiments, the CAR is an anti-GPR87 CAR. In some embodiments, the CAR KI sequence (e.g., a CAR-P2A-HLA-E KI sequence) is inserted into CIITA KO.

In some embodiments, a polynucleotide described herein is linked to a promoter, for example an exogenous promoter. In some embodiments, the promoter is selected from but not limited to CAGGS, CMV, EFla, PGK, CAG, UBC, or other constitutive, inducible, temporal-, tissue-, and cell type-specific promoter.

In some embodiments, the engineered cell can comprise a disrupted CISH gene, and therefore disrupted expression of CISH. In some embodiments, the engineered cell does not comprise a disrupted CISH gene. In some embodiments, the engineered cells described herein do not comprise any additional disrupted gene edit other than FAS gene.

In some embodiments, the at least one polynucleotide encoding at least one tolerogenic factor is inserted into a safe harbor locus, e.g., the AAVS1 locus. In some embodiments, a safe harbor locus for inserting any gene described herein is selected from, but not limited to AAVS1 (PPP1 R12C), ALB, Angpt31, ApoC3, ASGR2, CCR5, FIX (F9), G6PC, Gys2, HGD, Lp(a), Pcsk9, Serpinal, TF, and TTR.

In some embodiments, the engineered cells do not comprise an insertion of a polynucleotide encoding HLA-E. HLA-E is a heterodimer class I molecule. HLA-E primarily functions as a ligand for the NK cell inhibitory receptor KLRD1-KLRC1. HLA-E enables NK cells to monitor other MHC class I molecule expression and to tolerate self-expression. In some embodiments, the engineered cells described herein (e.g., NK cell) does not comprise a knock-in HLA-E gene.

In some embodiments, the engineered cells do not comprise an insertion of a polynucleotide encoding SERPINB9. SERPINB9 is a member of a large family of apoptosis inhibitors that mainly function by targeting intermediate proteases (e.g., covalently bind a protease in 1:1 complex, thereby inhibiting the protease). In some embodiments, an engineered cells described herein (e.g., a NK cell) does not comprise a knock-in SERPINB9 gene.

In some embodiments, the engineered cells do not comprise an insertion of a polynucleotide encoding HLA-E, an insertion of a polynucleotide encoding SERPINB9, or both.

In some embodiments, the genome-engineered cells comprise introduced or increased expression in IL15 and/or CAR. The genome-engineered cells can comprise integrated or non-integrated exogenous polynucleotide encoding IL15. The genome-engineered cells can comprise integrated or non-integrated exogenous polynucleotide encoding one or more of any of the CARs disclosed herein (e.g., anti-GPR87 CAR). In some embodiments, said introduced expression is an increased expression from either non-expressed or lowly expressed genes comprised in said cells. In some embodiments, the non-integrated exogenous polynucleotides are introduced using Sendai virus, AAV, episomal, or plasmid. Methods of generating any of the genetically modified cells described herein are contemplated to be performed using but not limited to, any of the gene editing methods described herein. Additional description related to how to generate genetically modified cells in vitro can be found, for example, in PCT/IB2023/055621 and WO 2022/113056, the contents of which are incorporated herein by reference in their entirety.

In some embodiments, in vitro method for generating an engineered cell can comprise delivering to a cell: (i) an RNP complex comprising an RNA-guided endonuclease and a gRNA targeting a target site in a FAS gene locus or an RNA-guided endonuclease and a gRNA targeting a target site in the FAS gene locus, wherein the FAS gene locus is cleaved at the target site, thereby disrupting the FAS gene; (ii) a nucleic acid encoding IL15; and (iii) a nucleic acid encoding a CAR. In some embodiments, the CAR is an anti-GPR87 CAR. In some embodiments, the nucleic acid encoding the IL15 is provided in a vector. In some embodiments, the nucleic acid encoding the CAR is provided in a vector. The nucleic acids encoding the IL15 and CAR can be provided in a same vector or two separate vectors.

The delivery of (i), (ii) and (iii) can be performed sequentially or simultaneously. In some embodiments, the RNP complex comprising the RNA-guided endonuclease and the gRNA targeting the target site in the FAS gene locus or the RNA-guided endonuclease and the gRNA targeting the target site in the FAS gene locus are delivered to the cell following the delivery of the nucleic acid encoding IL15 and/or the nucleic acid encoding the CAR. In some embodiments, the nucleic acid encoding IL15 and/or the nucleic acid encoding the CAR are delivered to the cell following the delivery of the RNP complex comprising the RNA-guided endonuclease and the gRNA targeting the target site in the FAS gene locus or the RNA-guided endonuclease and the gRNA targeting the target site in the FAS gene locus.

In some embodiments, the method further comprises delivering to the cell: an RNP complex comprising an RNA-guided endonuclease and a gRNA targeting a target site in a CISH gene locus or an RNA-guided endonuclease and a gRNA targeting a target site in the CISH gene locus, wherein the CISH gene locus is cleaved at the target site, thereby disrupting the CISH gene;

The gRNA targeting the target site in the FAS gene locus can comprise a spacer sequence of any one of SEQ ID NOs: 55-60 and 63. In some embodiments, the gRNA targeting the target site in the FAS gene locus comprises a sequence differing by one, two, three, four, or five mismatches relative to any one of the sequences of SEQ ID NOs: 55-60 and 63. In some embodiments, the gRNA targeting the target site in the FAS gene locus consists of a sequence selected from the sequences of SEQ ID NOs: 55-60 and 63. The gRNA targeting the target site in the FAS gene locus can comprise or consist of a spacer sequence of SEQ ID NO: 55 or SEQ ID NO: 56.

The gRNA targeting the target site in the CISH gene locus can comprise a spacer sequence of any one of SEQ ID NOs: 64-75. In some embodiments, the gRNA targeting the target site in the CISH gene locus comprises a sequence differing by one, two, three, four, or five mismatches relative to any one of the sequences of SEQ ID NOs: 64-75. In some embodiments, the gRNA targeting the target site in the CISH gene locus consists of a sequence selected from the sequences of SEQ ID NOs: 64-75. The gRNA targeting the target site in the CISH gene locus can comprise or consist of a spacer sequence of SEQ ID NO: 65.

The nucleotide sequence encoding the anti-GPR87 CAR can comprise the sequence of SEQ ID NO: 47, or a polynucleotide sequence having at least 85%, 90%, 95%, or 99% sequence identity to SEQ ID NO: 47. In some embodiments, the nucleotide sequence encoding the anti-GPR87 CAR consists of the sequence of SEQ ID NO: 47. The nucleotide sequence encoding the anti-GPC3 CAR can comprise the sequence of SEQ ID NO: 45, or a polynucleotide sequence having at least 85%, 90%, 95%, or 99% sequence identity to SEQ ID NO: 45. In some embodiments, the nucleotide sequence encoding the anti-GPC3 CAR consists of the sequence of SEQ ID NO: 45.

In some embodiments, the engineered cells of the disclosure (e.g., cells generated by the gene editing methods disclosed herein) have disrupted expression of the gene products (e.g., a protein encoded by) of the FAS gene locus, the CISH gene locus, or both. In some embodiments, the disrupted expression comprises reduced or eliminated expression as compared to, e.g., unedited cells. In some embodiments, the disrupted expression comprises about, at least, or at least about 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% or more, reduction in expression as compared to, e.g., an unedited cell. In some embodiments, a population of edited cells exhibits disrupted expression of the FAS mRNA or protein. The disrupted expression of the FAS gene can be about, at least, or at least about 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% or more, reduction in expression as compared to, e.g., a population of unedited cells.

In some embodiments, the engineered cells of the disclosure (e.g., cells generated by the gene editing methods disclosed herein) have increased expression of the mRNA and/or protein encoded by any of the donor polynucleotides disclosed herein. In some embodiments, the engineered cells exhibit increased expression of IL-15 and a CAR (e.g., anti-GPR87 CAR). In some embodiments, the increased expression comprises increased expression as compared to, e.g., unedited cells. In some embodiments, the increased expression comprises about 0.1%, 0.5%, 1%, 2%, 3%, 4%, 5%, 6%, 7%, 8%, 9%, 10%, 11%, 12%, 13%, 14%, 1%, 6%, 17%, %18%, %19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% or more increased expression as compared to, e.g., an unedited cell. In some embodiments, the engineered cells of the disclosure (e.g., cells generated by the gene editing methods disclosed herein) have increased expression of the mRNA and/or protein encoded by any of the donor polynucleotides enclosed herein. In some embodiments, a population of cell comprising the engineered cells of the disclosure exhibit increased expression of IL-15 and a CAR (e.g., anti-GPR87 CAR). In some embodiments, the increased expression comprises increased expression as compared to, e.g., a population of unedited cells. In some embodiments, the increased expression comprises about 0.1%, 0.5%, 1%, 2%, 3%, 4%, 5%, 6%, 7%, 8%, 9%, 10%, 11%, 12%, 13%, 14%, 15%, 16%, 7%, 18%, %19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% or more increased expression as compared to, e.g., a population of cells comprising unedited cells.

### Genome Editing Methods

Genome editing generally refers to the process of modifying the nucleotide sequence of a genome, preferably in a precise or pre-determined manner. In some embodiments, genome editing methods as described herein, e.g., the CRISPR-endonuclease system, are used to genetically modify a cell as described herein, e.g., to create a gene-edited iPSC.

Examples of methods of genome editing described herein include methods of using site-directed nucleases to cut deoxyribonucleic acid (DNA) at precise target locations in the genome, thereby creating single-strand or double-strand DNA breaks at particular locations within the genome. Such breaks can be and regularly are repaired by natural, endogenous cellular processes, such as homology-directed repair (HDR) and non-homologous end joining (NHEJ), as described in Cox et al., “Therapeutic genome editing: prospects and challenges,”, Nature Medicine, 2015, 21(2), 121-31. These two main DNA repair processes consist of a family of alternative pathways. NHEJ directly joins the DNA ends resulting from a double-strand break, sometimes with the loss or addition of nucleotide sequence, which may disrupt or enhance gene expression. HDR utilizes a homologous sequence, or donor sequence, as a template for inserting a defined DNA sequence at the break point. The homologous sequence can be in the endogenous genome, such as a sister chromatid. Alternatively, the donor sequence can be an exogenous polynucleotide, such as a plasmid, a single-strand oligonucleotide, a double-stranded oligonucleotide, a duplex oligonucleotide or a virus, that has regions (e.g., left and right homology arms) of high homology with the nuclease-cleaved locus, but which can also contain additional sequence or sequence changes including deletions that can be incorporated into the cleaved target locus. A third repair mechanism can be microhomology-mediated end joining (MMEJ), also referred to as “Alternative NHEJ,” in which the genetic outcome is similar to NHEJ in that small deletions and insertions can occur at the cleavage site. MMEJ can make use of homologous sequences of a few base pairs flanking the DNA break site to drive a more favored DNA end joining repair outcome, and recent reports have further elucidated the molecular mechanism of this process; see, e.g., Cho and Greenberg, Nature, 2015, 518, 174-76; Kent et al., Nature Structural and Molecular Biology, 2015, 22(3):230-7; Mateos-Gomez et al., Nature, 2015, 518, 254-57; Ceccaldi et al., Nature, 2015, 528, 258-62. In some instances, it may be possible to predict likely repair outcomes based on analysis of potential microhomologies at the site of the DNA break.

Each of these genome editing mechanisms can be used to create desired genetic modifications. A step in the genome editing process can be to create one or two DNA breaks, the latter as double-strand breaks or as two single-stranded breaks, in the target locus as near the site of intended mutation. This can be achieved via the use of endonucleases, as described herein.

### CRISPR Endonuclease System

The CRISPR-endonuclease system is a naturally occurring defense mechanism in prokaryotes that has been repurposed as an RNA-guided DNA-targeting platform used for gene editing. CRISPR systems include Types I, II, III, IV, V, and VI systems. In some aspects, the CRISPR system is a Type II CRISPR/Cas9 system. In other aspects, the CRISPR system is a Type V CRISPR/Cprf system. CRISPR systems rely on a DNA endonuclease, e.g., Cas9, and two noncoding RNAs, crisprRNA (crRNA) and trans-activating RNA (tracrRNA), to target the cleavage of DNA.

The crRNA drives sequence recognition and specificity of the CRISPR-endonuclease complex through Watson-Crick base pairing, typically with a ˜20 nucleotide (nt) sequence in the target DNA. Changing the sequence of the 5′ 20 nt in the crRNA (e.g., the spacer sequence) allows targeting of the CRISPR-endonuclease complex to specific loci. The CRISPR-endonuclease complex only binds DNA sequences that contain a sequence match to the first 20 nt of the single-guide RNA (sgRNA) if the target sequence is followed by a specific short DNA motif (with the sequence NGG for some Cas proteins) referred to as a protospacer adjacent motif (PAM). TracrRNA hybridizes with the 3′ end of crRNA to form an RNA-duplex structure that is bound by the endonuclease to form the catalytically active CRISPR-endonuclease complex, which can then cleave the target DNA. Once the CRISPR-endonuclease complex is bound to DNA at a target site, two independent nuclease domains within the endonuclease each cleave one of the DNA strands three bases upstream of the PAM site, leaving a double-strand break (DSB) where both strands of the DNA terminate in a base pair (a blunt end).

The endonuclease can be a Cas9 (CRISPR associated protein 9). The Cas9 endonuclease can be, for example, Cas9 from Streptococcus pyogenes, S. aureus Cas9, N. meningitidis Cas9, S. thermophilus CRISPR 1 Cas9, S. thermophilus CRISPR 3 Cas9, or T. denticola Cas9. In some embodiments, the CRISPR endonuclease is Cpf1, e.g., L. bacterium ND2006 Cpf1 or Acidaminococcus sp. BV3L6 Cpf1. In some embodiments, the endonuclease is Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease. In some embodiments, wild-type variants may be used. In some embodiments, modified versions (e.g., a homolog thereof, a recombination of the naturally occurring molecule thereof, codon-optimized thereof, or modified versions thereof) of the preceding endonucleases may be used. The CRISPR nuclease can be linked to at least one nuclear localization signal (NLS). The at least one NLS can be located at or within 50 amino acids of the amino-terminus of the CRISPR nuclease and/or at least one NLS can be located at or within 50 amino acids of the carboxy-terminus of the CRISPR nuclease.

Exemplary CRISPR/Cas polypeptides include the Cas9 polypeptides as published in Fonfara et al., “Phylogeny of Cas9 determines functional exchangeability of dual-RNA and Cas9 among orthologous type II CRISPR-Cas systems,” Nucleic Acids Research, 2014, 42: 2577-2590. The CRISPR/Cas gene naming system has undergone extensive rewriting since the Cas genes were discovered. Fonfara et al. also provides PAM sequences for the Cas9 polypeptides from various species.

### Zinc Finger Nucleases

Zinc finger nucleases (ZFNs) are modular proteins comprised of an engineered zinc finger DNA binding domain linked to the catalytic domain of the type II endonuclease FokI. Because FokI functions only as a dimer, a pair of ZFNs must be engineered to bind to cognate target “half-site” sequences on opposite DNA strands and with precise spacing between them to enable the catalytically active FokI dimer to form. Upon dimerization of the FokI domain, which itself has no sequence specificity per se, a DNA double-strand break is generated between the ZFN half-sites as the initiating step in genome editing.

The DNA binding domain of each ZFN is typically comprised of 3-6 zinc fingers of the abundant Cys2-His2 architecture, with each finger primarily recognizing a triplet of nucleotides on one strand of the target DNA sequence, although cross-strand interaction with a fourth nucleotide also can be important. Alteration of the amino acids of a finger in positions that make key contacts with the DNA alters the sequence specificity of a given finger. Thus, a four-finger zinc finger protein will selectively recognize a 12 bp target sequence, where the target sequence is a composite of the triplet preferences contributed by each finger, although triplet preference can be influenced to varying degrees by neighboring fingers. An important aspect of ZFNs is that they can be readily re-targeted to almost any genomic address simply by modifying individual fingers. In most applications of ZFNs, proteins of 4-6 fingers are used, recognizing 12-18 bp respectively. Hence, a pair of ZFNs will typically recognize a combined target sequence of 24-36 bp, not including the typical 5-7 bp spacer between half-sites. The binding sites can be separated further with larger spacers, including 15-17 bp. A target sequence of this length is likely to be unique in the human genome, assuming repetitive sequences or gene homologs are excluded during the design process. Nevertheless, the ZFN protein-DNA interactions are not absolute in their specificity so off-target binding and cleavage events do occur, either as a heterodimer between the two ZFNs, or as a homodimer of one or the other of the ZFNs. The latter possibility has been effectively eliminated by engineering the dimerization interface of the FokI domain to create “plus” and “minus” variants, also known as obligate heterodimer variants, which can only dimerize with each other, and not with themselves. Forcing the obligate heterodimer prevents formation of the homodimer. This has greatly enhanced specificity of ZFNs, as well as any other nuclease that adopts these FokI variants.

A variety of ZFN-based systems have been described in the art, modifications thereof are regularly reported, and numerous references describe rules and parameters that are used to guide the design of ZFNs; see, e.g., Segal et al., Proc Natl Acad Sci, 1999 96(6):2758-63; Dreier B et al., J Mol Biol., 2000, 303(4):489-502; Liu Q et al., J Biol Chem., 2002, 277(6):3850-6; Dreier et al., J Biol Chem., 2005, 280(42):35588-97; and Dreier et al., J Biol Chem. 2001, 276(31):29466-78.

### Transcription Activator-Like Effector Nucleases (TALENs)

TALENs represent another format of modular nucleases whereby, as with ZFNs, an engineered DNA binding domain is linked to the FokI nuclease domain, and a pair of TALENs operate in tandem to achieve targeted DNA cleavage. The major difference from ZFNs is the nature of the DNA binding domain and the associated target DNA sequence recognition properties. The TALEN DNA binding domain derives from TALE proteins, which were originally described in the plant bacterial pathogen Xanthomonas sp. TALEs are comprised of tandem arrays of 33-35 amino acid repeats, with each repeat recognizing a single base pair in the target DNA sequence that is typically up to 20 bp in length, giving a total target sequence length of up to 40 bp. Nucleotide specificity of each repeat is determined by the repeat variable diresidue (RVD), which includes just two amino acids at positions 12 and 13. The bases guanine, adenine, cytosine and thymine are predominantly recognized by the four RVDs: Asn-Asn, Asn-Ile, His-Asp and Asn-Gly, respectively. This constitutes a much simpler recognition code than for zinc fingers, and thus represents an advantage over the latter for nuclease design. Nevertheless, as with ZFNs, the protein-DNA interactions of TALENs are not absolute in their specificity, and TALENs have also benefitted from the use of obligate heterodimer variants of the FokI domain to reduce off-target activity.

Additional variants of the FokI domain have been created that are deactivated in their catalytic function. If one half of either a TALEN or a ZFN pair contains an inactive FokI domain, then only single-strand DNA cleavage (nicking) will occur at the target site, rather than a DSB. The outcome is comparable to the use of CRISPR/Cas9 or CRISPR/Cpf1 “nickase” mutants in which one of the Cas9 cleavage domains has been deactivated. DNA nicks can be used to drive genome editing by HDR, but at lower efficiency than with a DSB. The main benefit is that off-target nicks are quickly and accurately repaired, unlike the DSB, which is prone to NHEJ-mediated mis-repair.

A variety of TALEN-based systems have been described in the art, and modifications thereof are regularly reported; see, e.g., Boch, Science, 2009 326(5959):1509-12; Mak et al., Science, 2012, 335(6069):716-9; and Moscou et al., Science, 2009, 326(5959):1501. The use of TALENs based on the “Golden Gate” platform, or cloning scheme, has been described by multiple groups; see, e.g., Cermak et al., Nucleic Acids Res., 2011, 39(12):e82; Li et al., Nucleic Acids Res., 2011, 39(14):6315-25; Weber et al., PLoS One., 2011, 6(2):e16765; Wang et al., J Genet Genomics, 2014, 41(6):339-47.; and Cermak T et al., Methods Mol Biol., 2015 1239:133-59.

### Homing Endonucleases

Homing endonucleases (HEs) are sequence-specific endonucleases that have long recognition sequences (14-44 base pairs) and cleave DNA with high specificity, often at sites unique in the genome. There are at least six known families of HEs as classified by their structure, including GIY-YIG, His-Cis box, H—N—H, PD-(D/E)xK, and Vsr-like that are derived from a broad range of hosts, including eukaryotes, protists, bacteria, archaea, cyanobacteria and phage. As with ZFNs and TALENs, HEs can be used to create a DSB at a target locus as the initial step in genome editing. In addition, some natural and engineered HEs cut only a single strand of DNA, thereby functioning as site-specific nickases. The large target sequence of HEs and the specificity that they offer have made them attractive candidates to create site-specific DSBs.

A variety of HE-based systems have been described in the art, and modifications thereof are regularly reported; see, e.g., the reviews by Steentoft et al., Glycobiology, 2014, 24(8):663-80; Belfort and Bonocora, Methods Mol Biol., 2014, 1123:1-26; and Hafez and Hausner, Genome, 2012, 55(8):553-69.

### MegaTAL/Tev-mTALEN/MegaTev

As further examples of hybrid nucleases, the MegaTAL platform and Tev-mTALEN platform use a fusion of TALE DNA binding domains and catalytically active HEs, taking advantage of both the tunable DNA binding and specificity of the TALE, as well as the cleavage sequence specificity of the HE; see, e.g., Boissel et al., Nucleic Acids Res., 2014, 42: 2591-2601; Kleinstiver et al., G3, 2014, 4:1155-65; and Boissel and Scharenberg, Methods Mol. Biol., 2015, 1239: 171-96.

In a further variation, the MegaTev architecture is the fusion of a meganuclease (Mega) with the nuclease domain derived from the GIY-YIG homing endonuclease I-TevI (Tev). The two active sites are positioned ˜30 bp apart on a DNA substrate and generate two DSBs with non-compatible cohesive ends; see, e.g., Wolfs et al., Nucleic Acids Res., 2014, 42, 8816-29. It is anticipated that other combinations of existing nuclease-based approaches will evolve and be useful in achieving the targeted genome modifications described herein.

dCas9-FokI or dCpf1-FokI and Other Nucleases

Combining the structural and functional properties of the nuclease platforms described above offers a further approach to genome editing that can potentially overcome some of the inherent deficiencies. As an example, the CRISPR genome editing system typically uses a single Cas9 endonuclease to create a DSB. The specificity of targeting is driven by a 20 or 24 nucleotide sequence in the guide RNA that undergoes Watson-Crick base-pairing with the target DNA (plus an additional 2 bases in the adjacent NAG or NGG PAM sequence in the case of Cas9 from S. pyogenes). Such a sequence is long enough to be unique in the human genome, however, the specificity of the RNA/DNA interaction is not absolute, with significant promiscuity sometimes tolerated, particularly in the 5′ half of the target sequence, effectively reducing the number of bases that drive specificity. One solution to this has been to completely deactivate the Cas9 or Cpf1 catalytic function—retaining only the RNA-guided DNA binding function—and instead fusing a FokI domain to the deactivated Cas9; see, e.g., Tsai et al., Nature Biotech, 2014, 32: 569-76; and Guilinger et al., Nature Biotech., 2014, 32: 577-82. Because FokI must dimerize to become catalytically active, two guide RNAs are required to tether two FokI fusions in close proximity to form the dimer and cleave DNA. This essentially doubles the number of bases in the combined target sites, thereby increasing the stringency of targeting by CRISPR-based systems.

As further example, fusion of the TALE DNA binding domain to a catalytically active HE, such as I-TevI, takes advantage of both the tunable DNA binding and specificity of the TALE, as well as the cleavage sequence specificity of I-TevI, with the expectation that off-target cleavage can be further reduced.

### Base Editing

In some embodiments, a gene is edited in a cell using base editing. Base Editing is a technique enabling the conversion of one nucleotide into another without double-stranded breaks in the DNA. Base editing allows for conversion of a C to T, G to A, or vice versa. An example editor for cytosine includes rAPOBEC1 which is fused to a catalytically inactive form of Cas9. The Cas9 helps to bind a site of interest and the rAPOBEC1 cytidine deaminase induces the point mutation. Conversion of adenine requires a mutant transfer RNA adenosine deaminase (TadA), a Cas9 nickase, and a sgRNA, as described herein. The construct is able to introduce the site-specific mutation without introducing a strand break. In some embodiments, Base Editing is used to introduce one or more mutations in a cell described herein.

### RNA-Guided Endonucleases

The RNA-guided endonuclease systems as used herein can comprise an amino acid sequence having at least 10%, at least 15%, at least 20%, at least 30%, at least 40%, at least 50%, at least 60%, at least 70%, at least 75%, at least 80%, at least 85%, at least 90%, at least 95%, at least 99%, or 100% amino acid sequence identity to a wild-type exemplary endonuclease, e.g., Cas9 from S. pyogenes, US2014/0068797 SEQ ID NO: 8 or Sapranauskas et al., Nucleic Acids Res, 39(21): 9275-9282 (2011). The endonuclease can comprise at least 70, 75, 80, 85, 90, 95, 97, 99, or 100% identity to a wild-type endonuclease (e.g., Cas9 from S. pyogenes, supra) over 10 contiguous amino acids. The endonuclease can comprise at most: 70, 75, 80, 85, 90, 95, 97, 99, or 100% identity to a wild-type endonuclease (e.g., Cas9 from S. pyogenes, supra) over 10 contiguous amino acids. The endonuclease can comprise at least: 70, 75, 80, 85, 90, 95, 97, 99, or 100% identity to a wild-type endonuclease (e.g., Cas9 from S. pyogenes, supra) over 10 contiguous amino acids in a HNH nuclease domain of the endonuclease. The endonuclease can comprise at most: 70, 75, 80, 85, 90, 95, 97, 99, or 100% identity to a wild-type endonuclease (e.g., Cas9 from S. pyogenes, supra) over 10 contiguous amino acids in a HNH nuclease domain of the endonuclease. The endonuclease can comprise at least: 70, 75, 80, 85, 90, 95, 97, 99, or 100% identity to a wild-type endonuclease (e.g., Cas9 from S. pyogenes, supra) over 10 contiguous amino acids in a RuvC nuclease domain of the endonuclease. The endonuclease can comprise at most: 70, 75, 80, 85, 90, 95, 97, 99, or 100% identity to a wild-type endonuclease (e.g., Cas9 from S. pyogenes, supra) over 10 contiguous amino acids in a RuvC nuclease domain of the endonuclease.

The endonuclease can comprise a modified form of a wild-type exemplary endonuclease. The modified form of the wild-type exemplary endonuclease can comprise a mutation that reduces the nucleic acid-cleaving activity of the endonuclease. The modified form of the wild-type exemplary endonuclease can have less than 90%, less than 80%, less than 70%, less than 60%, less than 50%, less than 40%, less than 30%, less than 20%, less than 10%, less than 5%, or less than 1% of the nucleic acid-cleaving activity of the wild-type exemplary endonuclease (e.g., Cas9 from S. pyogenes, supra). The modified form of the endonuclease can have no substantial nucleic acid-cleaving activity. When an endonuclease is a modified form that has no substantial nucleic acid-cleaving activity, it is referred to herein as “enzymatically inactive.”

Mutations contemplated can include substitutions, additions, and deletions, or any combination thereof. The mutation can convert the mutated amino acid to alanine. The mutation can convert the mutated amino acid to another amino acid (e.g., glycine, serine, threonine, cysteine, valine, leucine, isoleucine, methionine, proline, phenylalanine, tyrosine, tryptophan, aspartic acid, glutamic acid, asparagine, glutamine, histidine, lysine, or arginine). The mutation can convert the mutated amino acid to a non-natural amino acid (e.g., selenomethionine). The mutation can convert the mutated amino acid to amino acid mimics (e.g., phosphomimics). The mutation can be a conservative mutation. For example, the mutation can convert the mutated amino acid to an amino acids that resemble the size, shape, charge, polarity, conformation, and/or rotamers of the mutated amino acids (e.g., cysteine/serine mutation, lysine/asparagine mutation, histidine/phenylalanine mutation). The mutation can cause a shift in reading frame and/or the creation of a premature stop codon. Mutations can cause changes to regulatory regions of genes or loci that affect expression of one or more genes.

### Guide RNAs

Provided herein include guide RNAs (gRNAs) that can direct the activities of an associated endonuclease to a specific target site within a polynucleotide. A guide RNA can comprise at least a spacer sequence that hybridizes to a target nucleic acid sequence of interest, and a CRISPR repeat sequence. In CRISPR Type II systems, the gRNA also comprises a second RNA called the tracrRNA sequence. In the CRISPR Type II guide RNA (gRNA), the CRISPR repeat sequence and tracrRNA sequence hybridize to each other to form a duplex. In CRISPR Type V systems, the gRNA comprises a crRNA that forms a duplex. In some embodiments, a gRNA can bind an endonuclease, such that the gRNA and endonuclease form a complex. The gRNA can provide target specificity to the complex by virtue of its association with the endonuclease. The genome-targeting nucleic acid thus can direct the activity of the endonuclease.

Exemplary guide RNAs include a spacer sequences that comprises 15-200 nucleotides wherein the gRNA targets a genome location based on the GRCh38 human genome assembly. As is understood by the person of ordinary skill in the art, each gRNA can be designed to include a spacer sequence complementary to its genomic target site or region, i.e., the “target sequence.” The “target sequence” is in a target gene that is adjacent to a PAM sequence and is the sequence to be modified by Cas9. The “target sequence” is on the so-called PAM-strand in a “target nucleic acid,” which is a double-stranded molecule containing the PAM-strand and a complementary non-PAM strand. One of skill in the art recognizes that the gRNA spacer sequence hybridizes to the complementary sequence located in the non-PAM strand of the target nucleic acid of interest. Thus, the gRNA spacer sequence is the RNA equivalent of the target sequence. See Jinek et al., Science, 2012, 337, 816-821 and Deltcheva et al., Nature, 2011, 471, 602-607. For example, as described herein, a gRNA can comprise an RNA spacer sequence corresponding to the target DNA sequence (i.e., the RNA space sequence is the target DNA sequence in which T is substituted for U).

The gRNA can be a double-molecule guide RNA. The gRNA can be a single-molecule guide RNA. A double-molecule guide RNA can comprise two strands of RNA. The first strand comprises in the 5′ to 3′ direction, an optional spacer extension sequence, a spacer sequence and a minimum CRISPR repeat sequence. The second strand can comprise a minimum tracrRNA sequence (complementary to the minimum CRISPR repeat sequence), a 3′ tracrRNA sequence and an optional tracrRNA extension sequence.

A single-molecule guide RNA (sgRNA) can comprise, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension can comprise elements that contribute additional functionality (e.g., stability) to the guide RNA. The single-molecule guide linker can link the minimum CRISPR repeat and the minimum tracrRNA sequence to form a hairpin structure. The optional tracrRNA extension can comprise one or more hairpins.

In some embodiments, an sgRNA comprises a 20 nucleotide spacer sequence at the 5′ end of the sgRNA sequence. In some embodiments, an sgRNA comprises a less than a 20 nucleotide spacer sequence at the 5′ end of the sgRNA sequence. In some embodiments, an sgRNA comprises a more than 20 nucleotide spacer sequence at the 5′ end of the sgRNA sequence. In some embodiments, an sgRNA comprises a variable length spacer sequence with 17-30 nucleotides at the 5′ end of the sgRNA sequence. In some embodiments, an sgRNA comprises a spacer extension sequence with a length of more than 1, 5, 10, 15, 20, 25, 30, 35, 40, 45, 50, 60, 70, 80, 90, 100, 120, 140, 160, 180, or 200 nucleotides. In some embodiments, an sgRNA comprises a spacer extension sequence with a length of less than 3, 5, 10, 15, 20, 25, 30, 35, 40, 45, 50, 60, 70, 80, 90, or 100 nucleotides.

An sgRNA can comprise a spacer extension sequence that comprises another moiety (e.g., a stability control sequence, an endoribonuclease binding sequence, or a ribozyme). The moiety can decrease or increase the stability of a nucleic acid targeting nucleic acid. The moiety can be a transcriptional terminator segment (i.e., a transcription termination sequence). The moiety can function in a eukaryotic cell. The moiety can function in a prokaryotic cell. The moiety can function in both eukaryotic and prokaryotic cells. Non-limiting examples of suitable moieties include: a 5′ cap (e.g., a 7-methylguanylate cap (m7 G)), a riboswitch sequence (e.g., to allow for regulated stability and/or regulated accessibility by proteins and protein complexes), a sequence that forms a dsRNA duplex (i.e., a hairpin), a sequence that targets the RNA to a subcellular location (e.g., nucleus, mitochondria, chloroplasts, and the like), a modification or sequence that provides for tracking (e.g., direct conjugation to a fluorescent molecule, conjugation to a moiety that facilitates fluorescent detection, a sequence that allows for fluorescent detection, etc.), and/or a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like).

A sgRNA can comprise a spacer sequence that hybridizes to a sequence in a target polynucleotide. The spacer of a gRNA can interact with a target polynucleotide in a sequence-specific manner via hybridization (i.e., base pairing). The nucleotide sequence of the spacer can vary depending on the sequence of the target nucleic acid of interest. In a CRISPR-endonuclease system, a spacer sequence can be designed to hybridize to a target polynucleotide that is located 5′ of a PAM of the endonuclease used in the system. The spacer may perfectly match the target sequence or may have mismatches. Each endonuclease, e.g., Cas9 nuclease, has a particular PAM sequence that it recognizes in a target DNA. For example, S. pyogenes Cas9 recognizes a PAM that comprises the sequence 5′-NRG-3′, where R comprises either A or G, where N is any nucleotide and N is immediately 3′ of the target nucleic acid sequence targeted by the spacer sequence.

A target polynucleotide sequence can comprise 20 nucleotides. The target polynucleotide can comprise less than 20 nucleotides. The target polynucleotide can comprise more than 20 nucleotides. The target polynucleotide can comprise at least: 5, 10, 15, 16, 17, 18, 19, 20, 21, 22, 23, 24, 25, 30 or more nucleotides. The target polynucleotide can comprise at most: 5, 10, 15, 16, 17, 18, 19, 20, 21, 22, 23, 24, 25, 30 or more nucleotides. The target polynucleotide sequence can comprise 20 bases immediately 5′ of the first nucleotide of the PAM.

A spacer sequence that hybridizes to a target polynucleotide can have a length of at least about 6 nucleotides (nt). The spacer sequence can be at least about 6 nt, at least about 10 nt, at least about 15 nt, at least about 18 nt, at least about 19 nt, at least about 20 nt, at least about 25 nt, at least about 30 nt, at least about 35 nt or at least about 40 nt, from about 6 nt to about 80 nt, from about 6 nt to about 50 nt, from about 6 nt to about 45 nt, from about 6 nt to about 40 nt, from about 6 nt to about 35 nt, from about 6 nt to about 30 nt, from about 6 nt to about 25 nt, from about 6 nt to about 20 nt, from about 6 nt to about 19 nt, from about 10 nt to about 50 nt, from about 10 nt to about 45 nt, from about 10 nt to about 40 nt, from about 10 nt to about 35 nt, from about 10 nt to about 30 nt, from about 10 nt to about 25 nt, from about 10 nt to about 20 nt, from about 10 nt to about 19 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. In some examples, the spacer sequence can comprise 20 nucleotides. In some examples, the spacer can comprise 19 nucleotides. In some examples, the spacer can comprise 18 nucleotides. In some examples, the spacer can comprise 22 nucleotides.

In some examples, the percent complementarity between the spacer sequence and the target nucleic acid is at least about 30%, at least about 40%, at least about 50%, at least about 60%, at least about 65%, at least about 70%, at least about 75%, at least about 80%, at least about 85%, at least about 90%, at least about 95%, at least about 97%, at least about 98%, at least about 99%, or 100%. In some examples, the percent complementarity between the spacer sequence and the target nucleic acid is at most about 30%, at most about 40%, at most about 50%, at most about 60%, at most about 65%, at most about 70%, at most about 75%, at most about 80%, at most about 85%, at most about 90%, at most about 95%, at most about 97%, at most about 98%, at most about 99%, or 100%. In some examples, the percent complementarity between the spacer sequence and the target nucleic acid is 100% over the six contiguous 5′-most nucleotides of the target sequence of the complementary strand of the target nucleic acid. The percent complementarity between the spacer sequence and the target nucleic acid can be at least 60% over about 20 contiguous nucleotides. The length of the spacer sequence and the target nucleic acid can differ by 1 to 6 nucleotides, which may be thought of as a bulge or bulges.

A tracrRNA sequence can comprise nucleotides that hybridize to a minimum CRISPR repeat sequence in a cell. A minimum tracrRNA sequence and a minimum CRISPR repeat sequence may form a duplex, i.e., a base-paired double-stranded structure. Together, the minimum tracrRNA sequence and the minimum CRISPR repeat can bind to an RNA-guided endonuclease. At least a part of the minimum tracrRNA sequence can hybridize to the minimum CRISPR repeat sequence. The minimum tracrRNA sequence can be at least about 30%, about 40%, about 50%, about 60%, about 65%, about 70%, about 75%, about 80%, about 85%, about 90%, about 95%, or 100% complementary to the minimum CRISPR repeat sequence.

The minimum tracrRNA sequence can have a length from about 7 nucleotides to about 100 nucleotides. For example, the minimum tracrRNA sequence can be from about 7 nucleotides (nt) to about 50 nt, from about 7 nt to about 40 nt, from about 7 nt to about 30 nt, from about 7 nt to about 25 nt, from about 7 nt to about 20 nt, from about 7 nt to about 15 nt, from about 8 nt to about 40 nt, from about 8 nt to about 30 nt, from about 8 nt to about 25 nt, from about 8 nt to about 20 nt, from about 8 nt to about 15 nt, from about 15 nt to about 100 nt, from about 15 nt to about 80 nt, from about 15 nt to about 50 nt, from about 15 nt to about 40 nt, from about 15 nt to about 30 nt or from about 15 nt to about 25 nt long. The minimum tracrRNA sequence can be approximately 9 nucleotides in length. The minimum tracrRNA sequence can be approximately 12 nucleotides. The minimum tracrRNA can consist of tracrRNA nt 23-48 described in Jinek et al., supra.

The minimum tracrRNA sequence can be at least about 60% identical to a reference minimum tracrRNA (e.g., wild type, tracrRNA from S. pyogenes) sequence over a stretch of at least 6, 7, or 8 contiguous nucleotides. For example, the minimum tracrRNA sequence can be at least about 65% identical, about 70% identical, about 75% identical, about 80% identical, about 85% identical, about 90% identical, about 95% identical, about 98% identical, about 99% identical or 100% identical to a reference minimum tracrRNA sequence over a stretch of at least 6, 7, or 8 contiguous nucleotides.

The duplex between the minimum CRISPR RNA and the minimum tracrRNA can comprise a double helix. The duplex between the minimum CRISPR RNA and the minimum tracrRNA can comprise at least about 1, 2, 3, 4, 5, 6, 7, 8, 9, or 10 or more nucleotides. The duplex between the minimum CRISPR RNA and the minimum tracrRNA can comprise at most about 1, 2, 3, 4, 5, 6, 7, 8, 9, or 10 or more nucleotides. The duplex can comprise a mismatch (i.e., the two strands of the duplex are not 100% complementary). The duplex can comprise at least about 1, 2, 3, 4, or 5 or mismatches. The duplex can comprise at most about 1, 2, 3, 4, or 5 or mismatches. The duplex can comprise no more than 2 mismatches.

In some embodiments, a tracrRNA may be a 3′ tracrRNA. In some embodiments, a 3′ tracrRNA sequence can comprise a sequence with at least about 30%, about 40%, about 50%, about 60%, about 65%, about 70%, about 75%, about 80%, about 85%, about 90%, about 95%, or 100% sequence identity to a reference tracrRNA sequence (e.g., a tracrRNA from S. pyogenes).

In some embodiments, a gRNA comprises a tracrRNA extension sequence. A tracrRNA extension sequence can have a length from about 1 nucleotide to about 400 nucleotides. The tracrRNA extension sequence can have a length of more than 1, 5, 10, 15, 20, 25, 30, 35, 40, 45, 50, 60, 70, 80, 90, 100, 120, 140, 160, 180, or 200 nucleotides. The tracrRNA extension sequence can have a length from about 20 to about 5000 or more nucleotides. The tracrRNA extension sequence can have a length of less than 5, 10, 15, 20, 25, 30, 35, 40, 45, 50, 60, 70, 80, 90, or 100 nucleotides. The tracrRNA extension sequence can comprise less than 10 nucleotides in length. The tracrRNA extension sequence can be 10-30 nucleotides in length. The tracrRNA extension sequence can be 30-70 nucleotides in length. The tracrRNA extension sequence can comprise a functional moiety (e.g., a stability control sequence, ribozyme, endoribonuclease binding sequence). The functional moiety can comprise a transcriptional terminator segment (i.e., a transcription termination sequence). The functional moiety can have a total length from about 10 nt to about 100 nt, from about 10 nt to about 20 nt, from about 20 nt to about 30 nt, from about 30 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt, from about 15 nt to about 80 nt, from about 15 nt to about 50 nt, from about 15 nt to about 40 nt, from about 15 nt to about 30 nt, or from about 15 nt to about 25 nt.

In some embodiments, an sgRNA comprise a linker sequence with a length from about 3 nt to about 100 nt. In Jinek et al., supra, for example, a simple 4 nucleotide “tetraloop” (-GAAA-) was used (Jinek et al., Science, 2012, 337(6096):816-821). An illustrative linker has a length from about 3 nt to about 90 nt, from about 3 nt to about 80 nt, from about 3 nt to about 70 nt, from about 3 nt to about 60 nt, from about 3 nt to about 50 nt, from about 3 nt to about 40 nt, from about 3 nt to about 30 nt, from about 3 nt to about 20 nt, from about 3 nt to about 10 nt. For example, the linker can have a length from about 3 nt to about 5 nt, from about 5 nt to about 10 nt, from about 10 nt to about 15 nt, from about 15 nt to about 20 nt, from about 20 nt to about 25 nt, from about 25 nt to about 30 nt, from about 30 nt to about 35 nt, from about 35 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. The linker of a single-molecule guide nucleic acid can be between 4 and 40 nucleotides. The linker can be at least about 100, 500, 1000, 1500, 2000, 2500, 3000, 3500, 4000, 4500, 5000, 5500, 6000, 6500, or 7000 or more nucleotides. The linker can be at most about 100, 500, 1000, 1500, 2000, 2500, 3000, 3500, 4000, 4500, 5000, 5500, 6000, 6500, or 7000 or more nucleotides.

Linkers can comprise any of a variety of sequences, although in some examples the linker does not comprise sequences that have extensive regions of homology with other portions of the guide RNA, which might cause intramolecular binding that could interfere with other functional regions of the guide. In Jinek et al., supra, a simple 4 nucleotide sequence -GAAA- was used (Jinek et al., Science, 2012, 337(6096):816-821), but numerous other sequences, including longer sequences can likewise be used. The linker sequence can comprise a functional moiety. For example, the linker sequence can comprise one or more features, including an aptamer, a ribozyme, a protein-interacting hairpin, a protein binding site, a CRISPR array, an intron, or an exon. The linker sequence can comprise at least about 1, 2, 3, 4, or 5 or more functional moieties. In some examples, the linker sequence can comprise at most about 1, 2, 3, 4, or 5 or more functional moieties.

In some embodiments, a sgRNA does not comprise a uracil, e.g., at the 3′end of the sgRNA sequence. In some embodiments, a sgRNA does comprise one or more uracils, e.g., at the 3′end of the sgRNA sequence. In some embodiments, an sgRNA comprises 1, 2, 3, 4, 5, 6, 7, 8, 9, or 10 uracils (U) at the 3′ end of the sgRNA sequence.

A sgRNA may be chemically modified. In some embodiments, a chemically modified gRNA is a gRNA that comprises at least one nucleotide with a chemical modification, e.g., a 2′-O-methyl sugar modification. In some embodiments, a chemically modified gRNA comprises a modified nucleic acid backbone. In some embodiments, a chemically modified gRNA comprises a 2′-O-methyl-phosphorothioate residue. In some embodiments, chemical modifications enhance stability, reduce the likelihood or degree of innate immune response, and/or enhance other attributes, as described in the art. In some embodiments, a modified gRNA may comprise a modified backbone, for example, phosphorothioates, phosphotriesters, morpholinos, methyl phosphonates, short chain alkyl or cycloalkyl intersugar linkages or short chain heteroatomic or heterocyclic intersugar linkages.

Morpholino-based compounds are described in Braasch and David Corey, Biochemistry, 2002, 41(14): 4503-4510; Genesis, 2001, Volume 30, Issue 3; Heasman, Dev. Biol., 2002, 243: 209-214; Nasevicius et al., Nat. Genet., 2000, 26:216-220; Lacerra et al., Proc. Natl. Acad. Sci., 2000, 97: 9591-9596.; and U.S. Pat. No. 5,034,506, issued Jul. 23, 1991. Cyclohexenyl nucleic acid oligonucleotide mimetics are described in Wang et al., J. Am. Chem. Soc., 2000, 122: 8595-8602.

In some embodiments, a modified gRNA may comprise one or more substituted sugar moieties, e.g., one of the following at the 2′ position: OH, SH, SCH3, F, OCN, OCH3, OCH3 O(CH2)n CH3, O(CH2)n NH2, or O(CH2)n CH3, where n is from 1 to about 10; C1 to C10 lower alkyl, alkoxyalkoxy, substituted lower alkyl, alkaryl or aralkyl; Cl; Br; CN; CF3; OCF3; O-, S-, or N-alkyl; O-, S-, or N-alkenyl; SOCH3; SO2 CH3; ONO2; NO2; N3; NH2; heterocycloalkyl; heterocycloalkaryl; aminoalkylamino; polyalkylamino; substituted silyl; an RNA cleaving group; a reporter group; an intercalator; 2′-O-(2-methoxyethyl); 2′-methoxy (2′-O—CH3); 2′-propoxy (2′-OCH2 CH2CH3); and 2′-fluoro (2′-F). Similar modifications may also be made at other positions on the gRNA, particularly the 3′ position of the sugar on the 3′ terminal nucleotide and the 5′ position of 5′ terminal nucleotide. In some examples, both a sugar and an internucleoside linkage, i.e., the backbone, of the nucleotide units can be replaced with novel groups.

Guide RNAs can also include, additionally or alternatively, nucleobase (often referred to in the art simply as “base”) modifications or substitutions. As used herein, “unmodified” or “natural” nucleobases include adenine (A), guanine (G), thymine (T), cytosine (C), and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine, and 2,6-diaminopurine. Kornberg, A., DNA Replication, W. H. Freeman & Co., San Francisco, pp 75-77, 1980; Gebeyehu et al., Nucl. Acids Res. 1997, 15:4513. A “universal” base known in the art, e.g., inosine, can also be included. 5-Me-C substitutions have been shown to increase nucleic acid duplex stability by 0.6-1.2° C. (Sanghvi, Y. S., in Crooke, S. T. and Lebleu, B., eds., Antisense Research and Applications, CRC Press, Boca Raton, 1993, pp. 276-278) and are aspects of base substitutions.

Modified nucleobases can comprise other synthetic and natural nucleobases, such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine, and 3-deazaguanine and 3-deazaadenine.

### Complexes of a Genome-Targeting Nucleic Acid and an Endonuclease

A gRNA interacts with an endonuclease (e.g., a RNA-guided nuclease such as Cas9), thereby forming a complex. The gRNA guides the endonuclease to a target polynucleotide. The endonuclease and gRNA can each be administered separately to a cell or a subject. In some embodiments, the endonuclease can be pre-complexed with one or more guide RNAs, or one or more crRNA together with a tracrRNA. The pre-complexed material can then be administered to a cell or a subject. Such pre-complexed material is known as a ribonucleoprotein particle (RNP). The endonuclease in the RNP can be, for example, a Cas9 endonuclease or a Cpf1 endonuclease. The endonuclease can be flanked at the N-terminus, the C-terminus, or both the N-terminus and C-terminus by one or more nuclear localization signals (NLSs). For example, a Cas9 endonuclease can be flanked by two NLSs, one NLS located at the N-terminus and the second NLS located at the C-terminus. The NLS can be any NLS known in the art, such as a SV40 NLS. The weight ratio of genome-targeting nucleic acid to endonuclease in the RNP can be 1:1. For example, the weight ratio of sgRNA to Cas9 endonuclease in the RNP can be 1:1.

### Cells

Disclosed herein include populations of cells comprising one or more engineered cells described herein. In some embodiments, the population or populations of lineage-restricted progenitor cells or fully differentiated somatic cells derived from one or more engineered cells are described herein.

Cells described herein can have any of the gene-edits described herein. In some embodiments, a cell (and corresponding unmodified cell) is a mammalian cell. In some embodiments, a cell (and corresponding unmodified cell) is a human cell. In some embodiments, a cell (and corresponding unmodified cell) is a stem cell. In some embodiments, a cell (and corresponding unmodified cell) is a pluripotent stem cell (PSC). In some embodiments, a cell (and corresponding unmodified cell) is an embryonic stem cell (ESC), an adult stem cell (ASC), an induced pluripotent stem cell (iPSC), or a hematopoietic stem or progenitor cell (HSPC). In some embodiments, a cell is an iPSC. In some embodiments, a cell may be a differentiated cell. In some embodiments, a cell is a somatic cell, e.g., an immune system cell or a contractile cell, e.g., a skeletal muscle cell. In some embodiments, the stem cells described herein (e.g., iPSCs) are gene-edited as described herein and then differentiated into a cell type of interest. In some embodiments, the differentiated cell retains the gene-edits of the cell from which it is derived.

The cells described herein may be differentiated into relevant cell types. In general, differentiation comprises maintaining the cells of interest for a period time and under conditions sufficient for the cells to differentiate into the differentiated cells of interest. For example, the engineered stem cells disclosed herein may be differentiated into mesenchymal progenitor cells (MPCs), hypoimmunogenic cardiomyocytes, muscle progenitor cells, blast cells, endothelial cells (ECs), macrophages, natural killer cells, hepatocytes, beta cells (e.g., pancreatic beta cells), pancreatic endoderm progenitors, pancreatic endocrine progenitors, or neural progenitor cells (NPCs). In some embodiments, any of the stem cells described herein are differentiated after gene-editing. In some embodiments, a cell is differentiated into a natural killer (NK) cell.

Stem cells are capable of both proliferation and giving rise to more progenitor cells, these in turn having the ability to generate a large number of mother cells that can in turn give rise to differentiated or differentiable daughter cells. The daughter cells themselves can be induced to proliferate and produce progeny that subsequently differentiate into one or more mature cell types, while also retaining one or more cells with parental developmental potential. The term “stem cell” refers then to a cell with the capacity or potential, under particular circumstances, to differentiate to a more specialized or differentiated phenotype, and which retains the capacity, under certain circumstances, to proliferate without substantially differentiating. In one aspect, the term progenitor or stem cell refers to a generalized mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell that itself is derived from a multipotent cell, and so on. While each of these multipotent cells may be considered stem cells, the range of cell types that each can give rise to may vary considerably. Some differentiated cells also have the capacity to give rise to cells of greater developmental potential. Such capacity can be natural or can be induced artificially upon treatment with various factors. In many biological instances, stem cells can also be “multipotent” because they can produce progeny of more than one distinct cell type, but this is not required for “stem-ness.”

A “differentiated cell” is a cell that has progressed further down the developmental pathway than the cell to which it is being compared. Thus, stem cells can differentiate into lineage-restricted precursor cells (such as a hematopoietic stem and progenitor cell (HSPC)), which in turn can differentiate into other types of precursor cells further down the pathway (such as a common lymphoid progenitor cell), and then to an end-stage differentiated cell, such as a natural killer cell, which plays a characteristic role in a certain tissue type, and may or may not retain the capacity to proliferate further.

In some embodiments, any of the gene-edited cells described herein have one of more of the following characteristics: increased persistency, immune evasiveness, lack of an alloimmune T cell response, increased cytotoxic activity, improved antibody-dependent cellular cytotoxicity (ADCC), or increased anti-tumor activity. In some embodiments, any of the gene-edited cells described herein have one of more of the following characteristics relative to an un-edited (wild-type) cell described herein; increased persistency, immune evasiveness, lack of an alloimmune T cell response, increased cytotoxic activity, improved antibody-dependent cellular cytotoxicity (ADCC), or increased anti-tumor activity. In some embodiments, any of the gene-edited cells described herein are capable of cell expansion in the absence of exogenous IL15.

### Embryonic Stem Cells

The cells described herein may be embryonic stem cells (ESCs). ESCs are derived from blastocysts or other structures of mammalian embryos and are able differentiate into any cell type and propagate rapidly. ESCs typically maintain high telomerase activity and exhibit remarkable long-term proliferative potential, making these cells excellent candidates for use as gene-edited stem cells. In some embodiments, ESCs with one or more of the following edits: IL15 knock-in and a CAR knock-in (e.g., an anti-GPR87 CAR knock-in), and FAS null, are differentiated into NK cells.

### Adult Stem Cells

The cells described herein may be adult stem cells (ASCs). ASCs are undifferentiated cells that may be found in mammals, e.g., humans. ASCs are defined by their ability to self-renew, e.g., be passaged through several rounds of cell replication while maintaining their undifferentiated state, and ability to differentiate into several distinct cell types, e.g., glial cells. Adult stem cells are a broad class of stem cells that may encompass hematopoietic stem cells, mammary stem cells, intestinal stem cells, mesenchymal stem cells, endothelial stem cells, neural stem cells, olfactory adult stem cells, neural crest stem cells, and testicular cells. In some embodiments, ASCs with one or more of the following edits: IL15 knock-in and a CAR knock-in (e.g., an anti-GPR87 CAR knock-in), and FAS null, are differentiated into NK cells.

### Induced Pluripotent Stem Cells

The cells described herein may be induced pluripotent stem cells (iPSCs). An iPSC may be generated directly from an adult human cell by introducing genes that encode critical transcription factors involved in pluripotency, e.g., Oct4, Sox2, cMyc, and Klf4. An iPSC may be derived from the same subject to which subsequent progenitor cells are to be administered. That is, a somatic cell can be obtained from a subject, reprogrammed to an induced pluripotent stem cell, and then re-differentiated into a progenitor cell to be administered to the subject (e.g., autologous cells). However, in the case of autologous cells, a risk of immune response and poor viability post-engraftment remain. In some embodiments, iPSC are generated from adult somatic cells using genetic reprogramming methods known in the art. In some embodiments, the iPSCs are derived from a commercial source. In some embodiments, the cells described herein are iPSCs or a derivative cell. In some embodiments, iPSC with one or more of the following edits: IL15 knock-in and a CAR knock-in (e.g., an anti-GPR87 CAR knock-in), and FAS null, are differentiated into NK cells.

### Mesoderm

The cells described herein can be mesodermal cells which is one of the three germinal layers in embryonic development. The mesoderm eventually differentiates into, but is not limited to muscle, connective tissue, bone, red blood cells, white blood cells, and microglia. In some embodiments, the gene-edited cells are mesodermal cells. In some embodiments, mesodermal cells are derived from any of the stem cells described herein. In some embodiments, mesodermal cells are derived from iPSC. In some embodiments, the mesodermal cells have any of the gene-edits described herein. In some embodiments, the mesodermal cells are differentiated into NK cells. In some embodiments, mesodermal cells with one or more of the following edits: an IL15/IL15Rα knock-in and a CAR knock-in (e.g., an anti-GPR87 CAR knock-in), and FAS null, are differentiated into NK cells.

### Hemogenic Endothelium

The cells described herein can be hemogenic endothelium (HE) cells which is an intermediate precursor of hematopoietic progenitors. In some embodiments, the cells are hemogenic endothelium cells. In some embodiments, the gene-edited cells described herein are hemogenic endothelium cells. In some embodiments, hemogenic endothelium cells are derived from any of the stem cells described herein. In some embodiments, hemogenic endothelium cells are derived from iPSC. In some embodiments, the hemogenic endothelial cells have any of the gene-edits described herein. In some embodiments, the hemogenic endothelial cells are differentiated into NK cells. In some embodiments, HE cells with one or more of the following edits: an IL15/IL15Rα knock-in and a CAR knock-in (e.g., an anti-GPR87 CAR knock-in), and FAS null, are differentiated into NK cells.

### Human Hematopoietic Stem and Progenitor Cells

The cells described herein can be human hematopoietic stem and progenitor cells (hHSPCs). This stem cell lineage gives rise to all blood cell types, including erythroid (erythrocytes or red blood cells (RBCs)), myeloid (monocytes and macrophages, neutrophils, basophils, eosinophils, megakaryocytes/platelets, and dendritic cells), and lymphoid (T-cells, B-cells, NK-cells). Blood cells are produced by the proliferation and differentiation of a very small population of pluripotent hematopoietic stem cells (HSCs) that also have the ability to replenish themselves by self-renewal. During differentiation, the progeny of HSCs progress through various intermediate maturational stages, generating multi-potential and lineage-committed progenitor cells prior to reaching maturity. Bone marrow (BM) is the major site of hematopoiesis in humans and, under normal conditions, only small numbers of hematopoietic stem and progenitor cells (HSPCs) can be found in the peripheral blood (PB). Treatment with cytokines, some myelosuppressive drugs used in cancer treatment, and compounds that disrupt the interaction between hematopoietic and BM stromal cells can rapidly mobilize large numbers of stem and progenitors into the circulation. In some embodiments, HSPCs are derived from any of the stem cells described herein. In some embodiments, HSPCs are derived from iPSCs. In some embodiments, the HSPCs have any of the gene-edits described herein. In some embodiments, the HSPCs cells are differentiated into NK cells. In some embodiments, HSPCs with one or more of the following edits: an IL15/IL15Rα knock-in and a CAR knock-in (e.g., an anti-GPR87 CAR knock-in), and FAS null, are differentiated into NK cells.

### Common Lymphoid Progenitor

The cells described herein can be common lymphoid progenitor (CLP) cells. CLPs are descendants of HSPCs. These cells differentiate into the lymphoid lineage of blood cells. Further differentiation yields B-cell progenitor cells, Natural Killer cells, and Thymocytes. In some embodiments, the cells described herein are common lymphoid progenitors. In some embodiments, the gene-edited cells described herein are common lymphoid progenitors. In some embodiments, CLP cells are derived from iPSCs. In some embodiments, the CLP cells have any of the gene-edits described herein. In some embodiments, the CLP cells are differentiated into NK cells. In some embodiments, CLP cells with one or more of the following edits: an IL15/IL15Rα knock-in and a CAR knock-in (e.g., an anti-GPR87 CAR knock-in), and FAS null, are differentiated into NK cells.

Differentiation of Cells into Other Cell Types

Another step of the methods of the present disclosure may comprise differentiating cells into differentiated cells. The differentiating step may be performed according to any method known in the art. For example, human iPSCs are differentiated into natural killer cells using methods known in the art. In some embodiments, the differentiating step may be performed according to Zhu and Kaufman, bioRxiv 2019; dx.doi.org/10.1101/614792. A differentiated cell may be any somatic cell of a mammal, e.g., a human. In some embodiments, a somatic cell may be an endocrine secretory epithelial cell (e.g., thyroid hormone secreting cells, adrenal cortical cells), an exocrine secretory epithelial cell (e.g., salivary gland mucous cell, prostate gland cell), a hormone-secreting cell (e.g., anterior pituitary cell, pancreatic islet cell), a keratinizing epithelial cell (e.g., epidermal keratinocyte), a wet stratified barrier epithelial cell, a sensory transducer cell (e.g., a photoreceptor), an autonomic neuron cells, a sense organ and peripheral neuron supporting cell (e.g., Schwann cell), a central nervous system neuron, a glial cell (e.g., astrocyte, oligodendrocyte), a lens cell, an adipocyte, a kidney cell, a barrier function cell (e.g., a duct cell), an extracellular matrix cell, a contractile cell (e.g., skeletal muscle cell, heart muscle cell, smooth muscle cell), a blood cell (e.g., erythrocyte), an immune system cell (e.g., megakaryocyte, microglial cell, neutrophil, mast cell, a T cell, a B cell, a Natural Killer cell), a germ cell (e.g., spermatid), a nurse cell, or an interstitial cell. In some embodiments, any of the stem cells described herein are differentiated into NK cells. In some embodiments, any of the derivative cell types described herein are differentiated into NK cells.

Provided herein, in some embodiments, are methods for generating Natural Killer (NK) cells from stem cells. The method includes: (a) culturing a population of stem cells in a first medium comprising a ROCK inhibitor under conditions sufficient to form aggregates; (b) culturing the aggregates in a second medium comprising BMP-4; (c) culturing the aggregates in a third medium comprising BMP-4, FGF2, a WNT pathway activator, and Activin A; (d) culturing the aggregates in a fourth medium comprising FGF2, VEGF, TPO, SCF, IL-3, FLT3L, WNT C-59 and an activin/nodal inhibitor to form a cell population comprising hematopoietic stem and progenitor cells (HSPCs); (e) culturing the cell population in a fifth medium comprising FGF2, VEGF, TPO, SCF, IL-3 and FLT3L; (f) culturing the cell population in a sixth medium comprising IL-3, IL-7, FLT3L, IL-15 and SCF; (g) culturing the cell population in a seventh medium comprising IL-7, FLT3L, IL-15 and SCF for a time sufficient to generate NK cells. In some embodiments, the method includes (a) culturing a population of stem cells in a first medium comprising a ROCK inhibitor under conditions sufficient to form aggregates; (b) culturing the aggregates in a second medium comprising BMP-4; (c) culturing the aggregates in a third medium comprising BMP-4, FGF2, a WNT pathway activator, and Activin A; (d) culturing the aggregates in a fourth medium comprising FGF2, VEGF, TPO, SCF, IL-3, FLT3L, and an activin/nodal inhibitor to form a cell population comprising hematopoietic stem and progenitor cells (HSPCs); (e) culturing the cell population in a fifth medium comprising FGF2, VEGF, TPO, SCF, IL-3 and FLT3L; (f) culturing the cell population in a sixth medium comprising IL-3, IL-7, FLT3L, IL-15 and SCF; (g) culturing the cell population in a seventh medium comprising IL-7, FLT3L, IL-15 and SCF and (h) culturing the cell population in an eighth medium comprising IL-7, FLT3L, IL-15, SCF and nicotinamide for a time sufficient to generate NK cells. In some embodiments, the second medium further includes a ROCK inhibitor. In some embodiments, the ROCK inhibitor is thiazovivin. In some embodiments, the WNT pathway activator is CHIR-99021. In some embodiments, the activin/nodal inhibitor is SB-431542.

In some embodiments, the method includes (a) culturing a population of stem cells in a first medium comprising a ROCK inhibitor under conditions sufficient to form aggregates; (b) culturing the aggregates in a second medium comprising BMP-4; (c) culturing the aggregates in a third medium comprising BMP-4, FGF2, a WNT pathway activator, and Activin A; (d) culturing the aggregates in a fourth medium comprising FGF2, VEGF, TPO, SCF, IL-3, FLT3L, and an activin/nodal inhibitor to form a cell population comprising HSPCs; (e) culturing the cell population in a fifth medium comprising FGF2, VEGF, TPO, SCF, IL-3 and FLT3L; (f) culturing the cell population in a sixth medium comprising IL-3, IL-7, FLT3L, IL-15 and SCF; (g) culturing the cell population in a seventh medium comprising IL-7, FLT3L, IL-15 and SCF and (h) culturing the cell population in an eighth medium comprising IL-7, FLT3L, IL-15, and SCF for a time sufficient to generate NK cells. In some embodiments, the method includes (a) culturing a population of stem cells in a first medium comprising a ROCK inhibitor under conditions sufficient to form aggregates; (b) culturing the aggregates in a second medium comprising BMP-4; (c) culturing the aggregates in a third medium comprising BMP-4, FGF2, a WNT pathway activator, and Activin A; (d) culturing the aggregates in a fourth medium comprising FGF2, VEGF, TPO and SCF to form a cell population comprising hematopoietic stem and progenitor cells (HSPCs); (e) culturing the cell population in a fifth medium comprising FGF2, VEGF, TPO, SCF, IL-3 and FLT3L; (f) culturing the cell population in a sixth medium comprising IL-3, IL-7, FLT3L, IL-15 and SCF; (g) culturing the cell population in a seventh medium comprising IL-7, FLT3L, IL-15 and SCF and (h) culturing the cell population in an eighth medium comprising IL-7, FLT3L, IL-15, and SCF for a time sufficient to generate NK cells. In some embodiments, the ROCK inhibitor is thiazovivin. In some embodiments, the ROCK inhibitor is Y27632. In some embodiments, the WNT pathway activator is CHIR-99021. In some embodiments, the activin/nodal inhibitor is SB-431542.

In some embodiments, steps (a)-(g) occurs between 20-35 days. In some embodiments, step (a) includes culturing for 12-48 hours. In some embodiments, step (b) includes culturing for up to 24 hours. In some embodiments, step (c) includes culturing for 1-3 days. In some embodiments, step (d) includes culturing for 1-3 days. In some embodiments, step (e) includes culturing for 1-3 days. In some embodiments, step (f) includes culturing for up to 7 days. In some embodiments, step (g) includes culturing for at least 6 days and up to 21-28 days total. In some embodiments, step (a) includes culturing for 16-20 hours; step (b) includes culturing for 6-10 hours; step (c) includes culturing for 2 days; step (d) includes culturing for 2 days; step (e) includes culturing for 2 days; step (f) includes culturing for 4 days; and/or step (g) includes culturing for 14-28 days.

In some embodiments, steps (a)-(h) occur between 24 and 36 days. In some embodiments, step (a) includes culturing for 12-48 hours. In some embodiments, step (b) includes culturing for up to 24 hours. In some embodiments, step (c) includes culturing for 1-3 days. In some embodiments, step (d) includes culturing for 1-3 days. In some embodiments, step (e) includes culturing for 1-3 days. In some embodiments, step (f) includes culturing for up to 7 days. In some embodiments, step (g) includes culturing for up to 6 days. In some embodiments, step (h) includes culturing for at least 6 days and up to 10-16 days total. In some embodiments, step (a) includes culturing for 16-20 hours; step (b) includes culturing for 6-10 hours; step (c) includes culturing for 2 days; step (d) includes culturing for 2 days; step (e) includes culturing for 2 days; step (f) includes culturing for 4 days; step (g) includes culturing for 6 days and/or step (h) includes culturing for 10-16 days.

In some embodiments, steps (a)-(h) occurs between 24 and 36 days. In some embodiments, step (a) includes culturing for 12-48 hours. In some embodiments, step (b) includes culturing for up to 24 hours. In some embodiments, step (c) includes culturing for 1-3 days. In some embodiments, step (d) includes culturing for 1-3 days. In some embodiments, step (e) includes culturing for 2-6 days. In some embodiments, step (f) includes culturing for up to 7 days. In some embodiments, step (g) includes culturing for up to 6 days. In some embodiments, step (h) includes culturing for at least 6 days and up to 10-16 days total. In some embodiments, step (a) includes culturing for 16-20 hours; step (b) includes culturing for 6-10 hours; step (c) includes culturing for 2 days; step (d) includes culturing for 2 days; step (e) includes culturing for 6 days; step (f) includes culturing for 4 days; step (g) includes culturing for 6 days and/or step (h) includes culturing for 10-17 days.

In some embodiments, the method is carried out under suspension agitation. In some embodiments, the suspension agitation includes rotation. In some embodiments, the first and second media include StemFlex medium. In some embodiments, the third, fourth and fifth media include APEL medium. In some embodiments, the sixth medium can include APEL medium. In some embodiments, the sixth and seventh media can include DMEM/F12 medium. In some aspects, the sixth and seventh media comprise DMEM (high glucose)/F12 medium. In some embodiments, the sixth and seventh media include human serum (e.g., at the concentration of 10-20%), zinc sulfate (e.g., at a concentration of about 20-40 μM), ethanolamine (e.g., at a concentration of about 10-100 μM), b-mercaptoethanol (e.g., at a concentration of about 0.1-5 μM), glucose (e.g., at a total concentration of 2-40 mM), or any combination thereof. In some embodiments, the sixth and seventh media include human serum (e.g., at the concentration of 15%), zinc sulfate (e.g., at a concentration of about 36 or 37 μM), ethanolamine (e.g., at a concentration of about 50 μM), b-mercaptoethanol (e.g., at a concentration of about 1 μM), glucose (e.g., at a total concentration of 27 mM), or any combination thereof. In some embodiments, the sixth and seventh media include human serum (e.g., at a concentration of about 10-40%), zinc sulfate (e.g., at a concentration of about 20-40 μM), ethanolamine (e.g., at a concentration of about 10-100 μM), glucose (e.g., at a total concentration of about 2-40 mM), or any combination thereof. In some embodiments, the sixth and seventh media include human serum (e.g., at a concentration of about 20%), zinc sulfate (e.g., at a concentration of about 37 μM), ethanolamine (e.g., at a concentration of about 50 μM), glucose (e.g., at a total concentration of about 20 mM), or any combination thereof. In some embodiments, the eighth media includes human serum (e.g., at a concentration of about 2-15%), zinc sulfate (e.g., at a concentration of about 20-40 μM), ethanolamine (e.g., at a concentration of about 10-100 μM), glucose (e.g., at a total concentration of about 2-40 mM), or any combination thereof. In some embodiments, the eighth media can include DMEM/F12 medium. In some aspects, the eighth media comprises DMEM (high glucose)/F12 medium. In some embodiments, the eighth media includes human serum (e.g., at a concentration of about 10%), zinc sulfate (e.g., at a concentration of about 37 μM), ethanolamine (e.g., at a concentration of about 50 μM), glucose (e.g., at a total concentration of about 20 mM), or any combination thereof. In any of the sixth, seventh, and eighth media provided herein, the total glucose concentration comprises glucose from all sources including glucose present in the base media and any added glucose. In each of the sixth, seventh, and eighth media provided herein, additional glucose may be added to a glucose containing base media (e.g., DMEM, F12 or DMEM (high glucose)/F12 medium) to reach the “total” glucose concentration. In some embodiments, about 10.25 mM of glucose is added to the base media of the sixth or seventh media to reach the total glucose concentration of about 27 mM. In some embodiments, about 4.66 mM of glucose is added to the base media of the sixth or seventh media to reach the total glucose concentration of about 20 mM. In some embodiments, about 2.33 mM of glucose is added to the base media of the eighth media to reach the total glucose concentration of about 20 mM. In some embodiments, the first medium includes 10 μM of the ROCK inhibitor. In some embodiments, the first medium includes 5 μM of the ROCK inhibitor. In some embodiments, the second medium includes 30 ng/mL BMP-4 and 10 μM of a ROCK inhibitor. In some embodiments, the third medium includes 30 ng/mL BMP-4, 100 ng/mL FGF2, 6 μM CHIR-99021, and 2.5-5 ng/mL Activin A. In some embodiments, the third medium includes 30 ng/mL BMP-4, 100 ng/mL FGF2, 7 μM CHIR-99021, and 2.5-5 ng/mL Activin A. In some embodiments, the third medium includes 30 ng/mL BMP-4, 100 ng/mL FGF2, 3-3.5 μM CHIR-99021, and 2.5 ng/mL Activin A.

In some embodiments, half of the third medium is added to the stem cell aggregates. In some embodiments, the fourth and fifth media include 20 ng/mL FGF, 20 ng/mL VEGF, 20 ng/mL TPO, 100 ng/mL SCF, 40 ng/mL IL-3, and 10-20 ng/mL FLT3L. In some embodiments, the fourth medium further includes 2 μM WNT C-59 and 5 μM SB-431542. In some embodiments, the fourth medium further includes 5 μM SB-431542. In some embodiments, the fourth medium does not include WNT C-59. In some embodiments, the fourth media includes 20 ng/mL FGF, 20 ng/mL VEGF, 20 ng/mL TPO, and 40 ng/mL SCF, In some embodiments, the fourth media does not comprise IL-3, FLT3L, or SB-431542. In some embodiments, the sixth and seventh media includes 20 ng/mL IL-7, 10-20 ng/mL FLT3L, 10-20 ng/mL IL-15, and 20 ng/mL SCF. In some embodiments, the sixth medium includes 5 ng/mL IL-3. In some embodiments, the eighth media includes IL-7, FLT3L, IL-15, SCF and nicotinamide. In various embodiments, the eighth medium includes 10-20 ng/mL IL-7, 5-20 ng/mL FLT3L, 10-30 ng/mL IL-15, 20-40 ng/mL SCF, and 1-15 mM nicotinamide. In various embodiments, the eighth medium includes 10 ng/mL IL-7, 7.5 ng/mL FLT3L, 15 ng/mL IL-15, 20 ng/mL SCF and 6.5 mM nicotinamide. In some embodiments, the eighth media includes IL-7, FLT3L, IL-15, and SCF. In various embodiments, the eighth medium includes 10-20 ng/mL IL-7, 5-20 ng/mL FLT3L, 10-30 ng/mL IL-15, and 20-40 ng/mL SCF. In various embodiments, the eighth medium includes 10 ng/mL IL-7, 7.5 ng/mL FLT3L, 15 ng/mL IL-15, and 20 ng/mL SCF.

In some embodiments, the HSPCs of step (d) express CD34. In some embodiments, the NK cells express CD56. In some embodiments, the NK cells express at least one activating receptor. In some embodiments, the at least one activating receptor is selected from NKp44, NKp46, CD16, KIR2DL4, and any combination thereof. In some embodiments, the NK cells express at least one inhibitory receptor. In some embodiments, the at least one inhibitory receptor is selected from CD94, NKG2A, KIR3DL2, and any combination thereof.

In some embodiments, the NK cells include at least one function associated with endogenous NK cells. In some embodiments, the at least one function includes the ability to induce cell lysis and cell death of a target cell. In some embodiments, the at least one function includes degranulation. In some embodiments, the degranulation includes release of perforin and granzyme B. The degranulation can include expression of CD107a on the cell surface of an NK cell.

In some embodiments, the population of stem cells is a population of engineered cells, such as the engineered cells generated by the methods disclosed herein. In some embodiments, the population of engineered cells is differentiated by the methods of generating Natural Killer (NK) cells from stem cells disclosed herein. In some embodiments, a plurality of Natural Killer (NK) cells is generated by the method of generating Natural Killer (NK) cells from stem cells disclosed herein. Also disclosed herein is a plurality of NK cells is for use in treating a subject in need thereof. In some embodiments, the subject is a human who has, is suspected of having, or is at risk for a cancer. Also disclosed herein is a method comprising administering to a subject the plurality of NK cells.

### Natural Killer Cells

Natural killer (NK) cells are a subpopulation of lymphocytes which play a critical role in the innate immune system. NK cells have cytotoxicity against a variety of cells including but not limited to tumor cells and virus-infected cells. In some embodiments, the stem cells described herein are differentiated to Natural Killer cells. In some embodiments, iPSCs are differentiated into NK cells. In some embodiments, the engineered NK cells (such as cells derived from gene-edited iPSCs by differentiation, e.g., iNK cells) have enhanced anti-tumor activity as compared to un-edited or wild type NK cells. In some embodiments, anti-tumor activity of the engineered NK cells is increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, or at least 90% relative to control (e.g., un-edited or wild type) NK cells.

In some embodiments, the engineered NK cells exhibit increased cellular lysis capability relative to control cells. In some embodiments, the engineered NK cells of the present disclosure exhibit at least 10% increase in cellular lysis capability (kill at least 10% more target cells), or at least 20% increase in cellular lysis capability (kill at least 20% more target cells), relative to control (e.g., un-edited or wild type) cells. For example, the engineered NK cells of the present disclosure may exhibit an at least at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, or at least 90% increase in cellular lysis capability, relative to control (e.g., un-edited or wild type) cells. In some embodiments, the engineered NK cells of the present disclosure exhibit a 20%-100%, 20%-90%, 20%-80%, 20%-70%, 20%-60%, 20%-50%, 30%-100%, 30%-90%, 30%-80%, 30%-70%, 30%-60%, 30%-50%, 40%-100%, 40%-90%, 40%-80%, 40%-70%, 40%-60%, 40%-50, 50%-100%, 50%-90%, 50%-80%, 50%-70%, or 50%-60% increase in cellular lysis capability, relative to control (e.g., un-edited or wild type) cells. In some embodiments, the target cells are T cells. In some embodiments, the target cells are cancer cells. In some embodiments, the target cells are leukemia cells. In some embodiments, the target cells are liver cancer cells. In some embodiments, the target cells are lung cancer cells. In some embodiments, this increase in cellular lysis capability is observed at E:T (effector:target cell) ratio of at or about 0.1:1. In some embodiments, this increase in cellular lysis capability is observed at E:T (effector:target cell) ratio of at or about 0.5:1. In some embodiments, this increase in cellular lysis capability is observed at E:T (effector:target cell) ratio of at or about 1:1. In some embodiments, this increase in cellular lysis capability is observed at E:T (effector:target cell) ratio of at or about 4:1. In some embodiments, this increase in cellular lysis capability is observed at E:T (effector:target cell) ratio of at or about 0.1:1, when the target cell is K562 and when the cells are co-cultured for, e.g., 24 hours. In some embodiments, this increase in cellular lysis capability is observed at E:T (effector:target cell) ratio of at or about 0.5:1, when the target cell is K562 and when the cells are co-cultured for, e.g., 24 hours. In some embodiments, this increase in cellular lysis capability is observed at E:T (effector:target cell) ratio of at or about 1:1, when the target cell is K562 and when the cells are co-cultured for, e.g., 24 hours. In some embodiments, this increase in cellular lysis capability is observed at E:T (effector:target cell) ratio of at or about 0.1:1, when the target cell is RPMI and when the cells are co-cultured for, e.g., 24 hours. In some embodiments, this increase in cellular lysis capability is observed at E:T (effector:target cell) ratio of at or about 0.5:1, when the target cell is RPMI and when the cells are co-cultured for, e.g., 24 hours. In some embodiments, this increase in cellular lysis capability is observed at E:T (effector:target cell) ratio of at or about 1:1, when the target cell is RPMI and when the cells are co-cultured for, e.g., 24 hours. In some embodiments, this increase in cellular lysis capability is observed at E:T (effector:target cell) ratio of at or about 0.1:1, when the target cell is PC-9 and when the cells are co-cultured for, e.g., 24 hours. In some embodiments, this increase in cellular lysis capability is observed at E:T (effector:target cell) ratio of at or about 0.5:1, when the target cell is PC-9 and when the cells are co-cultured for, e.g., 24 hours. In some embodiments, this increase in cellular lysis capability is observed at E:T (effector:target cell) ratio of at or about 1:1, when the target cell is PC-9 and when the cells are co-cultured for, e.g., 24 hours. In some embodiments, this increase in cellular lysis capability is observed at E:T (effector:target cell) ratio of at or about 0.1:1, when the target cell is HepG2 and when the cells are co-cultured for, e.g., 24 hours. In some embodiments, this increase in cellular lysis capability is observed at E:T (effector:target cell) ratio of at or about 0.5:1, when the target cell is HepG2 and when the cells are co-cultured for, e.g., 24 hours. In some embodiments, this increase in cellular lysis capability is observed at E:T (effector:target cell) ratio of at or about 1:1, when the target cell is HepG2 and the cells are co-cultured for, e.g., 24 hours. In some embodiments, this increase in cellular lysis capability is observed at E:T (effector:target cell) ratio of at or about 0.1:1, when the target cell is L540 and when the cells are co-cultured for, e.g., 24 hours. In some embodiments, this increase in cellular lysis capability is observed at E:T (effector:target cell) ratio of at or about 0.5:1, when the target cell is L540 and when the cells are co-cultured for, e.g., 24 hours. In some embodiments, this increase in cellular lysis capability is observed at E:T (effector:target cell) ratio of at or about 1:1, when the target cell is L540 and the cells are co-cultured for, e.g., 24 hours.

In some embodiments, the population of cells comprising human NK cells has at least one of the following characteristics, or any combination thereof: (i) an alloimmune T cell reaction of less than 10% (e.g., less than 0.01%, 0.1%, 1%, 2%, 3%, 4%, 5%, 6%, 7%, 8%, 9%, 10%) relative to a population of unmodified human NK cells, (ii) cytotoxic activity resulting in killing more than 50% (e.g., 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) of target cells when the population of cells comprising human NK cells are mixed with the target cells at the ratio of 1:1, and (iii) at least 50% (e.g., 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) increase in cellular viability relative to a population of unmodified human NK cells.

In some embodiments, the population of cells comprising human NK cells has at least one of the following characteristics, or any combination thereof: (i) improved persistency, (ii) improved immune evasiveness, (iii) improved cytotoxic activity, (iv) improved antibody-dependent cellular cytotoxicity (ADCC) activity, and (v) improved anti-tumor activity; wherein the characteristics are improved relative to a population of unmodified human NK cells. In some embodiments, the population of cells comprising human NK cells, when co-cultured in vitro with a population of cancer cells, induce cell death of at least 60%, at least 70%, at least 80%, or at least 90% of the population of cancer cells after about 24 hours of co-culture. In some embodiments, the population of cells comprising human NK cells, when co-cultured in vitro with a population of cancer cells, induce cell death of at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, of the population of cancer cells after about 24 hours of co-culture.

In some embodiments, the engineered NK cells (and/or e.g., a population of cells comprising the engineered NK cells) express at least one, two, three, four, five, six, seven, eight or all of the following markers: CD45, CD56, CD94, NKG2A, CD16, NKp44, NKp46, KIR2DL4, and KIR3DL2, and optionally wherein the markers are expressed at least at 25%, 30%, 40%, 50%, 75%, 80%, 90%, 95% or 100% level or more relative to their expression in un-edited or wild type NK cells. In some embodiments, the engineered NK cells expresses at least one, two, three, four, five or all of the following markers: CD56, NKp44, NKp46, CD94, NKG2A and KIR2DL4, and optionally wherein the markers are expressed at least at 25%, 30%, 40%, 50%, 75%, 80%, 90%, 95% or 100% level or more relative to their expression in un-edited or wild type NK cells. In some embodiments, the engineered NK cells have at least 25%, at least 30%, at least 40%, at least 50%, at least 60%, at least 70%, at least 80%, at least 90%, at least 95% or at least 99% of the cell population expressing one, two, three, four, five, six, seven, eight or all of the following markers: CD45, CD56, CD94, NKG2A, CD16, NKp44, NKp46, KIR2DL4, and KIR3DL2. In some embodiments, the engineered NK cells have at least 25%, at least 30%, at least 40%, at least 50%, at least 60%, at least 70%, at least 80%, at least 90%, at least 95% or at least 99% of the cell population expressing one, two, three, four, five or all of the following markers: CD56, NKp44, NKp46, CD94, NKG2A and KIR2DL4.

In some embodiments, the engineered NK cells express at least one, two, three or all of the following markers: CD38, CD96, DNAM-1, and ICAM-1, and optionally wherein the markers are expressed at least at 25%, 30%, 40%, 50%, 75%, 80%, 90%, 95% or 100% level or more relative to their expression in un-edited or wild type NK cells. In some embodiments, the engineered NK cells express at least one, two, three or all of the following markers: CD38, CD96, DNAM-1, and ICAM-1, and optionally wherein the markers are expressed at least at 25%, 30%, 40%, 50%, 75%, 80%, 90%, 95% or 100% level or more relative to their expression in un-edited or wild type NK cells. In some embodiments, the engineered NK cells have at least 25%, at least 30%, at least 40%, at least 50%, at least 60%, at least 70%, at least 80%, at least 90%, at least 95% or at least 99% of the cell population expressing one, two, three or all of the following markers: CD38, CD96, DNAM-1, and ICAM-1. In some embodiments, the engineered NK cells have at least 25%, at least 30%, at least 40%, at least 50%, at least 60%, at least 70%, at least 80%, at least 90%, at least 95% or at least 99% of the cell population expressing one, two, three or all of the following markers: CD38, CD96, DNAM-1, and ICAM-1.

In some embodiments, the engineered NK cells express at least one, two, three or all of the following markers: NKG2D, TIM3, CD16, and CD25, and optionally wherein the markers are expressed at least at 25%, 30%, 40%, 50%, 75%, 80%, 90%, 95% or 100% level or more relative to their expression in un-edited or wild type NK cells. In some embodiments, the engineered NK cells express at least one, two, three or all of the following markers: NKG2D, TIM3, CD16, and CD25, and optionally wherein the markers are expressed at least at 25%, 30%, 40%, 50%, 75%, 80%, 90%, 95% or 100% level or more relative to their expression in un-edited or wild type NK cells. In some embodiments, the engineered NK cells have at least 10%, at least 15%, at least 20%, at least 25%, at least 30%, at least 40%, at least 50%, at least 60%, at least 70%, at least 80%, at least 90%, at least 95% or at least 99% of the cell population expressing one, two, three or all of the following markers: NKG2D, TIM3, CD16, and CD25. In some embodiments, the engineered NK cells have at least 10%, at least 15%, at least 20%, at least 25%, at least 30%, at least 40%, at least 50%, at least 60%, at least 70%, at least 80%, at least 90%, at least 95% or at least 99% of the cell population expressing one, two, three or all of the following markers: NKG2D, TIM3, CD16, and CD25.

The engineered human NK cells can express at least one, two, three, four or five of the markers of CD56, NKp44, NKp46, CD94, NKG2A, KIR2DL4, and a CAR. The at least one, two, three, four or five markers can be expressed in at least 25%, 30%, 40%, 50%, or 75% of the population of cells (e.g., in 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values of the population of cells). In some embodiments, the CAR is detected by binding to (e.g., staining with) Protein L. Protein L is a peptide isolated from bacteria which can be used to detect light chains of antibodies.

In some embodiments, the engineered NK cells of the present disclosure exhibit an increased cytokine secretion relative to control (e.g., un-edited or wild type) cells. In some embodiments, the engineered NK cells of the present disclosure exhibit about the same cytokine secretion level relative to control (e.g., un-edited or wild type) cells. In some embodiments, the engineered NK cells of the present disclosure exhibit a reduced (e.g., reduced by less than 10%, less than 20%, less than 30%, less than 40%, or less than 50%) cytokine secretion level relative to control (e.g., un-edited or wild type) cells. In some embodiments, the engineered NK cells of the present disclosure exhibit a reduced (e.g., reduced by more than 20%, more than 30%, more than 40%, more than 50%, or more than 75%) cytokine secretion level relative to control (e.g., un-edited or wild type) cells. In some embodiments, the engineered NK cells of the present disclosure exhibit an increased (e.g., increased by at least 5%, at least 10%, at least 20%, at least 30%, at least 40%, at least 50%, or at least 75%) cytokine secretion level relative to control (e.g., un-edited or wild type) cells. The cytokine(s) being measured can be, without limitation any one or more of: TNFα, IFNγ and IL-7. In some embodiments, the level of cytokines (e.g., TNFα, IFNγ and IL-7) secreted by the engineered NK cells is about the same as the level in control (e.g., un-edited or wild type) cells, when cells are co-cultured with target cells at the E:T ratio of or about 0.1:1. In some embodiments, the level of cytokines (e.g., TNFα, IFNγ and IL-7) secreted by the engineered NK cells is reduced (by, e.g., at least 10%, 20%, 30%, 40%, 50%, 60% or 70%, and/or no more than 50%, 60%, 70%, 80%, or 90%) relative to the level in control (e.g., un-edited or wild type) cells, when cells are co-cultured with target cells at the E:T ratio of or about 0.1:1. In some embodiments, the level of cytokines (e.g., TNFα, IFNγ and IL-7) secreted by the engineered NK cells is increased (by, e.g., at least 5%, 10%, 20%, 30%, 40%, 50%, 60% or 70%) relative to the level in control (e.g., un-edited or wild type) cells, when cells are co-cultured with target cells at the E:T ratio of or about 0.1:1.

In some embodiments, the engineered NK cells of the present disclosure exhibit an increased expression or release of Granzyme B or perforin relative to control (e.g., un-edited or wild type) cells. In some embodiments, the engineered NK cells of the present disclosure exhibit about the same expression or release level of Granzyme B or perforin relative to control (e.g., un-edited or wild type) cells. In some embodiments, the engineered NK cells of the present disclosure exhibit a reduced (e.g., reduced by less than 10%, less than 20%, less than 30%, less than 40%, or less than 50%) Granzyme B or perforin expression or release level relative to control (e.g., un-edited or wild type) cells. In some embodiments, the engineered NK cells of the present disclosure exhibit a reduced (e.g., reduced by more than 20%, more than 30%, more than 40%, more than 50%, or more than 75%) Granzyme B or perforin expression or release level relative to control (e.g., un-edited or wild type) cells. In some embodiments, the engineered NK cells of the present disclosure exhibit an increased (e.g., increased by at least 5%, at least 10%, at least 20%, at least 30%, at least 40%, at least 50%, or at least 75%) Granzyme B or perforin expression or release level relative to control (e.g., un-edited or wild type) cells. In some embodiments, the level of Granzyme B or perforin secreted by the engineered NK cells is about the same as the level in control (e.g., un-edited or wild type) cells, when cells are co-cultured with target cells at the E:T ratio of or about 0.1:1. In some embodiments, the level of Granzyme B or perforin secreted by the engineered NK cells is reduced (by, e.g., at least 10%, 20%, 30%, 40%, 50%, 60% or 70%, and/or no more than 50%, 60%, 70%, 80%, or 90%) relative to the level in control (e.g., un-edited or wild type) cells, when cells are co-cultured with target cells at the E:T ratio of or about 0.1:1. In some embodiments, the level of Granzyme B or perforin secreted by the engineered NK cells is increased (by, e.g., at least 5%, 10%, 20%, 30%, 40%, 50%, 60% or 70%) relative to the level in control (e.g., un-edited or wild type) cells, when cells are co-cultured with target cells at the E:T ratio of or about 0.1:1.

In some embodiments, the engineered NK cells of the present disclosure exhibit an increased (e.g., increased by at least 5%, at least 10%, at least 20%, at least 30%, at least 40%, at least 50%, or at least 75%) expression level of CD107a relative to control (e.g., un-edited or wild type) cells. In some embodiments, the engineered NK cells of the present disclosure exhibit about the same expression level of CD107a relative to control (e.g., un-edited or wild type) cells. In some embodiments, engineered NK cells of the present disclosure exhibit a reduced (e.g., reduced by less than 10%, less than 20%, less than 30%, less than 40%, or less than 50%) CD107a expression level relative to control (e.g., un-edited or wild type) cells. In some embodiments, the engineered NK cells of the present disclosure exhibit a reduced (e.g., reduced by more than 20%, more than 30%, more than 40%, more than 50%, or more than 75%) CD107a expression level relative to control (e.g., un-edited or wild type) cells.

In some embodiments, the engineered NK cells have higher proliferative capacity as compared to un-edited or wild-type NK cells. In some embodiments, the engineered NK cells have approximately the same proliferative capacity compared to un-edited or wild-type NK cells. In some embodiments, the engineered NK cells do not exhibit exhaustion or exhibit a low level of exhaustion (e.g., a level of exhaustion markers associated with a functional NK cell). In some embodiments, exhaustion is detected by detecting a reduced expression of IFNγ, granzyme B, perforin, CD107a, and/or TNFα in cells. In some embodiments, exhaustion is detected by detecting increased expression (e.g., on the surface of the cell) of an exhaustion marker, e.g., PD-1, LAG-3, TIGIT and/or TIM-3. In some embodiments, the engineered NK cells have normal or higher than normal expression of perforin, granzyme B, CD107a, IFNγ and/or TNFα (relative to un-edited or wild-type cells). In some embodiments, the engineered NK cells have lower than normal or no expression of PD-1, LAG-3, TIGIT and/or TIM-3 (relative to un-edited or wild-type cells). In some embodiments, engineered NK cells of the present disclosure exhibit reduced exhaustion, relative to control (e.g., un-edited cells or wild type) NK cells.

In some embodiments, the engineered NK cells of the present disclosure exhibit about the same cellular viability as control (e.g., un-edited or wild-type) cells. In some embodiments, the engineered NK cells of the present disclosure exhibit increased cellular viability relative to control (e.g., un-edited or wild-type) cells. In some embodiments, the engineered NK cells of the present disclosure exhibit at least 10% or at least 20% increase in cellular viability, relative to control cells. For example, the engineered NK cells of the present disclosure may exhibit at least 25%, at least 30%, at least 35%, at least 40%, at least 45%, at least 50%, at least 55%, at least 60%, at least 65%, at least 70%, at least 75%, at least 80%, or at least 90% increase in cellular viability, relative to control cells. In some embodiments, the engineered NK cells of the present disclosure exhibit a 20%-100%, 20%-90%, 20%-80%, 20%-70%, 20%-60%, 20%-50%, 30%-100%, 30%-90%, 30%-80%, 30%-70%, 30%-60%, 30%-50%, 40%-100%, 40%-90%, 40%-80%, 40%-70%, 40%-60%, 40%-50%, 50%-100%, 50%-90%, 50%-80%, 50%-70%, or 50%-60% increase in cellular viability, relative to control cells. Methods of measuring cell viability are known to those of skill in the art and described herein.

In some embodiments, the engineered NK cells have higher expression of one or more cell cycle genes, one or more cell division genes, and/or one or more DNA replication genes, as compared to un-edited or wild type NK cells. In some embodiments, the engineered NK cells have approximately the same expression of one or more cell cycle genes, one or more cell division genes, and/or one or more DNA replication genes, as compared to un-edited or wild type NK cells.

In some embodiments, gene-edited iPSC cells are differentiated into NK cell having any of the characteristics described herein. In some embodiments, iPSC cells are edited with FAS null, IL15/IL15Rα knock-in, CAR KI (e.g., an anti-GPC3 CAR KI, an anti-GPR87 CAR knock-in), and/or NKG2D knock-in, then differentiated into NK cells. In some embodiments, iPSC cells are edited with CISH null, FAS null, IL15/IL15Rα knock-in, an anti-GPR87 CAR knock-in and/or NKG2D knock-in, then differentiated into NK cells. In some embodiments, iPSC cells are edited with FAS null, IL15/IL15Rα knock-in, and an anti-GPR87 CAR knock-in, then differentiated into NK cells.

In some embodiments, any of the engineered NK cells described herein have one of more of the following characteristics relative to an un-edited (wild-type) NK cell described herein: increased persistency, increased immune evasiveness, lack of an alloimmune T cell response, increased cytotoxic activity, improved antibody-dependent cellular cytotoxicity (ADCC), or increased anti-tumor activity.

In some embodiments, persistence of the engineered cells is assessed by analyzing their presence and quantity in one or more tissue samples that are collected from a subject following administration of the engineered cells to the subject. In some embodiments, persistence is defined as the longest duration of time from administration to a time wherein a detectable level of the engineered cells is present in a given tissue type (e.g., peripheral blood). In some embodiments, persistence is defined as the continued absence of disease (e.g., complete response or partial response). Determination of the absence of disease and response to treatment are known to those of skill in the art and described herein.

Methods of appropriate tissue collection, preparation, and storage are known to one skilled in the art. In some embodiments, persistence of cells is assessed in one or more tissue samples from a group comprised of peripheral blood, cerebrospinal fluid, tumor, skin, bone, bone marrow, breast, kidney, liver, lung, lymph node, spleen, gastrointestinal tract, tonsils, thymus and prostate. In some embodiments, a quantity of cells is measured in a single type of tissue sample (e.g., peripheral blood). In some embodiments, a quantity of cells is measured in multiple tissue types (e.g., peripheral blood in addition to bone marrow and cerebrospinal fluid). By measuring quantity of cells in multiple tissue types, the distribution of cells throughout different tissues of the body can be determined. In some embodiments, a quantity of cells is measured in one or more tissue samples at a single time point following administration. In some embodiments, a quantity of cells is measured in one or more tissue samples at multiple time points following administration.

A detectable level of the engineered cells in a given tissue can be measured by known methodologies. Methods for assessing the presence or quantity of cells in a tissue of interest are known to those of skill in the art. Such methods include, but are not limited to, reverse transcription polymerase chain reaction (RT-PCR), competitive RT-PCR, real-time RT-PCR, RNase protection assay (RPA), quantitative immunofluorescence (QIF), flow cytometry, northern blotting, nucleic acid microarray using DNA, western blotting, enzyme-linked immunosorbent assay (ELISA), radioimmunoassay (RIA), tissue immunostaining, immunoprecipitation assay, complement fixation assay, fluorescence-activated cell sorting (FACS), mass spectrometry, magnetic bead-antibody immunoprecipitation, or protein chip.

As used herein, in some embodiments, persistence is the longest period from the time of administration to a time wherein a detectable level of the engineered cells is measured. In some embodiments, a detectable level of cells is defined in terms of the limit of detection of a method of analysis. The limit of detection can be defined as the lowest quantity of a component or substance that can be reliably and reproducibly measured by an analytical procedure when compared to a tissue sample expected to have no quantity of the component or substance of interest. A non-limiting exemplary method to determine a reproducible limit of detection is to measure the analytical signal for replicates of a zero calibrator relative to a blank sample (Armbruster, D. et al. (2008) Clin Biochem Rev. 29:S49-S52). A blank sample is known to be devoid of an analyte of interest. A zero calibrator is the highest dilution of a test sample of known concentration or quantity that gives analytical signal above that measured for the blank sample. By quantifying the analytical signal for at least 5, 6, 7, 8, 9, 10, 11, 12, 13, 14, 15, 16, 17, 18, 19, 20, 21, 22, 23, 24, 25, 26, 27, 28, 29, or 30 replicates of a zero calibrator, one can determine an average and standard deviation (SD) for the limit of detection of an analytical method of interest. Selection of a method with a suitable limit of detection for quantifying donor T cells in a given tissue can be ascertained by one skilled in the art. In some embodiments, a detectable level of cells is any quantity of cells in a tissue sample that gives an analytical signal above the limit of detection for a method of analysis. In some embodiments, a detectable level of cells is any quantity of cells in a tissue sample that gives an analytical signal that is at least 2 SDs, 3 SDs, 4 SDs, 5 SDs, 6 SDs, 7 SDs, 8 SDs, 9 SDs, or 10 SDs, above the limit of detection for the method of analysis.

It is known that CAR-expressing donor cells can undergo expansion following administration to a recipient. Expansion is a response to antigen recognition and signal activation (Savoldo, B. et al. (2011) J Clin Invest. 121:1822; van der Stegen, S. et al. (2015) Nat Rev Drug Discov. 14:499-509). In some embodiments, following expansion, CAR-expressing engineered cells undergo a contraction period, wherein a portion of the cell population that are short-lived effector cells are eliminated and what remains is a portion of the cell population that are long-lived memory cells. In some embodiments, persistence is a measure of the longevity of the engineered cell population following expansion and contraction. The duration of the expansion, contraction and persistence phases are evaluated using a pharmacokinetic profile. In some embodiments, a pharmacokinetic (PK) profile is a description of the cells measured in a given tissue over time and is readily ascertained by one skilled in the art by measuring the cells in a given tissue (e.g., peripheral blood) at multiple time points. In some embodiments, a measure of a PK profile provides a method of evaluating or monitoring the effectiveness of the engineered cell therapy in a subject (e.g., having cancer). In some embodiments, a measure of a PK profile provides a method of evaluating the persistence of the engineered cells in a subject. In some embodiments, a PK profile provides a method of evaluating the expansion of the engineered cells in a subject. In some embodiments, a measure of persistence of engineered cells in a subject is used to evaluate the effectiveness of engineered cell therapy in a subject. In some embodiments, a measure of expansion of engineered cells in a subject is used to evaluate the effectiveness of engineered cell therapy in a subject.

A PK profile can be prepared by measuring a quantity of engineered cells in a sample of a given tissue type (e.g., peripheral blood) collected from a recipient and repeating the assessment at different time points. In some embodiments, a baseline tissue sample is collected from a recipient no more than 1 day, 2 days, 3 days, 4 days, 5 days, 6 days, 7 days, 8 days, 9 days, 10 days, 12 days, 13 days, 14 days, or 15 days prior to administration. In some embodiments, tissue collection from a recipient is performed within 0.25-2 hours, within 1-3 hours, within 2-6 hours, within 3-11 hours, within 4-20 hours, or within 5-8 hours of the time of administration of engineered cells. In some embodiments, tissue collection from a recipient is performed on a daily basis starting on day 1, day 2, day 3, or day 4 and continuing through at least day 5, day 6, day 7, day 8, day 9, day 10, day 11, day 12, day 13, day 14, day 15, day 16, day 17, day 18, day 19, or day 20. In some embodiments, tissue collection from a recipient is performed at least 1 time, 2 times, 3 times, 4 times, 5 times, or 6 times per week for up to 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks following administration of cells. In some embodiments, tissue collection from a recipient is performed at least 1 time, 2 times, 3 times, 4 times, 5 times, or 6 times per month for up to 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, or 24 months following administration of cells. In some embodiments, tissue collection from a recipient is performed at least 1 time, 2 times, 3 times, 4 times, 5 times, or 6 times per year for up to 1 year, 2 years, 3 years, 4 years, 5 years, 6 year, 7 years, 8 years, 9 years, or 10 years following administration of cells.

In some embodiments, engineered cell persistence is determined as the duration of time from administration wherein a quantity of engineered cells is present that is at least 0.005-0.05%, 0.01-0.1%, 0.05-0.5%, 0.1-1%, 0.5%-5%, 1-10%, 5%-10%, or 10%-15% (e.g., at least 1%, 5%, 10%, or 15%) of the peak quantity of engineered cells. In some embodiments, a persistence of cells is determined by comparing the quantity of cells measured in a given tissue type (e.g., peripheral blood) to the peak quantity of cells that is measured in the same tissue type. In some embodiments, a persistence of cells is determined by comparing the quantity of cells measured in a given subject (e.g., peripheral blood) to the peak quantity of cells that is measured in the same subject. In some embodiments, a persistence of cells is determined by comparing the quantity of cells measured in a given subject (e.g., peripheral blood) to the peak quantity of cells that is measured in a different subject (i.e., a subject with partial response, a subject with complete response).

In some embodiments, a persistence of engineered cells is present in one or more tissue types (e.g., peripheral blood, organs such as lung) following administration wherein engineered cells are administered on day 1. In some embodiments, a persistence of engineered cells is present in one or more tissue types (e.g., peripheral blood, organs such as lung) up to 1 day, 2 days, 3 days, 4, days, 5 days, 6 days, 7 days, 8 days, 9 days, 10 days, 11 days, 12 days, 13 days, 14 days, 15 days, 16 days, 17 days, 18 days, 19 days, 21 days, 21 days, 22 days, 23 days, 24 days, 25 days, 26 days, 27 days, 28 days, 29 days, 30 days, 31 days, 32 days, 33 days, 34 days, or 35 days following administration wherein engineered cells are administered on day 1. In some embodiments, a persistence of engineered cells is present in one or more tissue types (e.g., peripheral blood, organs such as lung) up to 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 21 months, 22 months, 23 months, or 24 months following administration of engineered cells). In some embodiments, a persistence of engineered cells is measured in one or more tissue types (e.g., peripheral blood) up to 1 year, 2 years, 3 years, 4 years, 5 years, 6 years, 7 years, 8 years, 9 years, and 10 years following administration of engineered cells. In some embodiments, a persistence of engineered cells that is at least 10-25 days, at least 25-50 days, at least 50-100 days, at least 100-364 days, at least one year, at least two years, at least three years, at least four years or at least five years from administration wherein engineered cells are administered on day 1 is indicative of a response in a recipient (e.g., complete response or partial response).

In some embodiments, the engineered cells described herein exhibit improved biodistribution and persistence to target tissues (e.g., lungs) following in vivo administration. For example, at least 50% of the engineered cells of a population of cells (e.g., at least 55%, at least 60%, at least 70%, at least 75%, at least 80%, at least 85%, at least 90%, or at least 95% of the engineered cells of a population) can migrate to lungs upon administration to a subject. In some embodiments, less than 50% of the engineered cells of a population of cells can redistribute (i.e., migrate out of the target tissue) after target distribution. For example, less than 50%, less than 30%, less than 25%, less than 20%, less than 15%, less than 10%, or less than 5% of the engineered cells of a population of cells can migrate out of the target tissue (e.g., lung) after target distribution (e.g., 3 days, 4 days, 5 days, 6 days 7 days, 8 days, 9 days, 10 days, 11 days, 12 days, 13 days, 14 days or longer after target distribution). In some embodiments, the engineered cell described herein has a prolonged survival in one or more tissue type (e.g., lung) up to 7 days, 8 days, 9 days, 10 days, 11 days, 12 days, 13 days, 14 days, 15 days, 16 days, 17 days, 18 days, 19 days, 21 days, 21 days, 22 days, 23 days, 24 days, 25 days, 26 days, 27 days, 28 days, 29 days, 30 days, 31 days, 32 days, 33 days, 34 days, or 35 days following administration. In some embodiments, at least 50% of the engineered cells of a population of cells (e.g., at least 55%, at least 60%, at least 70%, at least 75%, at least 80%, at least 85%, at least 90%, or at least 95% of the engineered cells of a population) can still survive 2 weeks after administration.

### Isolation and Purification of Cells

### Purification

In some embodiments, the population of gene-edited cells (e.g., iPSC, iNK, or NK cells) described herein are activated and/or expanded before or after genome editing. In some embodiments, iPSCs are differentiated after gene-editing. In some embodiments, cells are activated and expanded for about 1 day to about 4 days, about 1 day to about 3 days, about 1 day to about 2 days, about 2 days to about 3 days, about 2 days to about 4 days, about 3 days to about 4 days, or about 1 day, about 2 days, about 3 days, or about 4 days prior to genome editing.

Disclosed herein includes a method for substantially isolating cells that express a detectable level of a surface protein from a population of cells comprising engineered NK cells comprising a combination of any of the gene edits described herein (e.g., IL15/IL15Rα KI and FAS KO).

In some embodiments, the disclosure provides a method for isolating a population of cells engineered to express one or CAR comprising: providing the population of cells wherein the engineered CAR cells comprise a disrupted FAS gene; and isolating the population of cells expressing the one or more CAR (e.g., such that >99% of the population comprises the CAR expressing cells).

In some embodiments, the disclosure provides a population of cells comprising engineered CAR NK cells described herein, wherein less than 0.5% of the cells in the population express a detectable level of FAS. In some embodiments, the disclosure provides a population of cells comprising engineered CAR NK cells described herein, wherein less than 0.1%, less than 0.2%, less than 0.3%, less than 0.4%, less than 0.5%, less than 1%, less than 2%, less than 3%, less than 4%, less than 5% or less than 10% of the cells in the population express a detectable level of FAS.

Removal of a subset of cells from a population can be performed using conventional cell purification methods. Non-limiting examples of cell sorting methods include fluorescence-activated cell sorting, immunomagnetic separation, chromatography, and microfluidic cell sorting. In some embodiments, CAR-expressing cells are removed from a population of cells comprising engineered NK cells by immunomagnetic separation. In some embodiments, IL15/IL15Rα-expressing cells are removed from a population of cells comprising engineered NK cells by immunomagnetic separation.

In some embodiments, genome edited cells are sorted into single cells. In some embodiments, single cell isolates of gene-edited cells are grown into single cell clonal populations. In some embodiments, multiple single-cell clones are generated. In some embodiments, an edited clone is expanded to generate a master cell bank (MCB).

### Formulations and Administrations

### Formulation and Delivery for Gene Editing

Guide RNAs, polynucleotides, e.g., polynucleotides that encode any protein described herein or polynucleotides that encode an endonuclease, and endonucleases as described herein may be formulated and delivered to cells in any manner known in the art.

Guide RNAs and/or polynucleotides may be formulated with pharmaceutically acceptable excipients such as carriers, solvents, stabilizers, adjuvants, diluents, etc., depending upon the particular mode of administration and dosage form. Guide RNAs and/or polynucleotides compositions can be formulated to achieve a physiologically compatible pH, and range from a pH of about 3 to a pH of about 11, about pH 3 to about pH 7, depending on the formulation and route of administration. In some cases, the pH can be adjusted to a range from about pH 5.0 to about pH 8. In some cases, the compositions can comprise a therapeutically effective amount of at least one compound as described herein, together with one or more pharmaceutically acceptable excipients. Optionally, the compositions can comprise a combination of the compounds described herein, or can include a second active ingredient useful in the treatment or prevention of bacterial growth (for example and without limitation, anti-bacterial or anti-microbial agents), or can include a combination of reagents of the present disclosure.

Suitable excipients include, for example, carrier molecules that include large, slowly metabolized macromolecules such as proteins, polysaccharides, polylactic acids, polyglycolic acids, polymeric amino acids, amino acid copolymers, and inactive virus particles. Other exemplary excipients can include antioxidants (for example and without limitation, ascorbic acid), chelating agents (for example and without limitation, EDTA), carbohydrates (for example and without limitation, dextrin, hydroxyalkylcellulose, and hydroxyalkylmethylcellulose), stearic acid, liquids (for example and without limitation, oils, water, saline, glycerol and ethanol), wetting or emulsifying agents, pH buffering substances, and the like.

Guide RNA polynucleotides (RNA or DNA) and/or endonuclease polynucleotide(s) (RNA or DNA) can be delivered by viral or non-viral delivery vehicles known in the art. Alternatively, endonuclease polypeptide(s) can be delivered by viral or non-viral delivery vehicles known in the art, such as electroporation or lipid nanoparticles. In further alternative aspects, the DNA endonuclease can be delivered as one or more polypeptides, either alone or pre-complexed with one or more guide RNAs, or one or more crRNA together with a tracrRNA.

Polynucleotides can be delivered by non-viral delivery vehicles including, but not limited to, nanoparticles, liposomes, ribonucleoproteins, positively charged peptides, small molecule RNA-conjugates, aptamer-RNA chimeras, and RNA-fusion protein complexes. Some exemplary non-viral delivery vehicles are described in Peer and Lieberman, Gene Therapy, 2011, 18: 1127-1133 (which focuses on non-viral delivery vehicles for siRNA that are also useful for delivery of other polynucleotides).

For polynucleotides of the disclosure, the formulation may be selected from any of those taught, for example, in International Application PCT/US2012/069610. Polynucleotides, such as guide RNA, sgRNA, and mRNA encoding an endonuclease, may be delivered to a cell or a subject by a lipid nanoparticle (LNP). An LNP can refer to any particle having a diameter of less than 1000 nm, 500 nm, 250 nm, 200 nm, 150 nm, 100 nm, 75 nm, 50 nm, or 25 nm. Alternatively, a nanoparticle may range in size from 1-1000 nm, 1-500 nm, 1-250 nm, 25-200 nm, 25-100 nm, 35-75 nm, or 25-60 nm. LNPs may be made from cationic, anionic, or neutral lipids. Neutral lipids, such as the fusogenic phospholipid DOPE or the membrane component cholesterol, may be included in LNPs as “helper lipids” to enhance transfection activity and nanoparticle stability. Limitations of cationic lipids include low efficacy owing to poor stability and rapid clearance, as well as the generation of inflammatory or anti-inflammatory responses. LNPs may also be comprised of hydrophobic lipids, hydrophilic lipids, or both hydrophobic and hydrophilic lipids.

Any lipid or combination of lipids known in the art can be used to produce an LNP. Examples of lipids used to produce LNPs include, but are not limited to, DOTMA, DOSPA, DOTAP, DMRIE, DC-cholesterol, DOTAP-cholesterol, GAP-DMORIE-DPyPE, and GL67A-DOPE-DMPE-polyethylene glycol (PEG). Examples of cationic lipids are: 98N12-5, C12-200, DLin-KC2-DMA (KC2), DLin-MC3-DMA (MC3), XTC, MD1, and 7C1. Examples of neutral lipids are: DPSC, DPPC, POPC, DOPE, and SM. Examples of PEG-modified lipids are: PEG-DMG, PEG-CerC14, and PEG-CerC20. The lipids can be combined in any number of molar ratios to produce an LNP. In addition, the polynucleotide(s) can be combined with lipid(s) in a wide range of molar ratios to produce an LNP.

A recombinant adeno-associated virus (rAAV) vector can be used for delivery. Techniques to produce rAAV particles, in which an AAV genome to be packaged that includes the polynucleotide to be delivered, rep and cap genes, and helper virus functions are provided to a cell are standard in the art. Production of rAAV typically requires that the following components are present within a single cell (denoted herein as a packaging cell): an rAAV genome, AAV rep and cap genes separate from (i.e., not in) the rAAV genome, and helper virus functions. The AAV rep and cap genes may be from any AAV serotype for which recombinant virus can be derived, and may be from a different AAV serotype than the rAAV genome ITRs, including, but not limited to, AAV serotypes described herein. Production of pseudotyped rAAV is disclosed in, for example, international patent application publication number WO 01/83692.

### Formulation and Administration of Cells

Genetically modified cells, as described herein may be formulated and administered to a subject by any manner known in the art. Disclosed herein include compositions. In some embodiments, the compositions comprise any population of cells described herein. Also, disclosed herein include methods of obtaining cells for administration to a subject in need thereof, the method comprising: (a) obtaining or having obtained any of the population of cells described herein, and (b) maintaining the population of cells for a time and under conditions sufficient for the one or more engineered cells to differentiate into lineage-restricted progenitor cells or fully differentiated somatic cells. There are provided, methods for treating a subject in need thereof. In some embodiments, the method comprises: (a) obtaining or having obtained any of the population of cells disclosed herein; and (b) administering the lineage-restricted progenitor cells or fully differentiated somatic cells to the subject.

The terms “administering,” “introducing, “implanting,” “engrafting” and “transplanting” are used interchangeably in the context of the placement of cells, e.g., progenitor cells, into a subject, by a method or route that results in at least partial localization of the introduced cells at a desired site. The cells e.g., progenitor cells, or their differentiated progeny can be administered by any appropriate route that results in delivery to a desired location in the subject where at least a portion of the implanted cells or components of the cells remain viable. The period of viability of the cells after administration to a subject can be as short as a few hours, e.g., twenty-four hours, to a few days, to as long as several years, or even the lifetime of the subject, i.e., long-term engraftment. In some embodiments, a genetically modified cell as described herein is viable after administration to a subject for a period that is longer than that of an unmodified cell.

In some embodiments, a composition comprising cells as described herein are administered by a suitable route, which may include intravenous administration, e.g., as a bolus or by continuous infusion over a period of time. In some embodiments, intravenous administration may be performed by intramuscular, intraperitoneal, intracerebrospinal, subcutaneous, intra-articular, intrasynovial, or intrathecal routes. In some embodiments, a composition may be in solid form, aqueous form, or a liquid form. In some embodiments, an aqueous or liquid form may be nebulized or lyophilized. In some embodiments, a nebulized or lyophilized form may be reconstituted with an aqueous or liquid solution.

A cell composition can also be emulsified or presented as a liposome composition, provided that the emulsification procedure does not adversely affect cell viability. The cells and any other active ingredient can be mixed with excipients that are pharmaceutically acceptable and compatible with the active ingredient, and in amounts suitable for use in the therapeutic methods described herein. Additional agents included in a cell composition can include pharmaceutically acceptable salts of the components therein. Pharmaceutically acceptable salts include the acid addition salts (formed with the free amino groups of the polypeptide) that are formed with inorganic acids, such as, for example, hydrochloric or phosphoric acids, or such organic acids as acetic, tartaric, mandelic and the like. Salts formed with the free carboxyl groups can also be derived from inorganic bases, such as, for example, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active compound used in the cell compositions that is effective in the treatment of a particular disorder or condition can depend on the nature of the disorder or condition, and can be determined by standard clinical techniques.

In some embodiments, a composition comprising cells may be administered to a subject, e.g., a human subject, who has, is suspected of having, or is at risk for a disease. In some embodiments, a composition may be administered to a subject who does not have, is not suspected of having or is not at risk for a disease. In some embodiments, a subject is a healthy human. In some embodiments, a subject e.g., a human subject, who has, is suspected of having, or is at risk for a genetically inheritable disease. In some embodiments, the subject is suffering or is at risk of developing symptoms indicative of a disease. In some embodiments, the subject is suffering from, suspected of having, or is at risk for cancer.

### Treatment Methods

The methods, compositions and kits disclosed herein can be used to treat various types of cancer, including but not limited to, melanoma (e.g., metastatic malignant melanoma), renal cancer (e.g., clear cell carcinoma), prostate cancer (e.g., hormone refractory prostate adenocarcinoma), pancreatic adenocarcinoma, breast cancer, colon cancer, lung cancer (e.g., non-small cell lung cancer (NSCLC) and small-cell lung cancer (SCLC)), esophageal cancer, squamous cell carcinoma of the head and neck, liver cancer, ovarian cancer, cervical cancer, thyroid cancer, glioblastoma, glioma, leukemia, lymphoma, and other neoplastic malignancies. Additionally, the disease or condition provided herein includes refractory or recurrent malignancies whose growth may be inhibited using the methods and compositions disclosed herein. In some embodiments, the cancer is carcinoma, squamous carcinoma, adenocarcinoma, sarcomata, endometrial cancer, breast cancer, ovarian cancer, cervical cancer, fallopian tube cancer, primary peritoneal cancer, colon cancer, colorectal cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, glioblastoma, glioma, squamous cell carcinoma of the head and neck, prostate cancer, pancreatic cancer, mesothelioma, sarcoma, hematological cancer, leukemia, lymphoma, neuroma, or a combination thereof. In some embodiments, the cancer is carcinoma, squamous carcinoma (e.g., cervical canal, eyelid, tunica conjunctiva, vagina, lung, oral cavity, skin, urinary bladder, tongue, larynx, and gullet), and adenocarcinoma (e.g., prostate, small intestine, endometrium, cervical canal, large intestine, lung, pancreas, gullet, rectum, uterus, stomach, mammary gland, and ovary). In some embodiments, the cancer is sarcomata (e.g., myogenic sarcoma), leukosis, neuroma, melanoma, and lymphoma.

Provided herein, in some embodiments, are methods for treating cancer using any engineered cells described herein (or any population of cells described herein). In some embodiments, the cancer is lung cancer. The lung cancer can be non-small cell lung cancer or small cell lung cancer.

In some embodiments, a method for treating a subject in need thereof comprises (a) obtaining or having obtained the population of cells described herein or obtaining or having obtained the population of cells described herein following differentiation into lineage-restricted progenitor cells or fully differentiated somatic cells and (b) administering the lineage-restricted progenitor cells or fully differentiated somatic cells to the subject. The lineage-restricted progenitor cells can be hematopoietic progenitor cells, mesodermal cells, definitive hemogenic endothelium, definitive hematopoietic stem or progenitor cells, CD34+ cells, multipotent progenitors (MPP), common lymphoid progenitor cells, T cell progenitors, NK cell progenitors, pancreatic endoderm progenitors, pancreatic endocrine progenitors, mesenchymal progenitor cells, muscle progenitor cells, blast cells, or neural progenitor cells, and the fully differentiated somatic cells can be pancreatic beta cells, epithelial cells, endodermal cells, macrophages, hepatocytes, adipocytes, kidney cells, blood cells, cardiomyocytes, or immune system cells.

In some embodiments, the methods comprise delivering the engineered cells of the present disclosure to a subject having a cancer (e.g., lung cancer), wherein the cells express an IL15/IL15Rα fusion protein and a CAR and have disrupted expressions of FAS. The CAR can be an anti-GPR87 CAR. In some embodiments, the engineered cells described herein demonstrate enhanced in vivo efficacy in treating lung cancer because of their co-localization with lung cancer cells.

The step of administering can include the placement (e.g., transplantation) of cells, e.g., engineered NK cells, into a subject, by a method or route that results in at least partial localization of the introduced cells at a desired site, such as tumor, such that a desired effect(s) is produced. Engineered cells can be administered by any appropriate route that results in delivery to a desired location in the subject where at least a portion of the implanted cells or components of the cells remain viable. The period of viability of the cells after administration to a subject can be as short as a few hours, e.g., twenty-four hours, to a few days, to as long as several years, or even the life-time of the subject, i.e., long-term engraftment. For example, in some embodiments, an effective amount of engineered NK cell is administered via a systemic route of administration, such as an intraperitoneal or intravenous route.

The genetically modified cells described herein or cells derived/differentiated from the genetically modified cells described here can be administered to the subject in need thereof one or more times, for example once, twice, three times, four times, five times, or six times. It can be advantageous, in some embodiments, to provide a single administration of the cells to the subject. In some embodiments, it can be advantageous to provide up to three administrations (e.g., one, two or three administrations) of the cells to the subject. Any of the two administrations can be, for example, one day to one year part. For example, the first administration can be, or be about, 1 to 21 days apart (e.g., one day, two days, three days, four days, five days, six days, seven days, ten days, two weeks, three weeks, or a value or a range between any two of these values) apart from the second administration. As another example, the second administration can be, or be about, 1 day to one year (e.g., one day, two days, three days, four days, five days, six days, seven days, two weeks, three weeks, four weeks, five weeks, six weeks, two months, three months, six months, a year, or a value or a range between any two of these values) apart from the third administration. When there are three or more administrations, the interval between any of the two adjacent administrations can be the same or different in length. For example, in some embodiments, the first administration is about a week (e.g., 7 days) apart from the second administration, and the second administration is about five weeks (e.g., 35 days) apart from the third administration. The method described herein, in some embodiments, does not comprise regular on-schedule administration of the cells, e.g., weekly, biweekly, monthly, bimonthly, quarterly, biquarterly, yearly, or biyearly administration. In some embodiment, the method described herein does not comprise any administration of the cells three months, six months, nine months, a year, two years, or longer, after the first, second, or third administration of the cells. In some embodiment, the method described herein does not comprise any administration of the cells after the second or third administration of the cells.

A subject may be any subject for whom diagnosis, treatment, or therapy is desired. In some embodiments, the subject is a mammal. In some embodiments, the subject is a human. In some embodiments, an engineered NK cell population being administered according to the methods described herein comprises gene edited hematopoietic cells (e.g., NK cells) differentiated from gene-edited stem cells (e.g., iPSC cells). In some embodiments, an engineered cell population (e.g., comprising NK cells) being administered according to the methods described herein does not induce toxicity in the subject, e.g., the engineered NK cells do not induce toxicity in non-cancer cells. In some embodiments, an engineered cell population (e.g., NK cells) being administered does not trigger complement mediated lysis, or does not stimulate antibody-dependent cell mediated cytotoxicity (ADCC). In some embodiments, the subject being treated has no chronic immune suppression.

An effective amount refers to the amount of a population of engineered cells (e.g., NK cells) needed to prevent or alleviate at least one or more signs or symptoms of a medical condition (e.g., cancer), and relates to a sufficient amount of a composition to provide the desired effect, e.g., to treat a subject having a medical condition. An effective amount also includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

In some embodiments, a subject is administered a population of cells comprising any of the engineered NK cells disclosed herein. In some embodiments, a subject is administered a population of cells comprising any of the engineered NK cells disclosed herein at a dose in the range of about 1×10⁷ to 3×10⁸ engineered cells. In some embodiments, a subject is administered a population of cells comprising any of the engineered cells disclosed herein at a dose in the range of about 3×10⁷ to 3×10⁸ engineered cells. In some embodiments, the cells are derived from iPSCs. In some embodiments, the cells are expanded in culture prior to administration to a subject in need thereof.

Modes of administration include but are not limited to injection and infusion. In some embodiments, injection includes, without limitation, intravenous, intrathecal, intraperitoneal, intraspinal, intracerebrospinal, and intrasternal infusion. In some embodiments, the route is intravenous. In some embodiments, cells are administered as a bolus or by continuous infusion (e.g., intravenous infusion) over a period of time. In some embodiments, cells described herein are administered in several doses over a period of time (e.g., several infusions over a period of time). The cells described herein can be administered in a single dose or in 2, 3, 4, 5, 6 or more doses (or infusions). In some embodiments, the subject being treated is dosed (e.g., with an infusion) about every 1, 2, 3, 4, 5, 6, 7 or 8 weeks. In some embodiments, the subject being treated is dosed (e.g., with an infusion) every 2-4 weeks (e.g., every 2 weeks, 3 weeks or 4 weeks). In some embodiments, engineered cells (e.g., NK cells) are administered systemically, which refers to the administration of a population of cells other than directly into a target site, tissue, or organ, such that it enters, instead, the subject's circulatory system and, thus, is subject to metabolism and other like processes.

The efficacy of a treatment comprising a composition for the treatment of a medical condition can be determined by the skilled clinician. A treatment is considered “effective treatment,” if any one or all of the signs or symptoms of, as but one example, levels of functional target are altered in a beneficial manner (e.g., increased by at least 10%), or other clinically accepted symptoms or markers of disease (e.g., cancer) are improved or ameliorated. Efficacy can also be measured by failure of a subject to worsen as assessed by hospitalization or need for medical interventions (e.g., progression of the disease is halted or at least slowed). Methods of measuring these indicators are known to those of skill in the art and/or described herein. Treatment includes any treatment of a disease in subject and includes: (1) inhibiting the disease, e.g., arresting, or slowing the progression of symptoms; or (2) relieving the disease, e.g., causing regression of symptoms; and (3) preventing or reducing the likelihood of the development of symptoms.

The dosage of the NK cells of the disclosure administered to a subject (e.g., a subject suffering from cancer) can vary. The method can comprise administering at most, at least, or about 1×10² to 1×10¹⁰ per gram (e.g., per gram of body weight) to the subject (e.g., 100, 1000, 1×10⁴, 1×10⁵, 1×10⁶, 1×10⁷, 1×10⁸, 1×10⁹, 1×10¹⁰ cells per gram or a number or a range between any two of these values). The method can comprise administering about 1×10⁶ NK cells per gram to the subject.

The NK cells can be administered to the subject once. The NK cells can be administered to the subject more than once. The NK cells can be administered to the subject at least three times. The NK cells can be administered to the subject in a cycle of at least 7 days. The NK cells can be administered to the subject one, two, or three times in a week. In some embodiments, the NK cells are administered to the subject three times in seven to eight days.

The treatment of the present disclosure can comprise administration of NK cells obtained by the disclosed method (e.g., that are engineered to express anti-GPR87 CAR) for a desired duration in a cycle. The administration of NK cells can be daily or with break(s) between days of administrations. The break can be, for example, 1 day, 2 days, 3 days, 4 days, 5 days, 6 days, 7 days, 8 days, 9 days, 10 days, 11 days, 12 days, 13 days, 14 days, or more. The administration can be, for example, once every two days, every three days, every four days, every five days, every six days, or every seven days. The length of the desired duration can vary, for example, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, or more days. Each cycle of treatment can have various lengths, for example, at least 14 days, 15 days, 16 days, 17 days, 18 days, 19 days, 20 days, 21 days, 22 days, 23 days, 24 days, 25 days, 26 days, 27 days, 28 days, or more. For example, a single cycle of the treatment can comprise administration of the NK cells for four days, five days, six days, seven days, eight days, nine days, ten days, eleven days, twelve days, thirteen days, fourteen days, fifteen days, sixteen days, seventeen days, eighteen days, nineteen days, twenty days, twenty-one days, twenty-two days, twenty-three days, twenty-four days, twenty-five days, twenty-six days, twenty-seven days, twenty-eight days, or more in a cycle (e.g., in a cycle of at least 21 days (e.g., 21 to 28 days)). In some embodiments, the treatment can comprise administration of NK cells for, or for at least, four days, five days, six days, seven days, eight days, nine days, ten days, or a range between any two of these values, in a cycle (e.g., a cycle of at least 21 days (e.g., 21 to 28 days)). The administration of NK cells in a single cycle of the treatment can be continuous or with one or more intervals (e.g., one day or two days of break). In some embodiments, the treatment comprises administration of the NK cells for three days in a cycle of 7 to 8 days. Depending on the needs of inhibition/reversion of cancer progression in the subject, the subject can receive one, two, three, four, five, six, or more cycles of treatment.

Inhibiting progression of the cancer can comprise inhibition of growth of one or more tumors in the subject and/or reducing the number of cancer cells detected in the subject; relative to an untreated subject. Inhibiting progression of the cancer can comprise inhibition of growth of one or more tumors in the subject and/or reducing the number of cancer cells detected in the subject; relative to the subject prior to administration of the NK cells. In some embodiments, the number of cancer cells detected in the subject increases by no more than 0.5-fold after administration of the NK cells, following one or more cycles of treatment. In some embodiments, the number of cancer cells detected in the subject does not significantly increase following one or more cycles of treatment. In some embodiments, the number of cancer cells detected in the subject decreases following one or more cycles of treatment. In some embodiments, the growth of at least one of the one or more tumors in the subject is inhibited by at least about 70%, about 75%, about 80%, about 85%, about 90%, about 95%, or about 100%, following one or more cycles of treatment. In some embodiments, the subject is tumor-free following one or more cycles of treatment. Methods for determining effectiveness of treatment (e.g., inhibition of the progression of the cancer) are known by persons skilled in the art, and are also described below.

### Methods for Determining Treatment Efficacy and Status for Cancer

Also disclosed herein include methods for determining clinical outcome for a treatment of cancer of the present disclosure, monitoring of the treatment, determining responsiveness of a subject to the treatment, determining the status of the cancer in a subject, and improving treatment outcome. The methods can be used to guide the treatment, provide treatment recommendations, and/or reduce or avoid unnecessary ineffective treatment for patients.

In some embodiments, determining clinical outcome for a treatment of cancer of the present disclosure, monitoring of the treatment, determining responsiveness of a subject to the treatment, and/or determining the status of the cancer in a subject comprises determining approximate cancer cell number, and/or measurement of tumor size and/or inhibition of tumor growth. Inhibiting progression of the cancer can comprise inhibition of growth of one or more tumors in the subject. Inhibiting progression of the cancer can comprise reducing the number of cancer cells detected in the subject. The term “inhibition of tumor growth” can refer to causing a reduction in or complete cessation of tumor growth and/or causing a regression in tumor size (e.g., volume). The term “tumor volume” or “tumor size” can refer to the total size of the tumor, which can include the tumor itself plus affected lymph nodes if applicable. Tumor size can be determined by a variety of methods known in the art, such as, e.g., by measuring the dimensions of the tumor using calipers, computed tomography (CT) or magnetic resonance imaging (MRI) scans, mammography, and X-ray; and calculating the volume using equations based on, for example, the z-axis diameter, or on standard shapes such as the sphere, ellipsoid, or cube. Tumor size may be assessed at any time before, during or following at least one cycle of treatment with engineered NK cells. Tumor size can be assessed at a first timepoint, and at one or more additional timepoints. In some embodiments, tumor size can be assessed in the subject and, e.g., an untreated subject at equivalent timepoints (e.g., at a first timepoint, and at one or more additional timepoints). Tumor growth can be determined by, e.g., measuring tumor size at a first timepoint and measuring tumor size at one or more additional timepoints. In some embodiments, increased inhibition of tumor growth in the subject indicates the subject as responsive to the cancer treatment.

The inhibition of growth of at least one of the one or more tumors in the subject can be increased by, by about, by at least, or by at least about 25%, 30%, 35%, 40%, 50%, 55%, 60%, 65%, 70%, 75%, 80%, 85%, 90%, 95%, 100% or a number or a range between any two of these values, relative to an untreated subject or the subject prior to administration of the NK cells, following one or more cycles of treatment. The growth of at least one of the one or more tumors in the subject can be inhibited by, by about, by at least, or by at least about 50%, 55%, 60%, 65%, 70%, 75%, 80%, 85%, 90%, 95%, 100% or a number or a range between any two of these values relative to an untreated subject or the subject prior to administration of the NK cells, following one or more cycles of treatment. The growth of at least one of the one or more tumors in the subject can be inhibited by, by about, by at least, or by at least about 70%, 75%, 80%, 85%, 90%, 95%, 100% or a number or a range between any two of these values relative to an untreated subject or the subject prior to administration of the NK cells, following one or more cycles of treatment. The subject can be tumor-free following one or more cycles of treatment.

In some embodiments, the number of cancer cells detected in the subject increases by no more than 0.5-fold (e.g., increases by 0.5-fold, 0.25-fold, 0.1-fold or less) after administration of the NK cells, following one or more cycles of treatment. In some embodiments, the number of cancer cells detected in the subject decreases by 50% or more in the subject (e.g., by 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, or a number or a range between any two of these values), relative to an untreated subject or to the subject prior to administration of the NK cells. In some embodiments, the number of cancer cells detected in the subject does not significantly increase following one or more cycles of treatment. In some embodiments, the number of cancer cells detected in the subject decreases following one or more cycles of treatment.

In some embodiments, the method comprises measuring expression of one or more markers in the subject (e.g., in a sample obtained from the subject). The method can comprise: measuring expression of one or more markers for apoptosis, DNA damage, cell cycle or any combination thereof in the subject before and/or after the subject is administered the NK cells. The one or more markers can comprise cleaved-caspase3, γ-H2AX, phosphorylated CHK1, phosphorylated CHK2, or any combination thereof.

A method of determining responsiveness of a subject to a treatment comprising NK cells of the disclosure (e.g., that are engineered to express an anti-GPR87 CAR) can comprise, for example, analyzing a sample (e.g., a biopsy sample) obtained from a subject with cancer, wherein the subject is undergoing a treatment and/or has received the treatment, thereby determining the responsiveness of the subject to the treatment. Samples can be obtained by any method known in the art. In some embodiments, the biological sample is obtained from an animal subject, such as a human subject. A biological sample is any solid or fluid sample obtained from, excreted by or secreted by any living organism, (including samples from a healthy or apparently healthy human subject or a human patient affected by cancer). A biological sample can be a biological fluid obtained from, for example, blood, plasma, serum, urine, bile, ascites, saliva, cerebrospinal fluid, aqueous or vitreous humor, or any bodily secretion, a transudate, an exudate (for example, fluid obtained from an abscess or any other site of infection or inflammation), or fluid obtained from a joint (for example, a normal joint or a joint affected by disease, such as a rheumatoid arthritis, osteoarthritis, gout or septic arthritis). A sample can also be obtained from any organ or tissue (including a biopsy, such as a tumor biopsy) or can include a cell (whether a primary cell or cultured cell) or medium conditioned by any cell, tissue or organ. Exemplary samples include, without limitation, cells, cell lysates, blood smears, cytocentrifuge preparations, cytology smears, bodily fluids (e.g., blood, plasma, serum, saliva, sputum, urine, bronchoalveolar lavage, semen, etc.), tissue biopsies (e.g., tumor biopsies), fine-needle aspirates, and/or tissue sections (e.g., cryostat tissue sections and/or paraffin-embedded tissue sections). The sample can also include circulating tumor cells (which can be identified by cell surface markers). In some embodiments, samples are used directly (e.g., fresh or frozen), or can be manipulated prior to use, for example, by fixation (e.g., using formalin) and/or embedding in wax (such as formalin-fixed paraffin-embedded (FFPE) tissue samples). It can be appreciated that any method of obtaining tissue from a subject can be utilized, and that the selection of the method used will depend upon various factors such as the type of tissue, age of the subject, or procedures available to the practitioner.

The levels of the one or more markers in the subject (e.g., in a biopsy sample obtained from the subject) can be measured by any method known in the art. In some embodiments, the one or more markers are detected by immunofluorescence, mass cytometry (CyTOF), FACS, drop-seq, RNA-seq, single cell qPCR, MERFISH (multiplex (in situ) RNA FISH), microarray and/or by in situ hybridization. Other methods including absorbance assays and colorimetric assays are known in the art and may be used herein. In some aspects, measuring expression of the one or more markers comprises measuring protein expression levels. Protein expression levels may be measured, for example, by performing a Western blot, an ELISA, immunohistochemistry or binding to an antibody array. In another aspect, measuring expression of the one or more markers comprises measuring RNA expression levels. RNA expression levels may be measured by performing RT-PCR, Northern blot, an array hybridization, or RNA sequencing methods.

An enzyme-linked immunosorbent assay, or ELISA, may be used to measure the differential expression of a plurality of markers. There are many variations of an ELISA assay. All are based on the immobilization of an antigen or antibody on a solid surface, generally a microtiter plate. The original ELISA method comprises preparing a sample containing the biomarker proteins of interest, coating the wells of a microtiter plate with the sample, incubating each well with a primary antibody that recognizes a specific antigen, washing away the unbound antibody, and then detecting the antibody-antigen complexes. The antibody-antibody complexes may be detected directly. For this, the primary antibodies are conjugated to a detection system, such as an enzyme that produces a detectable product. The antibody-antibody complexes may be detected indirectly. For this, the primary antibody is detected by a secondary antibody that is conjugated to a detection system, as described above. The microtiter plate is then scanned and the raw intensity data may be converted into expression values using means known in the art.

Detection of the one or more markers can be by FACS. The term “fluorescent activated cell sorting” or “FACS”, as used herein, refers to a technique for counting, examining, and sorting microscopic particles suspended in a stream of fluid. It allows simultaneous multiparametric analysis of the physical and/or chemical characteristics of single cells flowing through an optical and/or electronic detection apparatus. Generally, a beam of light (usually laser light) of a single wavelength is directed onto a hydro-dynamically focused stream of fluid. A number of detectors are aimed at the point where the stream passes through the light beam; one in line with the light beam (Forward Scatter, correlates to cell volume) and several perpendicular to the beam, (Side Scatter, correlates to the inner complexity of the particle and/or surface roughness) and one or more fluorescent detectors. Each suspended particle passing through the beam scatters the light in some way, and fluorescent chemicals found in the particle or attached to the particle may be excited into emitting light at a lower frequency than the light source. By analyzing the combinations of scattered and fluorescent light picked up by the detectors it is then possible to derive information about the physical and chemical structure of each individual particle.

Detection of the one or more markers may involve a cell sorting step to enrich for cells of interest and thus facilitate or enhance their sensitive and specific detection. Cell sorting techniques are commonly based on tagging the cell with antibody against the cell membrane antigen specific to the target subpopulation of cells. The antibody is conjugated to a magnetic bead and/or fluorophore or other label to enable cell sorting and detection. Such methods may include affinity chromatography, particle magnetic separation, centrifugation, or filtration, and flow cytometry (including fluorescence activated cell sorting; FACS).

RNA can be isolated from the cancer cells and can be sequenced by any method known in the art for determining expression of one or more markers. Methods of preparing cDNA are known in the art. Single cells may be sequenced for detection of at least one of the one or more markers. Single cells can be, for example, divided into single droplets using a microfluidic device. The single cells in such droplets may be further labeled with a barcode.

A method of determining responsiveness of a subject to a treatment comprising engineered NK cells of the disclosure can comprise, for example, analyzing circulating tumor DNA (ctDNA) of a subject with cancer, the subject is undergoing a treatment and/or has received the treatment, thereby determining the responsiveness of the subject to the treatment. For example, analyzing ctDNA can comprise detecting variant allele frequency in the ctDNA in a first sample obtained from the subject at a first time point, detecting variant allele frequency in the ctDNA obtained from the subject at one or more additional time points in one or more additional samples, and determining the difference of the variant allele frequency in ctDNA between the first and at least one of the one or more additional samples, a decrease in the variant allele frequency in at least one of the additional samples relative to the first sample indicates the subject as responsive to the cancer treatment.

### Persistence of the Engineered Cells In Vivo

The engineered cells of the disclosure comprise one or more deletions and/or insertions that can, in some embodiments, advantageously contribute to the persistence (e.g., survival) of the engineered cells in a subject. The NK cells can persist in the subject for at least one week following administration. In some embodiments, the NK cells can persist in the subject for at least two weeks, three weeks, or longer following administration.

In some embodiments, the number of NK cells detected in the subject decreases by less than 20% (e.g., 0.000000001%, 0.00000001%, 0.0000001%, 0.000001%, 0.00001%, 0.0001%, 0.001%, 0.01%, 0.1%, 1%, 2%, 3%, 4%, 5%, 6%, 7%, 8%, 9%, 10%, 11%, 12%, 13%, 14%, 15%, 16%, 17%, 18%, 19%, or 20%, or a number or a range between any two of these values) one week after administration. In some embodiments, the number of NK cells detected in the subject decreases by less than 50% (e.g., 0.000000001%, 0.00000001%, 0.0000001%, 0.000001%, 0.00001%, 0.0001%, 0.001%, 0.01%, 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or a number or a range between any two of these values) two weeks after administration. The NK cells can be localized to the site of the cancer following administration. For example, the NK cells described herein can be localized to the lung following administration (see, for example, Examples 1 and 2). In some embodiments, the NK cells migrate to the lung within 5 mins, 10 mins, 20 mins, or 30 mins following administration (e.g., intravenous injection).

### Lymphodepletion Conditioning Therapy

In some embodiments, any engineered cells described herein (or any population of cells described herein) are administered to a subject (e.g., a human patient having a cancer, e.g., a non-Hodgkin lymphoma) after a subject has received a lymphodepleting regimen.

In some embodiments, the lymphodepleting regimen comprises administering at least one chemotherapeutic agent (e.g., cyclophosphamide). In some embodiments, the lymphodepleting regimen comprises administering at least two chemotherapeutic agents (e.g., cyclophosphamide and fludarabine). In some embodiments, the first dose (e.g., infusion) of the engineered cells described herein is administered to a subject after lymphodepletion.

### EXAMPLES

Some aspects of the embodiments discussed above are disclosed in further detail in the following examples, which are not in any way intended to limit the scope of the present disclosure.

### Example 1

### Generation of iNK Cells with IL15/IL15Rα Knock-In, FAS Knock-Out and/or CISH Knock-Out

In this example, exemplary iNK cells comprising different gene edits (e.g., IL15/IL15Rα knock-in, FAS knock-out, and/or CISH knock-out) were generated according to the protocols for the generation of engineered iNK cells with different gene knock-in or knock-out described in, for example, WO 2023/233342 and WO 2022/113056, the contents of which are incorporated herein by reference in their entirety.

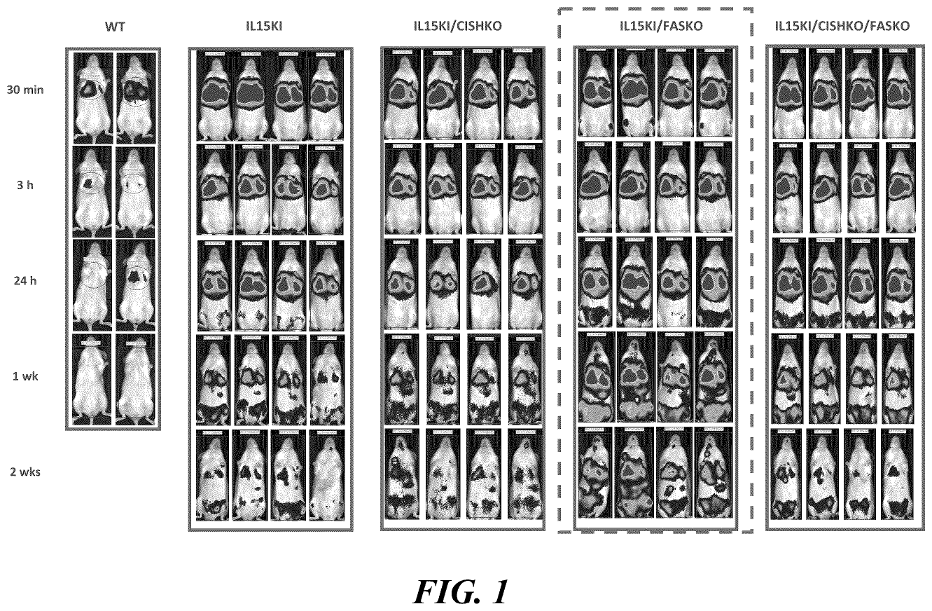

FIG. 1 shows cell viabilities and biodistribution fluorescence imaging of different engineered iNK cells in comparison to WT NK cells. The images demonstrate that majority of the iNK cells migrated to the lungs within 30 min. after IV injection. Most of WT NK cells did not survive (<10% at 3 hr post-injection) while IL15/IL15Rα KI shows some benefits to iNK cell survival (˜35% after 24 hr). IL15/IL15Rα KI/FAS KO significantly improved the survival and distribution of iNK cells (˜80% at 1 wk and 45% at 2 wks).

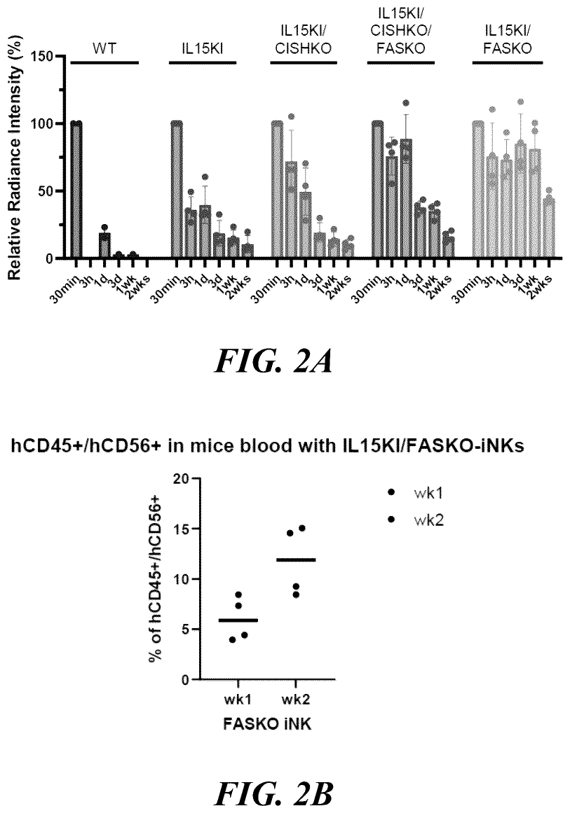

FIG. 2A is a graph showing relative radiance intensity percent of different engineered iNK cells in comparison to WT NK cells. FIG. 2B is a graph showing the hCD45+/hCD56+ percentage in mice blood with IL15/IL15Rα KI/FAS KO-iNKs. Among FAS KO, CISH KO/FAS KO and CISH KO, FAS KO shows the greatest in vivo persistence, followed by CISH KO/FAS KO edits. CISH KO shows the lowest in vivo persistence. The iNK cells with IL15/IL15Rα KI/FAS KO could be detected in the blood (but not for cells without IL15/IL15Rα KI/FAS KO edits).

### Example 2

### In vivo therapeutic study of GPR87 CAR iNKs in treating lung cancer

In this example, GPR87 CAR iNKs with various gene edits (e.g., IL15/IL15Rα KI, CISH KO, and FAS KO) were generated and evaluated for their in vivo therapeutic efficacy in treating lung cancer. It is expected that the in vivo co-localization of iNK cells and lung cancer cells can favor the therapeutic efficacy against lung cancer.

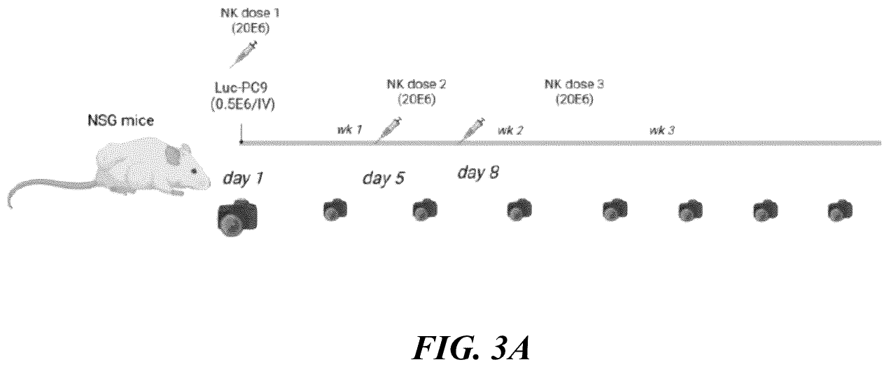

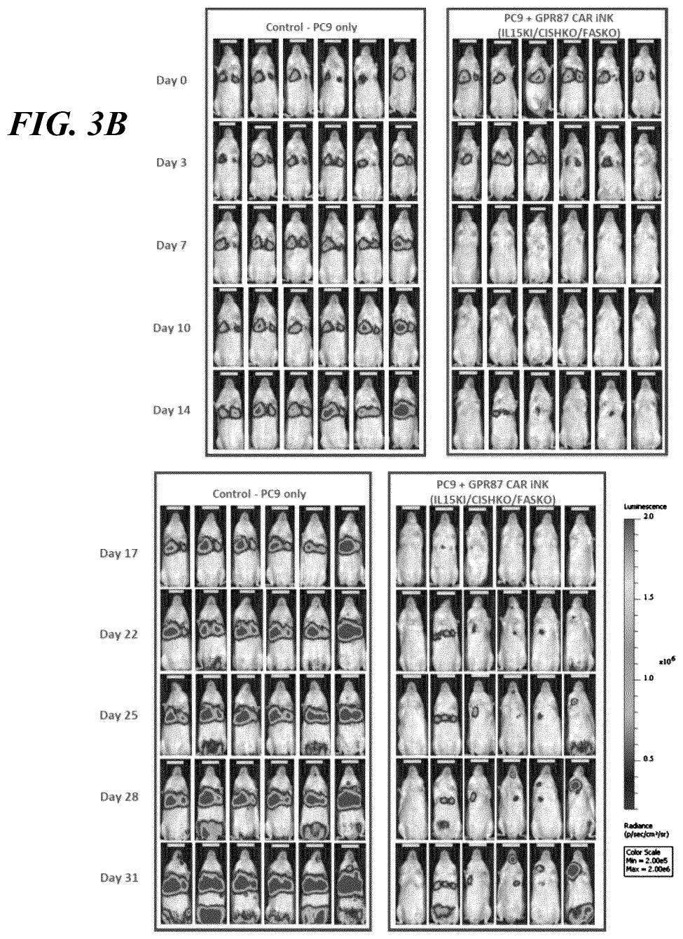

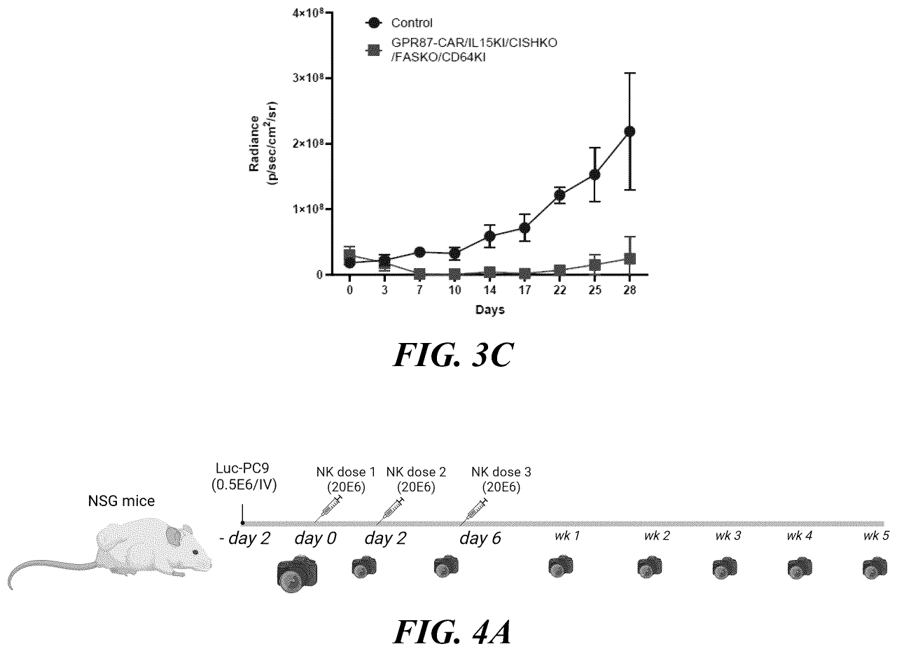

In a first in vivo therapeutic study, mice were intravenously injected with 0.5×10⁶ PC-9 cancer cells (high GPR87 expressing cells) labeled with luciferase and 20×10⁶ iNK cells comprising anti-GPR87 CAR, IL15/IL15-α KI, CISH KO, and FAS KO. Two more intravenous injections of 20×10⁶ iNK cells were given at days 5 and 8. The mice were imaged for cancer cell localization from day 0 to day 31. FIG. 3A presents a schematic for an in vivo protocol to test the therapeutic efficacy of iNK cells comprising anti-GPR87 CAR, IL15/IL15Rα KI, CISH KO, and FAS KO. FIG. 3B shows cell viabilities and biodistribution fluorescence imaging of engineered iNK cells comprising anti-GPR87 CAR, IL15/IL15Rα KI, CISH KO, and FAS KO in comparison to control cells. FIG. 3C is a plot showing the radiance intensity of the engineered iNK cells in comparison to the control cells.

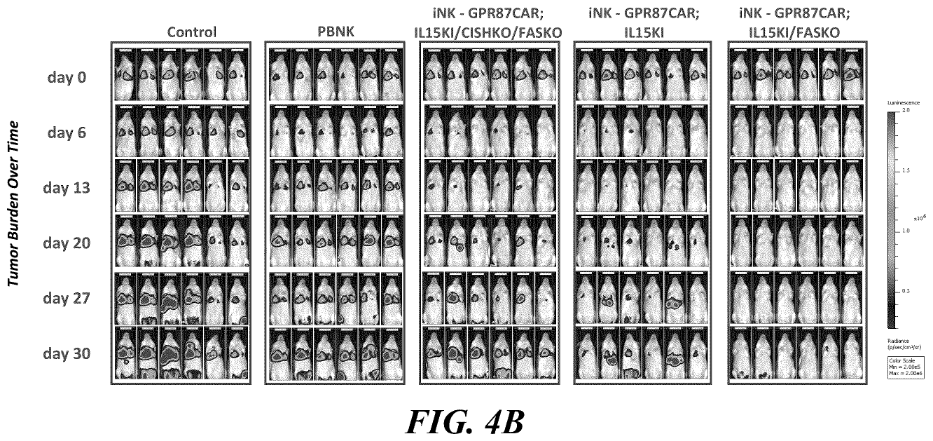

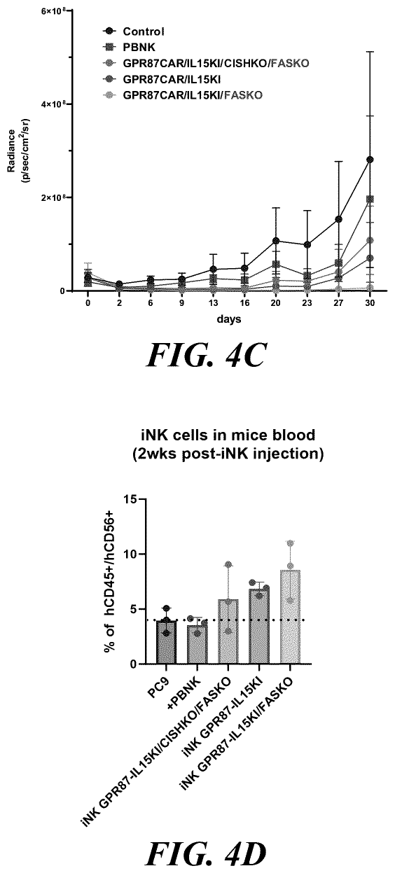

In a second in vivo therapeutic study, mice were intravenously injected with 0.5×10⁶ PC9 cancer cells (high GPR87 expressing cells) labeled with luciferase. Two days later (day 0), 20×10⁶ iNK cells were intravenously injected into the mice. Three different iNK cells were evaluated: iNK cells comprising anti-GPR87 CAR, IL15/IL15Rα KI, CISH KO, and FAS KO, iNK cells comprising anti-GPR87 CAR and IL15/IL15Rα KI, and iNK cells comprising anti-GPR87 CAR, IL15/IL15Rα KI, and FAS KO. Two more intravenous injections of 20×10⁶ iNK cells were given at days 2 and 6. The mice were imaged for cancer cell localization from day 0 to day 30. FIG. 4A presents a schematic of this protocol.

FIG. 4B shows cell viabilities and biodistribution fluorescence imaging of different engineered iNK cells in comparison to control cells. FIG. 4C is a plot showing the radiance of different engineered iNK cells. FIG. 4D is a plot showing the percentage of hCD45+/hCD56+ cells in mice blood 2 wks post-iNK injection. Compared to peripheral blood NK cells, anti-GPR87 CAR iNK cells comprising IL15/IL15Rα KI/FAS KO demonstrated the highest in vivo killing efficacy, while GPR87 CAR iNK cells comprising IL15/IL15Rα KI/FAS KO/CISH KO demonstrated the lowest in vivo killing efficacy. The GPR87 CAR iNK cells comprising IL15/IL15Rα KI and FAS KO could eliminate the PC-9 cancer cells after 2 doses and did not show relapse until day 27. The efficacy data is also consistent with the biodistribution data.

Taken together, in comparison to iNK cells that have a relatively short in vivo half life, IL15 KI can benefit to the iNK survival while IL15/IL15Rα KI and FAS KO together can significantly prolong the in vivo iNK survival up to 2 weeks by 50%. It was also observed that majority of iNK cells quickly migrated to the lungs, while only a small part of the remaining iNKs can redistribute (migrate out of the lungs) between 3 days to 1-week post-injection. The in vivo data suggests that as expected GPR87 CAR iNK cells showed good in vivo efficacy due to their co-localization with lung cancer cells. In the treatment of tumors that are not located in the lungs (e.g., liver cancer or Hodgkin's lymphoma), gene editing to enhance the iNK redistribution is needed to achieve better therapeutic efficacy.

### Example 3

### New Edits Screening in iNK Cells

Various gene edits and modifications were evaluated in this example to enable a differentiated iNK product with efficient in vitro differentiation and increased cytotoxicity as well as enhanced in vivo persistence (e.g., in bone marrow/blood), biodistribution and efficacy.

iNK cells comprising the edits of B2M KO/SERPINB9-P2A-IL15/IL15Rα KI, CIITA KO/CD30 CAR-P2A-HLA-E trimer KI, CISH KO and FAS KO (that is, the iNK cells having IL15/IL15Rα fusion and SERPINB9 linked with a P2A knocked into B2M KO, a CD30 CAR and HLA-E trimer linked with a P2A knocked into CIITA KO, a CISH KO and a FAS KO) were treated with different interleukins (IL2, IL12, and/or IL18). The cytotoxicity of interleukin-treated iNK cells was determined using a killing assay against L540 Hodgkin lymphoma.

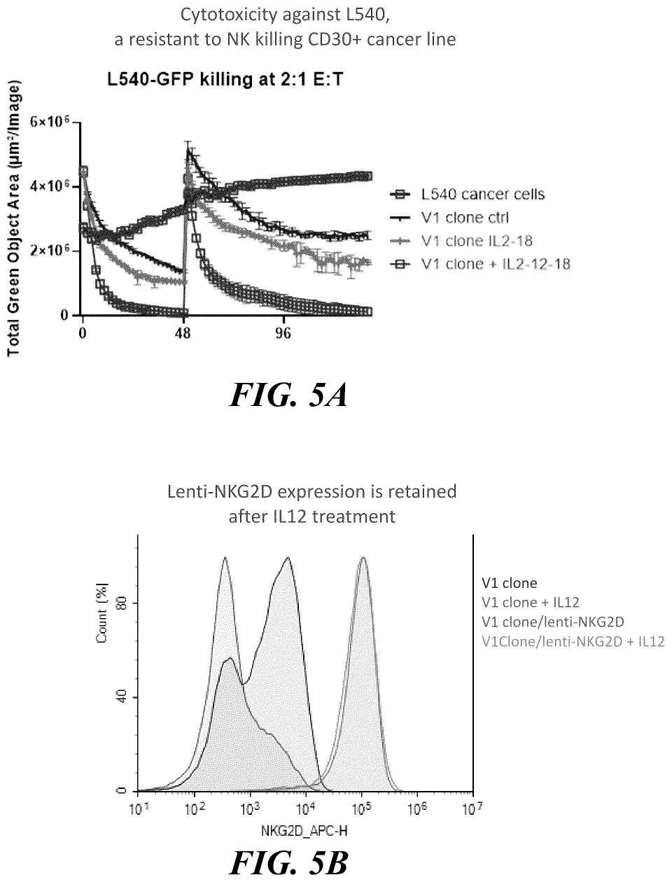

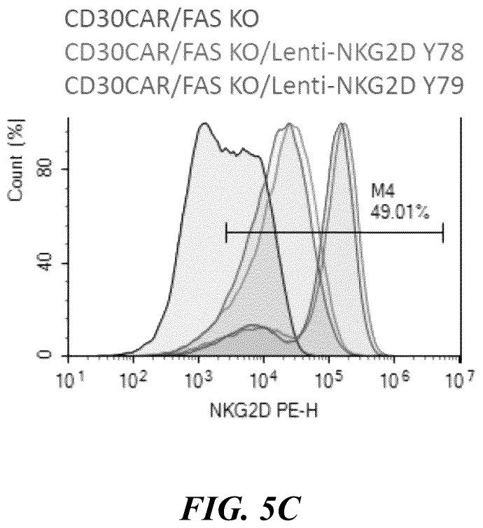

FIG. 5A provides a graph demonstrating L540 cell killing by iNK cells treated with interleukins IL2/IL18 and IL2/IL12/IL18 in comparison to control iNK cells. Effector and L540 cells were plated at 2:1 effector:target (E:T) ratio. FIG. 5B is a graph showing the NKG2D expression level of CD30+ iNK cells overexpressing NKG2D with or without IL12 treatment. The data indicates that Lenti-viral NKG2D expression was retained after IL12 treatment and that iNK treatment with IL12 improves in vitro cytotoxicity. FIG. 5C is a graph showing the NKG2D expression level of CD30+ iNK cells comprising FAS knock-out. Both NKG2D-Y79 construct and NKG2D-Y78 lenti-viral constructs were expressed in over 95% cells. NKG2D-Y79 construct was expressed at a higher level than NKG2D-Y78.

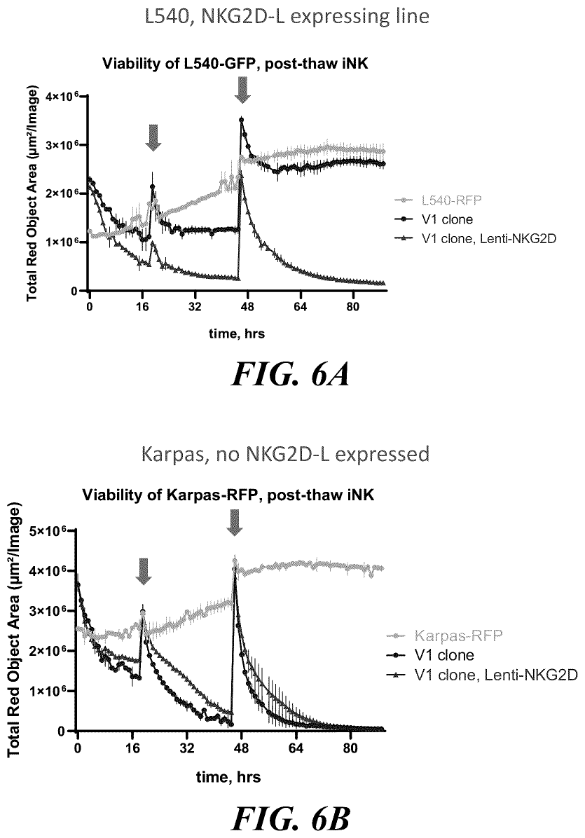

FIGS. 6A-B are graphs demonstrating L540 cell (FIG. 6A) and Karpas cell (FIG. 6B) killing by iNK cells overexpressing NKG2D. L540 cell line expressed NKG2D ligand while Karpas cell line did not express NKG2D ligand. The data indicates that NKG2D overexpression improves killing of NKG2D-ligand-expressing cancer cells.

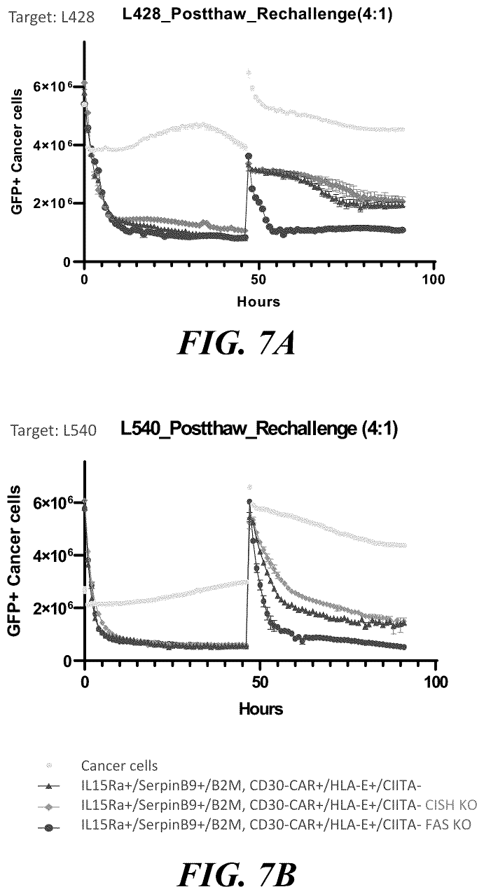

Next, the efficacy of FAS and CISH knock-out edits were re-evaluated in vitro. The FAS or CISH KO was introduced to a baseline clone comprising the edits of IL15/IL15Rα +/SERPINB9+/B2M knock-in and CD30−CAR+/HLA-E+/CIITA−. The edited iNK cells were evaluated in a killing assay against L428 cell line (FIG. 7A) and L540 cell line (FIG. 7B). Effector and target cell lines were plated at 4:1 effector:target (E:T) ratio. The data suggests that in both cell lines FAS KO iNK outperforms CISH KO iNK and the base line iNK with improved efficacy.

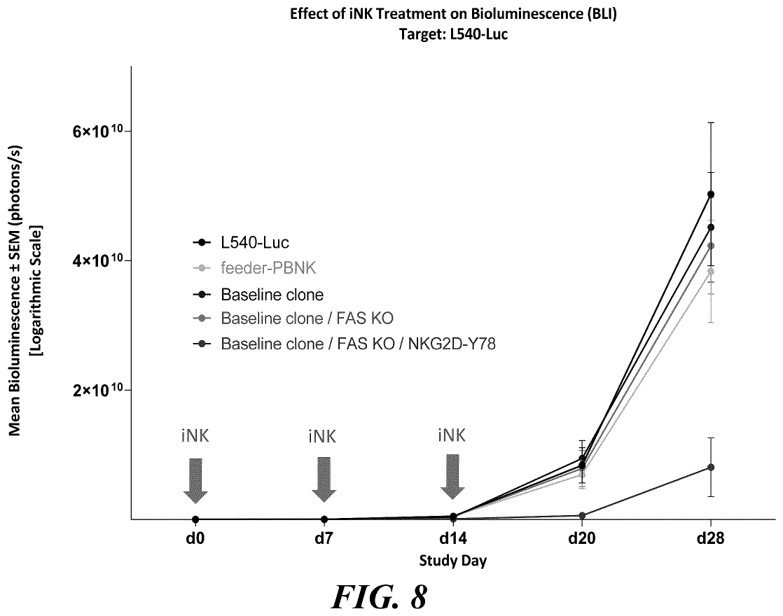

The therapeutic efficacy of iNK cells was further examined in vivo against L540 cancer line. Mice were intravenously injected with L540 cancer cells that also express luciferase. Mice were also injected with a dose of iNK cells comprising the edits shown in FIG. 8. FIG. 8 is a plot showing the in vivo efficacy against L540 cancer line by iNK cells comprising IL15/IL15Rα+/SERPINB9+/B2M− and CD30−CAR+/HLA-E+/CIITA−/FAS KG or IL15/IL15Rα +/SERPINB9+/B2M− and CD30−CAR+/HLA-E+/CIITA−/FAS KO/NKG2D-Y78 in comparison to baseline iNK cells comprising IL15/IL15Rα +/SERPINB9+/B2M− and CD30−CAR+/HLA-E+/CIITA- and to PBNK(control cells. The data suggests that the additional FAS KG in baseline iNK cells has improved efficacy in vitro but shows no improvement in vivo in liquid cancer, despite improved in vivo persistency. NKG2D overexpression protects iNK cells from exhaustion and improves killing of NKG2D-ligand-expressing cancer cells in vitro and in vivo.

### Sequences

Table 1 below provides exemplary sequences used to generate the gene edits described herein.

TABLE 1
Sequences

| | | | SEQ |
|---|---|---|---|
| Name | Description | Sequence | ID NO |
| CIITA | gRNA target | GGTCCATCTGGTCATAGAAG | 1, 49 |
| Ex3 T6 | sequence in | | |
| | CIITA, spacer | | |
| | sequence | | |
| CIITA | gRNA target | GCTCCAGGTAGCCACCTTCT | 2, 50 |
| Ex3 T16 | sequence in | | |
| | CIITA, spacer | | |
| | sequence | | |
| CIITA | gRNA target | TAGGGGCCCCAACTCCATGG | 3, 51 |
| Ex3 T20 | sequence in | | |
| | CIITA, spacer | | |
| | sequence | | |
| CIITA | gRNA target | GGCTTATGCCAATATCGGTG | 4, 52 |
| Ex4 T1 | sequence in | | |
| | CIITA, spacer | | |
| | sequence | | |
| CIITA | gRNA target | AGGTGATGAAGAGACCAGGG | 5, 53 |
| Ex4 T25 | sequence in | | |
| | CIITA, spacer | | |
| | sequence | | |
| CIITA F5 | Primer for | TCCTGACTCTCTGGTGTGAGAT | 6 |
| | CIITA | | |
| CIITA R5 | gRNA target | CAGAGAGCGTCCCACAGAC | 7 |
| | sequence in | | |
| | CIITA | | |
| B2M-2 | gRNA target | GGCCGAGATGTCTCGCTCCG | 8, 54 |
| gRNA | sequence in | | |
| (Exon | B2M, spacer | | |
| 1_T2) | sequence | | |
| FAS Ex1 | gRNA target | GGATTGCTCAACAACCATGC | 9, 55 |
| T7 | sequence in | | |
| | FAS, spacer | | |
| | sequence | | |
| FAS Ex1 | gRNA target | GATTGCTCAACAACCATGCT | 10, 56 |
| T9 | in FAS, spacer | | |
| | sequence | | |
| FAS Ex2 | gRNA target | GTGACTGACATCAACTCCAA | 11, 57 |
| T1 | in FAS, spacer | | |
| | sequence | | |
| FAS Ex2 | gRNA target | CACTTGGGCATTAACACTTT | 12, 58 |
| T2 | in FAS, spacer | | |
| | sequence | | |
| IL15 CDS | IL15 Coding | ggcattcatgtcttcattttgggctgtttcagtgc | 13 |
| | sequence | agggcttcctaaaacagaagccaactgggtgaatg | |
| | | taataagtgatttgaaaaaaattgaagatcttatt | |
| | | caatctatgcatattgatgctactttatatacgga | |
| | | aagtgatgttcaccccagttgcaaagtaacagcaa | |
| | | tgaagtgctttctcttggagttacaagttatttca | |
| | | cttgagtccggagatgcaagtattcatgatacagt | |
| | | agaaaatctgatcatcctagcaaacaacagtttgt | |
| | | cttctaatgggaatgtaacagaatctggatgcaaa | |
| | | gaatgtgaggaactggaggaaaaaaatattaaaga | |
| | | atttttgcagagttttgtacatattgtccaaatgt | |
| | | tcatcaacacttct | |
| IR15Rα | IL15Rα | atcacgtgccctccccccatgtccgtggaacacgc | 14 |
| CDS | coding | agacatctgggtcaagagctacagcttgtactcca | |
| | sequence | gggagcggtacatttgtaactctggtttcaagcgt | |
| | | aaagccggcacgtccagcctgacggagtgcgtgtt | |
| | | gaacaaggccacgaatgtcgcccactggacaaccc | |
| | | ccagtctcaaatgcattagagaccctgccctggtt | |
| | | caccaaaggccagcgccaccctccacagtaacgac | |
| | | ggcaggggtgaccccacagccagagagcctctccc | |
| | | cttctggaaaagagcccgcagcttcatctcccagc | |
| | | tcaaacaacacagcggccacaacagcagctattgt | |
| | | cccgggctcccagctgatgccttcaaaatcacctt | |
| | | ccacaggaaccacagagataagcagtcatgagtcc | |
| | | tcccacggcaccccctctcagacaacagccaagaa | |
| | | ctgggaactcacagcatccgcctcccaccagccgc | |
| | | caggtgtgtatccacagggccacagcgacaccact | |
| | | gtggctatctccacgtccactgtcctgctgtgtgg | |
| | | gctgagcgctgtgtctctcctggcatgctacctca | |
| | | agtcaaggcaaactcccccgctggccagcgttgaa | |
| | | atggaagccatggaggctctgccggtgacttgggg | |
| | | gaccagcagcagagatgaagacttggaaaactgct | |
| | | ctcaccaccta | |
| P2A | P2A | gctactaacttcagcctgctgaagcaggctggaga | 15 |
| | | cgtggaggagaaccctggacct | |
| HLA-G | HLA-G | gtcatggcgccccgaaccctcttcctg | 16 |
| peptide | peptide | | |
| B2M | B2M | atccagcgtactccaaagattcaggtttactcacg | 17 |
| | | tcatccagcagagaatggaaagtcaaatttcctga | |
| | | attgctatgtgtctgggtttcatccatccgacatt | |
| | | gaagttgacttactgaagaatggagagagaattga | |
| | | aaaagtggagcattcagacttgtctttcagcaagg | |
| | | actggtctttctatctcttgtactacactgaattc | |
| | | acccccactgaaaaagatgagtatgcctgccgtgt | |
| | | gaaccatgtgactttgtcacagcccaagatagtta | |
| | | agtgggatcgagacatg | |
| HLA-E | HLA-E coding | ggctcccactccttgaagtatttccacacttccgt | 18 |
| | sequence | gtcccggcccggccgcggggagccccgcttcatct | |
| | | ctgtgggctacgtggacgacacccagttcgtgcgc | |
| | | ttcgacaacgacgccgcgagtccgaggatggtgcc | |
| | | gcgggcgccgtggatggagcaggaggggtcagagt | |
| | | attgggaccgggagacacggagcgccagggacacc | |
| | | gcacagattttccgagtgaatctgcggacgctgcg | |
| | | cggctactacaatcagagcgaggccgggtctcaca | |
| | | ccctgcagtggatgcatggctgcgagctggggccc | |
| | | gacgggcgcttcctccgcgggtatgaacagttcgc | |
| | | ctacgacggcaaggattatctcaccctgaatgagg | |
| | | acctgcgctcctggaccgcggtggacacggcggct | |
| | | cagatctccgagcaaaagtcaaatgatgcctctga | |
| | | ggcggagcaccagagagcctacctggaagacacat | |
| | | gcgtggagtggctccacaaatacctggagaagggg | |
| | | aaggagacgctgcttcacctggagcccccaaagac | |
| | | acacgtgactcaccaccccatctctgaccatgagg | |
| | | ccaccctgaggtgctgggccctgggcttctaccct | |
| | | gcggagatcacactgacctggcagcaggatgggga | |
| | | gggccatacccaggacacggagctcgtggagacca | |
| | | ggcctgcaggggatggaaccttccagaagtgggca | |
| | | gctgtggtggtgccttctggagaggagcagagata | |
| | | cacgtgccatgtgcagcatgaggggctacccgagc | |
| | | ccgtcaccctgagatggaagccggcttcccagccc | |
| | | accatccccatcgtgggcatcattgctggcctggt | |
| | | tctccttggatctgtggtctctggagctgtggttg | |
| | | ctgctgtgatatggaggaagaagagctcaggtgga | |
| | | aaaggagggagctactctaaggctgagtggagcga | |
| | | cagtgcccaggggtctgagtctcacagcttg | |
| FAS Ex2 | gRNA target | TTGGAAGGCCTGCATCATGA | 19, 59 |
| T3 | in FAS, spacer | | |
| | sequence | | |
| FAS Ex2 | gRNA target | ACTCCAAGGGATTGGAATTG | 20, 60 |
| T7 | in FAS, spacer | | |
| | sequence | | |
| HLA-E | Coding | atgtctcgctccgttgccttagctgtgctcgcgct | 21 |
| Trimer | sequence | actctctctttctggattagaggctgtcatggcgc | |
| | | cccgaaccctcttcctgggtggaggcggttcaggc | |
| | | ggaggtggctctggcggtggcggatcgatccagcg | |
| | | tactccaaagattcaggtttactcacgtcatccag | |
| | | cagagaatggaaagtcaaatttcctgaattgctat | |
| | | gtgtctgggtttcatccatccgacattgaagttga | |
| | | cttactgaagaatggagagagaattgaaaaagtgg | |
| | | agcattcagacttgtctttcagcaaggactggtct | |
| | | ttctatctcttgtactacactgaattcacccccac | |
| | | tgaaaaagatgagtatgcctgccgtgtgaaccatg | |
| | | tgactttgtcacagcccaagatagttaagtgggat | |
| | | cgagacatgggtggtggtggttctggtggtggtgg | |
| | | ttctggcggcggcggctccggtggtggtggatccg | |
| | | gctcccactccttgaagtatttccacacttccgtg | |
| | | tcccggcccggccgcggggagccccgcttcatctc | |
| | | tgtgggctacgtggacgacacccagttcgtgcgct | |
| | | tcgacaacgacgccgcgagtccgaggatggtgccg | |
| | | cgggcgccgtggatggagcaggaggggtcagagta | |
| | | ttgggaccgggagacacggagcgccagggacaccg | |
| | | cacagattttccgagtgaatctgcggacgctgcgc | |
| | | ggctactacaatcagagcgaggccgggtctcacac | |
| | | cctgcagtggatgcatggctgcgagctggggcccg | |
| | | acgggcgcttcctccgcgggtatgaacagttcgcc | |
| | | tacgacggcaaggattatctcaccctgaatgagga | |
| | | cctgcgctcctggaccgcggtggacacggcggctc | |
| | | agatctccgagcaaaagtcaaatgatgcctctgag | |
| | | gcggagcaccagagagcctacctggaagacacatg | |
| | | cgtggagtggctccacaaatacctggagaagggga | |
| | | aggagacgctgcttcacctggagcccccaaagaca | |
| | | cacgtgactcaccaccccatctctgaccatgaggc | |
| | | caccctgaggtgctgggccctgggcttctaccctg | |
| | | cggagatcacactgacctggcagcaggatggggag | |
| | | ggccatacccaggacacggagctcgtggagaccag | |
| | | gcctgcaggggatggaaccttccagaagtgggcag | |
| | | ctgtggtggtgccttctggagaggagcagagatac | |
| | | acgtgccatgtgcagcatgaggggctacccgagcc | |
| | | cgtcaccctgagatggaagccggcttcccagccca | |
| | | ccatccccatcgtgggcatcattgctggcctggtt | |
| | | ctccttggatctgtggtctctggagctgtggttgc | |
| | | tgctgtgatatggaggaagaagagctcaggtggaa | |
| | | aaggagggagctactctaaggctgagtggagcgac | |
| | | agtgcccaggggtctgagtctcacagcttg | |
| IL15/IL15Rα | Coding | atggactggacctggatcctgttcctggtggccgc | 22 |
| fusion | sequence | cgccaccagggtgcacagcggcattcatgtcttca | |
| protein | | ttttgggctgtttcagtgcagggcttcctaaaaca | |
| | | gaagccaactgggtgaatgtaataagtgatttgaa | |
| | | aaaaattgaagatcttattcaatctatgcatattg | |
| | | atgctactttatatacggaaagtgatgttcacccc | |
| | | agttgcaaagtaacagcaatgaagtgctttctctt | |
| | | ggagttacaagttatttcacttgagtccggagatg | |
| | | caagtattcatgatacagtagaaaatctgatcatc | |
| | | ctagcaaacaacagtttgtcttctaatgggaatgt | |
| | | aacagaatctggatgcaaagaatgtgaggaactgg | |
| | | aggaaaaaaatattaaagaatttttgcagagtttt | |
| | | gtacatattgtccaaatgttcatcaacacttctag | |
| | | cggcggcggcagcggcggcggcggcagcggcggcg | |
| | | gcggcagcggcggcggcggcagcggcggcggcagc | |
| | | ctgcagatcacgtgccctccccccatgtccgtgga | |
| | | acacgcagacatctgggtcaagagctacagcttgt | |
| | | actccagggagcggtacatttgtaactctggtttc | |
| | | aagcgtaaagccggcacgtccagcctgacggagtg | |
| | | cgtgttgaacaaggccacgaatgtcgcccactgga | |
| | | caacccccagtctcaaatgcattagagaccctgcc | |
| | | ctggttcaccaaaggccagcgccaccctccacagt | |
| | | aacgacggcaggggtgaccccacagccagagagcc | |
| | | tctccccttctggaaaagagcccgcagcttcatct | |
| | | cccagctcaaacaacacagcggccacaacagcagc | |
| | | tattgtcccgggctcccagctgatgccttcaaaat | |
| | | caccttccacaggaaccacagagataagcagtcat | |
| | | gagtcctcccacggcaccccctctcagacaacagc | |
| | | caagaactgggaactcacagcatccgcctcccacc | |
| | | agccgccaggtgtgtatccacagggccacagcgac | |
| | | accactgtggctatctccacgtccactgtcctgct | |
| | | gtgtgggctgagcgctgtgtctctcctggcatgct | |
| | | acctcaagtcaaggcaaactcccccgctggccagc | |
| | | gttgaaatggaagccatggaggctctgccggtgac | |
| | | ttgggggaccagcagcagagatgaagacttggaaa | |
| | | actgctctcaccacctaggaagcgga | |
| IL15/IL15 | Coding | atggactggacctggatcctgttcctggtggccgc | 23 |
| Rα-P2A- | sequence | cgccaccagggtgcacagcggcattcatgtcttca | |
| HLA-E | | ttttgggctgtttcagtgcagggcttcctaaaaca | |
| trimer | | gaagccaactgggtgaatgtaataagtgatttgaa | |
| coding | | aaaaattgaagatcttattcaatctatgcatattg | |
| sequence | | atgctactttatatacggaaagtgatgttcacccc | |
| | | agttgcaaagtaacagcaatgaagtgctttctctt | |
| | | ggagttacaagttatttcacttgagtccggagatg | |
| | | caagtattcatgatacagtagaaaatctgatcatc | |
| | | ctagcaaacaacagtttgtcttctaatgggaatgt | |
| | | aacagaatctggatgcaaagaatgtgaggaactgg | |
| | | aggaaaaaaatattaaagaatttttgcagagtttt | |
| | | gtacatattgtccaaatgttcatcaacacttctag | |
| | | cggcggcggcagcggcggcggcggcagcggcggcg | |
| | | gcggcagcggcggcggcggcagcggcggcggcagc | |
| | | ctgcagatcacgtgccctccccccatgtccgtgga | |
| | | acacgcagacatctgggtcaagagctacagcttgt | |
| | | actccagggagcggtacatttgtaactctggtttc | |
| | | aagcgtaaagccggcacgtccagcctgacggagtg | |
| | | cgtgttgaacaaggccacgaatgtcgcccactgga | |
| | | caacccccagtctcaaatgcattagagaccctgcc | |
| | | ctggttcaccaaaggccagcgccaccctccacagt | |
| | | aacgacggcaggggtgaccccacagccagagagcc | |
| | | tctccccttctggaaaagagcccgcagcttcatct | |
| | | cccagctcaaacaacacagcggccacaacagcagc | |
| | | tattgtcccgggctcccagctgatgccttcaaaat | |
| | | caccttccacaggaaccacagagataagcagtcat | |
| | | gagtcctcccacggcaccccctctcagacaacagc | |
| | | caagaactgggaactcacagcatccgcctcccacc | |
| | | agccgccaggtgtgtatccacagggccacagcgac | |
| | | accactgtggctatctccacgtccactgtcctgct | |
| | | gtgtgggctgagcgctgtgtctctcctggcatgct | |
| | | acctcaagtcaaggcaaactcccccgctggccagc | |
| | | gttgaaatggaagccatggaggctctgccggtgac | |
| | | ttgggggaccagcagcagagatgaagacttggaaa | |
| | | actgctctcaccacctaggaagcggagctactaac | |
| | | ttcagcctgctgaagcaggctggagacgtggagga | |
| | | gaaccctggacctatgtctcgctccgttgccttag | |
| | | ctgtgctcgcgctactctctctttctggattagag | |
| | | gctgtcatggcgccccgaaccctcttcctgggtgg | |
| | | aggcggttcaggcggaggtggctctggcggtggcg | |
| | | gatcgatccagcgtactccaaagattcaggtttac | |
| | | tcacgtcatccagcagagaatggaaagtcaaattt | |
| | | cctgaattgctatgtgtctgggtttcatccatccg | |
| | | acattgaagttgacttactgaagaatggagagaga | |
| | | attgaaaaagtggagcattcagacttgtctttcag | |
| | | caaggactggtctttctatctcttgtactacactg | |
| | | aattcacccccactgaaaaagatgagtatgcctgc | |
| | | cgtgtgaaccatgtgactttgtcacagcccaagat | |
| | | agttaagtgggatcgagacatgggtggtggtggtt | |
| | | ctggtggtggtggttctggcggcggcggctccggt | |
| | | ggtggtggatccggctcccactccttgaagtattt | |
| | | ccacacttccgtgtcccggcccggccgcggggagc | |
| | | cccgcttcatctctgtgggctacgtggacgacacc | |
| | | cagttcgtgcgcttcgacaacgacgccgcgagtcc | |
| | | gaggatggtgccgcgggcgccgtggatggagcagg | |
| | | aggggtcagagtattgggaccgggagacacggagc | |
| | | gccagggacaccgcacagattttccgagtgaatct | |
| | | gcggacgctgcgcggctactacaatcagagcgagg | |
| | | ccgggtctcacaccctgcagtggatgcatggctgc | |
| | | gagctggggcccgacgggcgcttcctccgcgggta | |
| | | tgaacagttcgcctacgacggcaaggattatctca | |
| | | ccctgaatgaggacctgcgctcctggaccgcggtg | |
| | | gacacggcggctcagatctccgagcaaaagtcaaa | |
| | | tgatgcctctgaggcggagcaccagagagcctacc | |
| | | tggaagacacatgcgtggagtggctccacaaatac | |
| | | ctggagaaggggaaggagacgctgcttcacctgga | |
| | | gcccccaaagacacacgtgactcaccaccccatct | |
| | | ctgaccatgaggccaccctgaggtgctgggccctg | |
| | | ggcttctaccctgcggagatcacactgacctggca | |
| | | gcaggatggggagggccatacccaggacacggagc | |
| | | tcgtggagaccaggcctgcaggggatggaaccttc | |
| | | cagaagtgggcagctgtggtggtgccttctggaga | |
| | | ggagcagagatacacgtgccatgtgcagcatgagg | |
| | | ggctacccgagcccgtcaccctgagatggaagccg | |
| | | gcttcccagcccaccatccccatcgtgggcatcat | |
| | | tgctggcctggttctccttggatctgtggtctctg | |
| | | gagctgtggttgctgctgtgatatggaggaagaag | |
| | | agctcaggtggaaaaggagggagctactctaaggc | |
| | | tgagtggagcgacagtgcccaggggtctgagtctc | |
| | | acagcttg | |
| B2M-1 | gRNA target | GCTACTCTCTCTTTCTGGCC | 24, 61 |
| gRNA | in B2M | | |
| | PAM (TGG), | | |
| | spacer | | |
| | sequence | | |
| B2M-3 | gRNA target | CGCGAGCACAGCTAAGGCCA | 25, 62 |
| gRNA | in B2M | | |
| | PAM (CGG), | | |
| | spacer | | |
| | sequence | | |
| FAS Ex3 | gRNA target | CTAGGGACTGCACAGTCAAT | 26, 63 |
| T1 | in FAS, spacer | | |
| | sequence | | |
| CISH Ex1 | gRNA target | TCGCCGCTGCCGCGGGGACA | 27, 64 |
| T2 | in CISH, | | |
| | spacer | | |
| | sequence | | |
| CISH Ex1 | gRNA target | GACATGGTCCTCTGCGTTCA | 28, 65 |
| T18 | in CISH, | | |
| | spacer | | |
| | sequence | | |
| CISH Ex2 | gRNA target | GTCCGCTCCACAGCCAGCAA | 29, 66 |
| T1 | in CISH, | | |
| | spacer | | |
| | sequence | | |
| CISH Ex2 | gRNA target | GTTCCAGGGACGGGGCCCAC | 30, 67 |
| T2 | in CISH, | | |
| | spacer | | |
| | sequence | | |
| CISH Ex3 | gRNA target | TCGGGCCTCGCTGGCCGTAA | 31, 68 |
| T1 | in CISH, | | |
| | spacer | | |
| | sequence | | |
| CISH Ex3 | gRNA target | CGTACTAAGAACGTGCCTTC | 32, 69 |
| T2 | in CISH, | | |
| | spacer | | |
| | sequence | | |
| CISH Ex3 | gRNA target | GGGTTCCATTACGGCCAGCG | 33, 70 |
| T3 | in CISH, | | |
| | spacer | | |
| | sequence | | |
| CISH Ex3 | gRNA target | CAGGTGTTGTCGGGCCTCGC | 34, 71 |
| T5 | in CISH, | | |
| | spacer | | |
| | sequence | | |
| CISH Ex3 | gRNA target | TACTCAATGCGTACATTGGT | 35, 72 |
| T6 | in CISH, | | |
| | spacer | | |
| | sequence | | |
| CISH Ex3 | gRNA target | AAGGCTGACCACATCCGGAA | 36, 73 |
| T9 | in CISH, | | |
| | spacer | | |
| | sequence | | |
| CISH Ex3 | gRNA target | TACATTGGTGGGGCCACGAG | 37, 74 |
| T11 | in CISH, | | |
| | spacer | | |
| | sequence | | |
| CISH Ex3 | gRNA target | CTGTCAGTGAAAACCACTCG | 38, 75 |
| T14 | in CISH, | | |
| | spacer | | |
| | sequence | | |
| SERPIN | Coding | atggaaactctttctaatgcaagtggtacttttgc | 39 |
| B9 CDS | sequence | catacgccttttaaagatactgtgtcaagataacc | |
| | | cttcgcacaacgtgttctgttctcctgtgagcatc | |
| | | tcctctgccctggccatggttctcctaggggcaaa | |
| | | gggaaacaccgcaacccagatggcccaggcactgt | |
| | | ctttaaacacagaggaagacattcatcgggctttc | |
| | | cagtcgcttctcactgaagtgaacaaggctggcac | |
| | | acagtacctgctgagaacggccaacaggctctttg | |
| | | gagagaaaacttgtcagttcctctcaacgtttaag | |
| | | gaatcctgtcttcaattctaccatgctgagctgaa | |
| | | ggagctttcctttatcagagctgcagaagagtcca | |
| | | ggaaacacatcaacacctgggtctcaaaaaagacc | |
| | | gaaggtaaaattgaagagttgttgccgggtagctc | |
| | | aattgatgcagaaaccaggctggttcttgtcaatg | |
| | | ccatctacttcaaaggaaagtggaatgaaccgttt | |
| | | gacgaaacatacacaagggaaatgccctttaaaat | |
| | | aaaccaggaggagcaaaggccagtgcagatgatgt | |
| | | atcaggaggccacgtttaagctcgcccacgtgggc | |
| | | gaggtgcgcgcgcagctgctggagctgccctacgc | |
| | | caggaaggagctgagcctgctggtgctgctgcctg | |
| | | acgacggcgtggagctcagcacggtggaaaaaagt | |
| | | ctcacttttgagaaactcacagcctggaccaagcc | |
| | | agactgtatgaagagtactgaggttgaagttctcc | |
| | | ttccaaaatttaaactacaagaggattatgacatg | |
| | | gaatctgtgcttcggcatttgggaattgttgatgc | |
| | | cttccaacagggcaaggctgacttgtcggcaatgt | |
| | | cagcggagagagacctgtgtctgtccaagttcgtg | |
| | | cacaagagttttgtggaggtgaatgaagaaggcac | |
| | | cgaggcagcggcagcgtcgagctgctttgtagttg | |
| | | cagagtgctgcatggaatctggccccaggttctgt | |
| | | gctgaccaccctttccttttcttcatcaggcacaa | |
| | | cagagccaacagcattctgttctgtggcaggttct | |
| | | catcgcca | |
| SERPIN | | atggaaactctttctaatgcaagtggtacttttgc | 40 |
| B9-P2A- | | catacgccttttaaagatactgtgtcaagataacc | |
| HLA-E | | cttcgcacaacgtgttctgttctcctgtgagcatc | |
| transgene | | tcctctgccctggccatggttctcctaggggcaaa | |
| | | gggaaacaccgcaacccagatggcccaggcactgt | |
| | | ctttaaacacagaggaagacattcatcgggctttc | |
| | | cagtcgcttctcactgaagtgaacaaggctggcac | |
| | | acagtacctgctgagaacggccaacaggctctttg | |
| | | gagagaaaacttgtcagttcctctcaacgtttaag | |
| | | gaatcctgtcttcaattctaccatgctgagctgaa | |
| | | ggagctttcctttatcagagctgcagaagagtcca | |
| | | ggaaacacatcaacacctgggtctcaaaaaagacc | |
| | | gaaggtaaaattgaagagttgttgccgggtagctc | |
| | | aattgatgcagaaaccaggctggttcttgtcaatg | |
| | | ccatctacttcaaaggaaagtggaatgaaccgttt | |
| | | gacgaaacatacacaagggaaatgccctttaaaat | |
| | | aaaccaggaggagcaaaggccagtgcagatgatgt | |
| | | atcaggaggccacgtttaagctcgcccacgtgggc | |
| | | gaggtgcgcgcgcagctgctggagctgccctacgc | |
| | | caggaaggagctgagcctgctggtgctgctgcctg | |
| | | acgacggcgtggagctcagcacggtggaaaaaagt | |
| | | ctcacttttgagaaactcacagcctggaccaagcc | |
| | | agactgtatgaagagtactgaggttgaagttctcc | |
| | | ttccaaaatttaaactacaagaggattatgacatg | |
| | | gaatctgtgcttcggcatttgggaattgttgatgc | |
| | | cttccaacagggcaaggctgacttgtcggcaatgt | |
| | | cagcggagagagacctgtgtctgtccaagttcgtg | |
| | | cacaagagttttgtggaggtgaatgaagaaggcac | |
| | | cgaggcagcggcagcgtcgagctgctttgtagttg | |
| | | cagagtgctgcatggaatctggccccaggttctgt | |
| | | gctgaccaccctttccttttcttcatcaggcacaa | |
| | | cagagccaacagcattctgttctgtggcaggttct | |
| | | catcgccaggaagcggagctactaacttcagcctg | |
| | | ctgaagcaggctggagacgtggaggagaaccctgg | |
| | | acctatgtctcgctccgttgccttagctgtgctcg | |
| | | cgctactctctctttctggattagaggctgtcatg | |
| | | gcgccccgaaccctcttcctgggtggaggcggttc | |
| | | aggcggaggtggctctggcggtggcggatcgatcc | |
| | | agcgtactccaaagattcaggtttactcacgtcat | |
| | | ccagcagagaatggaaagtcaaatttcctgaattg | |
| | | ctatgtgtctgggtttcatccatccgacattgaag | |
| | | ttgacttactgaagaatggagagagaattgaaaaa | |
| | | gtggagcattcagacttgtctttcagcaaggactg | |
| | | gtctttctatctcttgtactacactgaattcaccc | |
| | | ccactgaaaaagatgagtatgcctgccgtgtgaac | |
| | | catgtgactttgtcacagcccaagatagttaagtg | |
| | | ggatcgagacatgggtggtggtggttctggtggtg | |
| | | gtggttctggcggcggcggctccggtggtggtgga | |
| | | tccggctcccactccttgaagtatttccacacttc | |
| | | cgtgtcccggcccggccgcggggagccccgcttca | |
| | | tctctgtgggctacgtggacgacacccagttcgtg | |
| | | cgcttcgacaacgacgccgcgagtccgaggatggt | |
| | | gccgcgggcgccgtggatggagcaggaggggtcag | |
| | | agtattgggaccgggagacacggagcgccagggac | |
| | | accgcacagattttccgagtgaatctgcggacgct | |
| | | gcgcggctactacaatcagagcgaggccgggtctc | |
| | | acaccctgcagtggatgcatggctgcgagctgggg | |
| | | cccgacgggcgcttcctccgcgggtatgaacagtt | |
| | | cgcctacgacggcaaggattatctcaccctgaatg | |
| | | aggacctgcgctcctggaccgcggtggacacgggg | |
| | | ctcagatctccgagcaaaagtcaaatgatgcctct | |
| | | gaggcggagcaccagagagcctacctggaagacac | |
| | | atgcgtggagtggctccacaaatacctggagaagg | |
| | | ggaaggagacgctgcttcacctggagcccccaaag | |
| | | acacacgtgactcaccaccccatctctgaccatga | |
| | | ggccaccctgaggtgctgggccctgggcttctacc | |
| | | ctgcggagatcacactgacctggcagcaggatggg | |
| | | gagggccatacccaggacacggagctcgtggagac | |
| | | caggcctgcaggggatggaaccttccagaagtggg | |
| | | cagctgtggtggtgccttctggagaggagcagaga | |
| | | tacacgtgccatgtgcagcatgaggggctacccga | |
| | | gcccgtcaccctgagatggaagccggcttcccagc | |
| | | ccaccatccccatcgtgggcatcattgctggcctg | |
| | | gttctccttggatctgtggtctctggagctgtggt | |
| | | tgctgctgtgatatggaggaagaagagctcaggtg | |
| | | gaaaaggagggagctactctaaggctgagtggagc | |
| | | gacagtgcccaggggtctgagtctcacagcttgta | |
| | | atgatagccgctgatcagcctcgactgtgccttct | |
| | | agttgccagccatctgttgtttgcccctcccccgt | |
| | | gccttccttgaccctggaaggtgccactcccactg | |
| | | tcctttcctaataaaa5501tgaggaaattgcatc | |
| | | gcattgtcgagtaggtgtcattctattctgggggg | |
| | | tggggtggggcaggacagcaagggggaggattggg | |
| | | aagacaatagcaggcatgctggggatgcggtgggc | |
| | | tctatgg | |
| SERPIN | | atggaaactctttctaatgcaagtggtacttttgc | 41 |
| B9-P2A- | | catacgccttttaaagatactgtgtcaagataacc | |
| IL15/IL15 | | cttcgcacaacgtgttctgttctcctgtgagcatc | |
| Rα | | tcctctgccctggccatggttctcctaggggcaaa | |
| transgene | | gggaaacaccgcaacccagatggcccaggcactgt | |
| | | ctttaaacacagaggaagacattcatcgggctttc | |
| | | cagtcgcttctcactgaagtgaacaaggctggcac | |
| | | acagtacctgctgagaacggccaacaggctctttg | |
| | | gagagaaaacttgtcagttcctctcaacgtttaag | |
| | | gaatcctgtcttcaattctaccatgctgagctgaa | |
| | | ggagctttcctttatcagagctgcagaagagtcca | |
| | | ggaaacacatcaacacctgggtctcaaaaaagacc | |
| | | gaaggtaaaattgaagagttgttgccgggtagctc | |
| | | aattgatgcagaaaccaggctggttcttgtcaatg | |
| | | ccatctacttcaaaggaaagtggaatgaaccgttt | |
| | | gacgaaacatacacaagggaaatgccctttaaaat | |
| | | aaaccaggaggagcaaaggccagtgcagatgatgt | |
| | | atcaggaggccacgtttaagctcgcccacgtgggc | |
| | | gaggtgcgcgcgcagctgctggagctgccctacgc | |
| | | caggaaggagctgagcctgctggtgctgctgcctg | |
| | | acgacggcgtggagctcagcacggtggaaaaaagt | |
| | | ctcacttttgagaaactcacagcctggaccaagcc | |
| | | agactgtatgaagagtactgaggttgaagttctcc | |
| | | ttccaaaatttaaactacaagaggattatgacatg | |
| | | gaatctgtgcttcggcatttgggaattgttgatgc | |
| | | cttccaacagggcaaggctgacttgtcggcaatgt | |
| | | cagcggagagagacctgtgtctgtccaagttcgtg | |
| | | cacaagagttttgtggaggtgaatgaagaaggcac | |
| | | cgaggcagcggcagcgtcgagctgctttgtagttg | |
| | | cagagtgctgcatggaatctggccccaggttctgt | |
| | | gctgaccaccctttccttttcttcatcaggcacaa | |
| | | cagagccaacagcattctgttctgtggcaggttct | |
| | | catcgccaggaagcggagctactaacttcagcctg | |
| | | ctgaagcaggctggagacgtggaggagaaccctgg | |
| | | acctatggactggacctggatcctgttcctggtgg | |
| | | ccgccgccaccagggtgcacagcggcattcatgtc | |
| | | ttcattttgggctgtttcagtgcagggcttcctaa | |
| | | aacagaagccaactgggtgaatgtaataagtgatt | |
| | | tgaaaaaaattgaagatcttattcaatctatgcat | |
| | | attgatgctactttatatacggaaagtgatgttca | |
| | | ccccagttgcaaagtaacagcaatgaagtgctttc | |
| | | tcttggagttacaagttatttcacttgagtccgga | |
| | | gatgcaagtattcatgatacagtagaaaatctgat | |
| | | catcctagcaaacaacagtttgtcttctaatggga | |
| | | atgtaacagaatctggatgcaaagaatgtgaggaa | |
| | | ctggaggaaaaaaatattaaagaatttttgcagag | |
| | | ttttgtacatattgtccaaatgttcatcaacactt | |
| | | ctagcggcggcggcagcggcggcggcggcagcggc | |
| | | ggcggcggcagcggcggcggcggcagcggcggcgg | |
| | | cagcctgcagatcacgtgccctccccccatgtccg | |
| | | tggaacacgcagacatctgggtcaagagctacagc | |
| | | ttgtactccagggagcggtacatttgtaactctgg | |
| | | tttcaagcgtaaagccggcacgtccagcctgacgg | |
| | | agtgcgtgttgaacaaggccacgaatgtcgcccac | |
| | | tggacaacccccagtctcaaatgcattagagaccc | |
| | | tgccctggttcaccaaaggccagcgccaccctcca | |
| | | cagtaacgacggcaggggtgaccccacagccagag | |
| | | agcctctccccttctggaaaagagcccgcagcttc | |
| | | atctcccagctcaaacaacacagcggccacaacag | |
| | | cagctattgtcccgggctcccagctgatgccttca | |
| | | aaatcaccttccacaggaaccacagagataagcag | |
| | | tcatgagtcctcccacggcaccccctctcagacaa | |
| | | cagccaagaactgggaactcacagcatccgcctcc | |
| | | caccagccgccaggtgtgtatccacagggccacag | |
| | | cgacaccactgtggctatctccacgtccactgtcc | |
| | | tgctgtgtgggctgagcgctgtgtctctcctggca | |
| | | tgctacctcaagtcaaggcaaactcccccgctggc | |
| | | cagcgttgaaatggaagccatggaggctctgccgg | |
| | | tgacttgggggaccagcagcagagatgaagacttg | |
| | | gaaaactgctctcaccacctatgataaccgctgat | |
| | | cagcctcgactgtgccttctagttgccagccatct | |
| | | gttgtttgcccctcccccgtgccttccttgaccct | |
| | | ggaaggtgccactcccactgtcctttcctaataaa | |
| | | atgaggaaattgcatcgcattgtctgagtaggtgt | |
| | | cattctattctggggggggggtggggcaggacagc | |
| | | aagggggaggattgggaagacaatagcaggcatgc | |
| | | tggggatgcggtgggctctatgg | |
| HLA-E | Amino acid | MSRSVALAVLALLSLSGLEAVMAPRTLFLGGGGSG | 42 |
| trimer | sequence | GGGSGGGGSIQRTPKIQVYSRHPAENGKSNFLNCY | |
| | | VSGFHPSDIEVDLLKNGERIEKVEHSDLSFSKDWS | |
| | | FYLLYYTEFTPTEKDEYACRVNHVTLSQPKIVKWD | |
| | | RDMGGGGSGGGGSGGGGSGGGGSGSHSLKYFHTSV | |
| | | SRPGRGEPRFISVGYVDDTQFVRFDNDAASPRMVP | |
| | | RAPWMEQEGSEYWDRETRSARDTAQIFRVNLRTLR | |
| | | GYYNQSEAGSHTLQWMHGCELGPDGRFLRGYEQFA | |
| | | YDGKDYLTLNEDLRSWTAVDTAAQISEQKSNDASE | |
| | | AEHQRAYLEDTCVEWLHKYLEKGKETLLHLEPPKT | |
| | | HVTHHPISDHEATLRCWALGFYPAEITLTWQQDGE | |
| | | GHTQDTELVETRPAGDGTFQKWAAVVVPSGEEQRY | |
| | | TCHVQHEGLPEPVTLRWKPASQPTIPIVGIIAGLV | |
| | | LLGSVVSGAVVAAVIWRKKSSGGKGGSYSKAEWSD | |
| | | SAQGSESHSL | |
| IL15/IL15 | Amino acid | MDWTWILFLVAAATRVHSGIHVFILGCFSAGLPKT | 43 |
| Rαfusion | sequence | EANWVNVISDLKKIEDLIQSMHIDATLYTESDVHP | |
| | | SCKVTAMKCFLLELQVISLESGDASIHDTVENLII | |
| | | LANNSLSSNGNVTESGCKECEELEEKNIKEFLQSF | |
| | | VHIVQMFINTSSGGGSGGGGSGGGGSGGGGSGGGS | |
| | | LQITCPPPMSVEHADIWVKSYSLYSRERYICNSGF | |
| | | KRKAGTSSLTECVLNKATNVAHWTTPSLKCIRDPA | |
| | | LVHQRPAPPSTVTTAGVTPQPESLSPSGKEPAASS | |
| | | PSSNNTAATTAAIVPGSQLMPSKSPSTGTTEISSH | |
| | | ESSHGTPSQTTAKNWELTASASHQPPGVYPQGHSD | |
| | | TTVAISTSTVLLCGLSAVSLLACYLKSRQTPPLAS | |
| | | VEMEAMEALPVTWGTSSRDEDLENCSHHLGSG | |
| SERPINB9 | Amino acid | METLSNASGTFAIRLLKILCQDNPSHNVFCSPVSI | 44 |
| | sequence | SSALAMVLLGAKGNTATQMAQALSLNTEEDIHRAF | |
| | | QSLLTEVNKAGTQYLLRTANRLFGEKTCQFLSTFK | |
| | | ESCLQFYHAELKELSFIRAAEESRKHINTWVSKKT | |
| | | EGKIEELLPGSSIDAETRLVLVNAIYFKGKWNEPF | |
| | | DETYTREMPFKINQEEQRPVQMMYQEATFKLAHVG | |
| | | EVRAQLLELPYARKELSLLVLLPDDGVELSTVEKS | |
| | | LTFEKLTAWTKPDCMKSTEVEVLLPKFKLQEDYDM | |
| | | ESVLRHLGIVDAFQQGKADLSAMSAERDLCLSKFV | |
| | | HKSFVEVNEEGTEAAAASSCFVVAECCMESGPRFC | |
| | | ADHPFLFFIRHNRANSILFCGRFSSP | |
| GPC3 | Nucleic Acid | ATGGCGCTTCCGGTGACAGCACTGCTCCTCCCCTT | 45 |
| CAR | Sequence | GGCGCTGTTGCTCCACGCAGCAAGGCCGCAAGTGC | |
| | | AGCTCGTGCAAAGCGGCGGCGGACTGGTGCAACCC | |
| | | GGCGGATCTCTGAGACTGAGCTGCGCCGCCAGCTA | |
| | | CTTCGACTTCGACAGCTACGAGATGAGCTGGGTGA | |
| | | GGCAAGCCCCCGGAAAAGGACTGGAGTGGATCGGC | |
| | | AGCATCTACCACAGCGGCTCCACCTACTACAACCC | |
| | | CTCTCTGAAATCTAGAGTGACCATCTCTAGAGACA | |
| | | ACTCCAAGAACACACTGTACCTCCAGATGAACACA | |
| | | CTGAGAGCCGAGGACACCGCCACCTATTACTGCGC | |
| | | TAGAGTGAACATGGACAGATTCGACTACTGGGGCC | |
| | | AAGGCACACTGGTGACAGTGAGCTCCAGTGCTGCT | |
| | | GCCTTTGTCCCGGTATTTCTCCCAGCCAAACCGAC | |
| | | CACGACTCCCGCCCCGCGCCCTCCGACACCCGCTC | |
| | | CCACCATCGCCTCTCAACCTCTTAGTCTTCGCCCC | |
| | | GAGGCATGCCGACCCGCCGCCGGGGGTGCTGTTCA | |
| | | TACGAGGGGCTTGGACTTCGCTTGTGATATTTACA | |
| | | TTTGGGCTCCGTTGGCGGGTACGTGCGGCGTCCTT | |
| | | TTGTTGTCACTCGTTATTACTTTGTATTGTAATCA | |
| | | CAGGAATCGCTCAAAGCGGAGTAGGTTGTTGCATT | |
| | | CCGATTACATGAATATGACTCCTCGCCGGCCTGGG | |
| | | CCGACAAGAAAACATTACCAACCCTATGCCCCCCC | |
| | | ACGAGACTTCGCTGCGTACAGGTCCCGAGTGAAGT | |
| | | TTTCCCGAAGCGCAGACGCTCCGGCATATCAGCAA | |
| | | GGACAGAATCAGCTGTATAACGAACTGAATTTGGG | |
| | | ACGCCGCGAGGAGTATGACGTGCTTGATAAACGCC | |
| | | GGGGGAGAGACCCGGAAATGGGGGGTAAACCCCGA | |
| | | AGAAAGAATCCCCAAGAAGGACTCTACAATGAACT | |
| | | CCAGAAGGATAAGATGGCGGAGGCCTACTCAGAAA | |
| | | TAGGTATGAAGGGCGAACGACGACGGGGAAAAGGT | |
| | | CACGATGGCCTCTACCAAGGGTTGAGTACGGCAAC | |
| | | CAAAGATACGTACGATGCACTGCATATGCAGGCCC | |
| | | TGCCTCCCAGA | |
| GPC3 | Amino Acid | MALPVTALLLPLALLLHAARPQVQLVQSGGGLVQP | 46 |
| CAR | Sequence | GGSLRLSCAASYFDFDSYEMSWVRQAPGKGLEWIG | |
| | | SIYHSGSTYYNPSLKSRVTISRDNSKNTLYLQMNT | |
| | | LRAEDTATYYCARVNMDRFDYWGQGTLVTVSSSAA | |
| | | AFVPVFLPAKPTTTPAPRPPTPAPTIASQPLSLRP | |
| | | EACRPAAGGAVHTRGLDFACDIYIWAPLAGTCGVL | |
| | | LLSLVITLYCNHRNRSKRSRLLHSDYMNMTPRRPG | |
| | | PTRKHYQPYAPPRDFAAYRSRVKFSRSADAPAYQQ | |
| | | GQNQLYNELNLGRREEYDVLDKRRGRDPEMGGKPR | |
| | | RKNPQEGLYNELQKDKMAEAYSEIGMKGERRRGKG | |
| | | HDGLYQGLSTATKDTYDALHMQALPPR | |
| GPR87 | Nucleic Acid | ATGGCCTTACCAGTGACCGCCTTGCTCCTGCCGCT | 47 |
| CAR | Sequence | GGCCTTGCTGCTCCACGCCGCCAGGCCGGACGTGG | |
| | | TCATGACCCAGACCCCTCTGAGCCTGCCCGTGAGC | |
| | | CTGGGCGACCAGGCTTCCATCTCCTGCAGAAGCTC | |
| | | CCAGAGCCTGGTGCACAGCAGCGGCAACACCTACC | |
| | | TGCACTGGTACCTGCAGAAGCCCGGCCAGAGCCCC | |
| | | AAGCTGCTGATCTACAAGGTGTCCAACAGATTTTC | |
| | | CGGCGTGCCCGACAGATTTTCCGGATCCGGCAGCG | |
| | | GCACCGACTTCACACTGAAGATCAGCAGAGTGGAG | |
| | | GCCGAGGACCTGGGCGTGTACTTCTGTAGCCAGAG | |
| | | CACACACGTGCCTTACACCTTTGGCGGCGGCACAA | |
| | | AGCTGGAGATCAAGGGCGGCGGCGGCTCCGGAGGA | |
| | | GGAGGAAGCGGAGGAGGCGGCTCCGAGGTGCAGCT | |
| | | GCAGCAGTCCGGCCCTGACCTGGTGAAGCCTGGCG | |
| | | CCTCCATGAAGATCAGCTGCAAGGCCAGCGGCTAC | |
| | | TCCTTCACCGATTACACCATGCACTGGGTGAAGCA | |
| | | GAGCCACGGCAAGAATTTCGAGTGGATCGGCCTGA | |
| | | TCAATCCCTACAATGACGGCACCACCTACAATCAG | |
| | | AAGTTCAAGGGCAAGGCCACACTGACAGTGGATAA | |
| | | GAGCAGCTCCACAGCCTACATGGAGCTGCTGAGCC | |
| | | TGACAAGCGAGGATTCCGCCGTGTACTACTGCGCC | |
| | | TCCCTGGACTACTGGGGCCAGGGCACCTCCGTGAC | |
| | | CGTGTCCTCCACCACTACCCCAGCACCGAGGCCAC | |
| | | CCACCCCGGCTCCTACCATCGCCTCCCAGCCTCTG | |
| | | TCCCTGCGTCCGGAGGCATGTAGACCCGCAGCTGG | |
| | | TGGGGCCGTGCATACCCGGGGTCTTGACTTCGCCT | |
| | | GCGATTTCTGGGTGCTGGTCGTTGTGGGCGGCGTG | |
| | | CTGGCCTGCTACAGCCTGCTGGTGACAGTGGCCTT | |
| | | CATCATCTTTTGGGTGAGGAGCAAGCGGAGCAGAC | |
| | | TGCTGCACAGCGACTACATGAACATGACCCCCCGG | |
| | | AGGCCTGGCCCCACCCGGAAGCACTACCAGCCCTA | |
| | | CGCCCCTCCCAGGGATTTCGCCGCCTACCGGAGCC | |
| | | GCGTGAAATTCAGCCGCAGCGCAGATGCTCCAGCC | |
| | | TACAAGCAGGGGCAGAACCAGCTCTACAACGAACT | |
| | | CAATCTTGGTCGGAGAGAGGAGTACGACGTGCTGG | |
| | | ACAAGCGGAGAGGACGGGACCCAGAAATGGGCGGG | |
| | | AAGCCGCGCAGAAAGAATCCCCAAGAGGGCCTGTA | |
| | | CAACGAGCTCCAAAAGGATAAGATGGCAGAAGCCT | |
| | | ATAGCGAGATTGGTATGAAAGGGGAACGCAGAAGA | |
| | | GGCAAAGGCCACGACGGACTGTACCAGGGACTCAG | |
| | | CACCGCCACCAAGGACACCTATGACGCTCTTCACA | |
| | | TGCAGGCCCTGCCGCCTCGG | |
| GPR87 | Amino acid | MALPVTALLLPLALLLHAARPDVVMTQTPLSLPVS | 48 |
| CAR | sequence | LGDQASISCRSSQSLVHSSGNTYLHWYLQKPGQSP | |
| | | KLLIYKVSNRFSGVPDRFSGSGSGTDFTLKISRVE | |
| | | AEDLGVYFCSQSTHVPYTFGGGTKLEIKGGGGSGG | |
| | | GGSGGGGSEVQLQQSGPDLVKPGASMKISCKASGY | |
| | | SFTDYTMHWVKQSHGKNFEWILINPYNDGTTYNQK | |
| | | FKGKATLTVDKSSSTAYMELLSLTSEDSAVYYCAS | |
| | | LDYWGQGTSVTVSSTTTPAPRPPTPAPTIASQPLS | |
| | | LRPEACRPAAGGAVHTRGLDFACDFWVLVVVGGVL | |
| | | ACYSLLVTVAFIIFWVRSKRSRLLHSDYMNMTPRR | |
| | | PGPTRKHYQPYAPPRDFAAYRSRVKFSRSADAPAY | |
| | | KQGQNQLYNELNLGRREEYDVLDKRRGRDPEMGGK | |
| | | PRRKNPQEGLYNELQKDKMAEAYSEIGMKGERRRG | |
| | | KGHDGLYQGLSTATKDTYDALHMQALPPR | |

### Terminology

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A genetically modified cell, comprising:
a disrupted FAS gene;
a disrupted B2M gene;
an insertion of a polynucleotide encoding a fusion of IL15 and IL15Rα (IL15/IL15Rα) in the disrupted B2M gene; and
an insertion of a polynucleotide encoding a CAR, wherein the cell expresses the IL15/IL15Rα fusion protein and the CAR, and the cell has disrupted expressions of FAS.

2. The genetically modified cell of claim 1, wherein the CAR is an anti-GPR87 CAR.

3. The genetically modified cell of claim 1, wherein the cell comprises a disrupted CIITA gene, and the polynucleotide encoding the CAR is inserted into the disrupted CIITA gene.

4. The genetically modified cell of claim 1, wherein the genetically modified cell comprises a disrupted CISH gene.

5. The genetically modified cell of claim 1, wherein the genetically modified cell does not comprise a disrupted CISH gene.

6. The genetically modified cell of claim 2, wherein the polynucleotide encoding the anti-GPR87 CAR comprises the sequence of SEQ ID NO: 47.

7. The genetically modified cell of claim 1, wherein the genetically modified cell does not comprise:
a genetic modification of a major histocompatibility complex (MHC) gene or a transcriptional regulator gene thereof;
an insertion of a polynucleotide encoding HLA-E, an insertion of a polynucleotide encoding SERPINB9, or both; and/or
a disrupted CIITA gene.

8. (canceled)

9. (canceled)

10. The genetically modified cell of claim 1, wherein the genetically modified cell is a stem cell.

11. The genetically modified cell of claim 10, wherein the stem cell is an induced pluripotent stem cell (iPSC), a hematopoietic stem cell, an embryonic stem cell, or an adult stem cell.

12. The genetically modified cell of claim 1, wherein the genetically modified cell is a genome-edited iPSC.

13. The genetically modified cell of claim 1, wherein the genetically modified cell is a natural killer (NK) cell obtained from a genome-edited iPSC.

14. The genetically modified cell of claim 1, wherein the genetically modified cell is a differentiated cell or a somatic cell.

15. The genetically modified cell of claim 1, wherein the genetically modified cell is capable of being differentiated into lineage-restricted progenitor cells or fully differentiated somatic cells.

16. The genetically modified cell of claim 1, wherein the genetically modified cell is a natural killer (NK) cell.

17. The genetically modified cell of claim 16, wherein the NK cell has been differentiated from a genome-edited iPSC, wherein the NK cell comprises the genome edits of the genome-edited iPSC, and wherein the NK cell has not been genome-edited after the differentiation.

18. The genetically modified cell of claim 1, wherein the genetically modified cell is capable of cell expansion in the absence of exogenous IL15 in cell culture media.

19. (canceled)

20. A population of cells, comprising lineage-restricted progenitor cells or fully differentiated somatic cells derived from one or more genetically modified cells comprising:
a disrupted FAS gene;
a disrupted B2M gene;
an insertion of a polynucleotide encoding a fusion of IL15 and IL15Rα (IL15/IL15Rα) in the disrupted B2M gene; and
an insertion of a polynucleotide encoding a CAR, wherein the cell expresses the IL15/IL15Rα fusion protein and the CAR, and the cell has disrupted expressions of FAS.

21.-34. (canceled)

35. A method for treating a subject in need thereof, the method comprising:
(a) obtaining or having obtained a population of cells comprising lineage-restricted progenitor cells or fully differentiated somatic cells derived from one or more genetically modified cells comprising:
a disrupted FAS gene;
a disrupted B2M gene;
an insertion of a polynucleotide encoding a fusion of IL15 and IL15Rα (IL15/IL15Rα) in the disrupted B2M gene; and
an insertion of a polynucleotide encoding a CAR, wherein the cell expresses the IL15/IL15Rα fusion protein and the CAR, and the cell has disrupted expressions of FAS; and
(b) administering the lineage-restricted progenitor cells or fully differentiated somatic cells to the subject.

36. (canceled)

37. The method of claim 35, wherein the fully differentiated somatic cells are NK cells.

38. The method of claim 35, wherein the subject has, is suspected of having, or is at risk for a cancer; optionally the subject is human.

39.-51. (canceled)


SEQUENCE LISTING (80 sequences)

SEQ ID NO: 1 - DNA, 20 bp, synthetic construct
  source 1..20: mol_type = other DNA
ggtccatctggtcatagaag

SEQ ID NO: 2 - DNA, 20 bp, synthetic construct
  source 1..20: mol_type = other DNA
gctccaggtagccaccttct

SEQ ID NO: 3 - DNA, 20 bp, synthetic construct
  source 1..20: mol_type = other DNA
taggggccccaactccatgg

SEQ ID NO: 4 - DNA, 20 bp, synthetic construct
  source 1..20: mol_type = other DNA
ggcttatgccaatatcggtg

SEQ ID NO: 5 - DNA, 20 bp, synthetic construct
  source 1..20: mol_type = other DNA
aggtgatgaagagaccaggg

SEQ ID NO: 6 - DNA, 22 bp, synthetic construct
  source 1..22: mol_type = other DNA
tcctgactctctggtgtgagat

SEQ ID NO: 7 - DNA, 19 bp, synthetic construct
  source 1..19: mol_type = other DNA
cagagagcgtcccacagac

SEQ ID NO: 8 - DNA, 20 bp, synthetic construct
  source 1..20: mol_type = other DNA
ggccgagatgtctcgctccg

SEQ ID NO: 9 - DNA, 20 bp, synthetic construct
  source 1..20: mol_type = other DNA
ggattgctcaacaaccatgc

SEQ ID NO: 10 - DNA, 20 bp, synthetic construct
  source 1..20: mol_type = other DNA
gattgctcaacaaccatgct

SEQ ID NO: 11 - DNA, 20 bp, synthetic construct
  source 1..20: mol_type = other DNA
gtgactgacatcaactccaa

SEQ ID NO: 12 - DNA, 20 bp, synthetic construct
  source 1..20: mol_type = other DNA
cacttgggcattaacacttt

SEQ ID NO: 13 - DNA, 399 bp, synthetic construct
  source 1..399: mol_type = other DNA
ggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttct

SEQ ID NO: 14 - DNA, 711 bp, synthetic construct
  source 1..711: mol_type = other DNA
atcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccaccta

SEQ ID NO: 15 - DNA, 57 bp, synthetic construct
  source 1..57: mol_type = other DNA
gctactaacttcagcctgctgaagcaggctggagacgtggaggagaaccctggacct

SEQ ID NO: 16 - DNA, 27 bp, synthetic construct
  source 1..27: mol_type = other DNA
gtcatggcgccccgaaccctcttcctg

SEQ ID NO: 17 - DNA, 297 bp, synthetic construct
  source 1..297: mol_type = other DNA
atccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatg

SEQ ID NO: 18 - DNA, 1011 bp, synthetic construct
  source 1..1011: mol_type = other DNA
ggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttg

SEQ ID NO: 19 - DNA, 20 bp, synthetic construct
  source 1..20: mol_type = other DNA
ttggaaggcctgcatcatga

SEQ ID NO: 20 - DNA, 20 bp, synthetic construct
  source 1..20: mol_type = other DNA
actccaagggattggaattg

SEQ ID NO: 21 - DNA, 1500 bp, synthetic construct
  source 1..1500: mol_type = other DNA
atgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttg

SEQ ID NO: 22 - DNA, 1251 bp, synthetic construct
  source 1..1251: mol_type = other DNA
atggactggacctggatcctgttcctggtggccgccgccaccagggtgcacagcggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctagcggcggcggcagcggcggcggcggcagcggcggcggcggcagcggcggcggcggcagcggcggcggcagcctgcagatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccacctaggaagcgga

SEQ ID NO: 23 - DNA, 2808 bp, synthetic construct
  source 1..2808: mol_type = other DNA
atggactggacctggatcctgttcctggtggccgccgccaccagggtgcacagcggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctagcggcggcggcagcggcggcggcggcagcggcggcggcggcagcggcggcggcggcagcggcggcggcagcctgcagatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccacctaggaagcggagctactaacttcagcctgctgaagcaggctggagacgtggaggagaaccctggacctatgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttg

SEQ ID NO: 24 - DNA, 20 bp, synthetic construct
  source 1..20: mol_type = other DNA
gctactctctctttctggcc

SEQ ID NO: 25 - DNA, 20 bp, synthetic construct
  source 1..20: mol_type = other DNA
cgcgagcacagctaaggcca

SEQ ID NO: 26 - DNA, 20 bp, synthetic construct
  source 1..20: mol_type = other DNA
ctagggactgcacagtcaat

SEQ ID NO: 27 - DNA, 20 bp, synthetic construct
  source 1..20: mol_type = other DNA
tcgccgctgccgcggggaca

SEQ ID NO: 28 - DNA, 20 bp, synthetic construct
  source 1..20: mol_type = other DNA
gacatggtcctctgcgttca

SEQ ID NO: 29 - DNA, 20 bp, synthetic construct
  source 1..20: mol_type = other DNA
gtccgctccacagccagcaa

SEQ ID NO: 30 - DNA, 20 bp, synthetic construct
  source 1..20: mol_type = other DNA
gttccagggacggggcccac

SEQ ID NO: 31 - DNA, 20 bp, synthetic construct
  source 1..20: mol_type = other DNA
tcgggcctcgctggccgtaa

SEQ ID NO: 32 - DNA, 20 bp, synthetic construct
  source 1..20: mol_type = other DNA
cgtactaagaacgtgccttc

SEQ ID NO: 33 - DNA, 20 bp, synthetic construct
  source 1..20: mol_type = other DNA
gggttccattacggccagcg

SEQ ID NO: 34 - DNA, 20 bp, synthetic construct
  source 1..20: mol_type = other DNA
caggtgttgtcgggcctcgc

SEQ ID NO: 35 - DNA, 20 bp, synthetic construct
  source 1..20: mol_type = other DNA
tactcaatgcgtacattggt

SEQ ID NO: 36 - DNA, 20 bp, synthetic construct
  source 1..20: mol_type = other DNA
aaggctgaccacatccggaa

SEQ ID NO: 37 - DNA, 20 bp, synthetic construct
  source 1..20: mol_type = other DNA
tacattggtggggccacgag

SEQ ID NO: 38 - DNA, 20 bp, synthetic construct
  source 1..20: mol_type = other DNA
ctgtcagtgaaaaccactcg

SEQ ID NO: 39 - DNA, 1128 bp, synthetic construct
  source 1..1128: mol_type = other DNA
atggaaactctttctaatgcaagtggtacttttgccatacgccttttaaagatactgtgtcaagataacccttcgcacaacgtgttctgttctcctgtgagcatctcctctgccctggccatggttctcctaggggcaaagggaaacaccgcaacccagatggcccaggcactgtctttaaacacagaggaagacattcatcgggctttccagtcgcttctcactgaagtgaacaaggctggcacacagtacctgctgagaacggccaacaggctctttggagagaaaacttgtcagttcctctcaacgtttaaggaatcctgtcttcaattctaccatgctgagctgaaggagctttcctttatcagagctgcagaagagtccaggaaacacatcaacacctgggtctcaaaaaagaccgaaggtaaaattgaagagttgttgccgggtagctcaattgatgcagaaaccaggctggttcttgtcaatgccatctacttcaaaggaaagtggaatgaaccgtttgacgaaacatacacaagggaaatgccctttaaaataaaccaggaggagcaaaggccagtgcagatgatgtatcaggaggccacgtttaagctcgcccacgtgggcgaggtgcgcgcgcagctgctggagctgccctacgccaggaaggagctgagcctgctggtgctgctgcctgacgacggcgtggagctcagcacggtggaaaaaagtctcacttttgagaaactcacagcctggaccaagccagactgtatgaagagtactgaggttgaagttctccttccaaaatttaaactacaagaggattatgacatggaatctgtgcttcggcatttgggaattgttgatgccttccaacagggcaaggctgacttgtcggcaatgtcagcggagagagacctgtgtctgtccaagttcgtgcacaagagttttgtggaggtgaatgaagaaggcaccgaggcagcggcagcgtcgagctgctttgtagttgcagagtgctgcatggaatctggccccaggttctgtgctgaccaccctttccttttcttcatcaggcacaacagagccaacagcattctgttctgtggcaggttctcatcgcca

SEQ ID NO: 40 - DNA, 2944 bp, synthetic construct
  source 1..2944: mol_type = other DNA
atggaaactctttctaatgcaagtggtacttttgccatacgccttttaaagatactgtgtcaagataacccttcgcacaacgtgttctgttctcctgtgagcatctcctctgccctggccatggttctcctaggggcaaagggaaacaccgcaacccagatggcccaggcactgtctttaaacacagaggaagacattcatcgggctttccagtcgcttctcactgaagtgaacaaggctggcacacagtacctgctgagaacggccaacaggctctttggagagaaaacttgtcagttcctctcaacgtttaaggaatcctgtcttcaattctaccatgctgagctgaaggagctttcctttatcagagctgcagaagagtccaggaaacacatcaacacctgggtctcaaaaaagaccgaaggtaaaattgaagagttgttgccgggtagctcaattgatgcagaaaccaggctggttcttgtcaatgccatctacttcaaaggaaagtggaatgaaccgtttgacgaaacatacacaagggaaatgccctttaaaataaaccaggaggagcaaaggccagtgcagatgatgtatcaggaggccacgtttaagctcgcccacgtgggcgaggtgcgcgcgcagctgctggagctgccctacgccaggaaggagctgagcctgctggtgctgctgcctgacgacggcgtggagctcagcacggtggaaaaaagtctcacttttgagaaactcacagcctggaccaagccagactgtatgaagagtactgaggttgaagttctccttccaaaatttaaactacaagaggattatgacatggaatctgtgcttcggcatttgggaattgttgatgccttccaacagggcaaggctgacttgtcggcaatgtcagcggagagagacctgtgtctgtccaagttcgtgcacaagagttttgtggaggtgaatgaagaaggcaccgaggcagcggcagcgtcgagctgctttgtagttgcagagtgctgcatggaatctggccccaggttctgtgctgaccaccctttccttttcttcatcaggcacaacagagccaacagcattctgttctgtggcaggttctcatcgccaggaagcggagctactaacttcagcctgctgaagcaggctggagacgtggaggagaaccctggacctatgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaatgatagccgctgatcagcctcgactgtgccttctagttgccagccatctgttgtttgcccctcccccgtgccttccttgaccctggaaggtgccactcccactgtcctttcctaataaaatgaggaaattgcatcgcattgtcgagtaggtgtcattctattctggggggtggggtggggcaggacagcaagggggaggattgggaagacaatagcaggcatgctggggatgcggtgggctctatgg

SEQ ID NO: 41 - DNA, 2684 bp, synthetic construct
  source 1..2684: mol_type = other DNA
atggaaactctttctaatgcaagtggtacttttgccatacgccttttaaagatactgtgtcaagataacccttcgcacaacgtgttctgttctcctgtgagcatctcctctgccctggccatggttctcctaggggcaaagggaaacaccgcaacccagatggcccaggcactgtctttaaacacagaggaagacattcatcgggctttccagtcgcttctcactgaagtgaacaaggctggcacacagtacctgctgagaacggccaacaggctctttggagagaaaacttgtcagttcctctcaacgtttaaggaatcctgtcttcaattctaccatgctgagctgaaggagctttcctttatcagagctgcagaagagtccaggaaacacatcaacacctgggtctcaaaaaagaccgaaggtaaaattgaagagttgttgccgggtagctcaattgatgcagaaaccaggctggttcttgtcaatgccatctacttcaaaggaaagtggaatgaaccgtttgacgaaacatacacaagggaaatgccctttaaaataaaccaggaggagcaaaggccagtgcagatgatgtatcaggaggccacgtttaagctcgcccacgtgggcgaggtgcgcgcgcagctgctggagctgccctacgccaggaaggagctgagcctgctggtgctgctgcctgacgacggcgtggagctcagcacggtggaaaaaagtctcacttttgagaaactcacagcctggaccaagccagactgtatgaagagtactgaggttgaagttctccttccaaaatttaaactacaagaggattatgacatggaatctgtgcttcggcatttgggaattgttgatgccttccaacagggcaaggctgacttgtcggcaatgtcagcggagagagacctgtgtctgtccaagttcgtgcacaagagttttgtggaggtgaatgaagaaggcaccgaggcagcggcagcgtcgagctgctttgtagttgcagagtgctgcatggaatctggccccaggttctgtgctgaccaccctttccttttcttcatcaggcacaacagagccaacagcattctgttctgtggcaggttctcatcgccaggaagcggagctactaacttcagcctgctgaagcaggctggagacgtggaggagaaccctggacctatggactggacctggatcctgttcctggtggccgccgccaccagggtgcacagcggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctagcggcggcggcagcggcggcggcggcagcggcggcggcggcagcggcggcggcggcagcggcggcggcagcctgcagatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccacctatgataaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 42 - protein, 500 aa, synthetic construct
MSRSVALAVLALLSLSGLEAVMAPRTLFLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 43 - protein, 417 aa, synthetic construct
MDWTWILFLVAAATRVHSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHLGSG

SEQ ID NO: 44 - protein, 376 aa, synthetic construct
METLSNASGTFAIRLLKILCQDNPSHNVFCSPVSISSALAMVLLGAKGNTATQMAQALSLNTEEDIHRAFQSLLTEVNKAGTQYLLRTANRLFGEKTCQFLSTFKESCLQFYHAELKELSFIRAAEESRKHINTWVSKKTEGKIEELLPGSSIDAETRLVLVNAIYFKGKWNEPFDETYTREMPFKINQEEQRPVQMMYQEATFKLAHVGEVRAQLLELPYARKELSLLVLLPDDGVELSTVEKSLTFEKLTAWTKPDCMKSTEVEVLLPKFKLQEDYDMESVLRHLGIVDAFQQGKADLSAMSAERDLCLSKFVHKSFVEVNEEGTEAAAASSCFVVAECCMESGPRFCADHPFLFFIRHNRANSILFCGRFSSP

SEQ ID NO: 45 - DNA, 1131 bp, synthetic construct
  source 1..1131: mol_type = other DNA
atggcgcttccggtgacagcactgctcctccccttggcgctgttgctccacgcagcaaggccgcaagtgcagctcgtgcaaagcggcggcggactggtgcaacccggcggatctctgagactgagctgcgccgccagctacttcgacttcgacagctacgagatgagctgggtgaggcaagcccccggaaaaggactggagtggatcggcagcatctaccacagcggctccacctactacaacccctctctgaaatctagagtgaccatctctagagacaactccaagaacacactgtacctccagatgaacacactgagagccgaggacaccgccacctattactgcgctagagtgaacatggacagattcgactactggggccaaggcacactggtgacagtgagctccagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 46 - protein, 377 aa, synthetic construct
MALPVTALLLPLALLLHAARPQVQLVQSGGGLVQPGGSLRLSCAASYFDFDSYEMSWVRQAPGKGLEWIGSIYHSGSTYYNPSLKSRVTISRDNSKNTLYLQMNTLRAEDTATYYCARVNMDRFDYWGQGTLVTVSSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - DNA, 1455 bp, synthetic construct
  source 1..1455: mol_type = other DNA
atggccttaccagtgaccgccttgctcctgccgctggccttgctgctccacgccgccaggccggacgtggtcatgacccagacccctctgagcctgcccgtgagcctgggcgaccaggcttccatctcctgcagaagctcccagagcctggtgcacagcagcggcaacacctacctgcactggtacctgcagaagcccggccagagccccaagctgctgatctacaaggtgtccaacagattttccggcgtgcccgacagattttccggatccggcagcggcaccgacttcacactgaagatcagcagagtggaggccgaggacctgggcgtgtacttctgtagccagagcacacacgtgccttacacctttggcggcggcacaaagctggagatcaagggcggcggcggctccggaggaggaggaagcggaggaggcggctccgaggtgcagctgcagcagtccggccctgacctggtgaagcctggcgcctccatgaagatcagctgcaaggccagcggctactccttcaccgattacaccatgcactgggtgaagcagagccacggcaagaatttcgagtggatcggcctgatcaatccctacaatgacggcaccacctacaatcagaagttcaagggcaaggccacactgacagtggataagagcagctccacagcctacatggagctgctgagcctgacaagcgaggattccgccgtgtactactgcgcctccctggactactggggccagggcacctccgtgaccgtgtcctccaccactaccccagcaccgaggccacccaccccggctcctaccatcgcctcccagcctctgtccctgcgtccggaggcatgtagacccgcagctggtggggccgtgcatacccggggtcttgacttcgcctgcgatttctgggtgctggtcgttgtgggcggcgtgctggcctgctacagcctgctggtgacagtggccttcatcatcttttgggtgaggagcaagcggagcagactgctgcacagcgactacatgaacatgaccccccggaggcctggccccacccggaagcactaccagccctacgcccctcccagggatttcgccgcctaccggagccgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 48 - protein, 484 aa, synthetic construct
MALPVTALLLPLALLLHAARPDVVMTQTPLSLPVSLGDQASISCRSSQSLVHSSGNTYLHWYLQKPGQSPKLLIYKVSNRFSGVPDRFSGSGSGTDFTLKISRVEAEDLGVYFCSQSTHVPYTFGGGTKLEIKGGGGSGGGGSGGGGSEVQLQQSGPDLVKPGASMKISCKASGYSFTDYTMHWVKQSHGKNFEWILINPYNDGTTYNQKFKGKATLTVDKSSSTAYMELLSLTSEDSAVYYCASLDYWGQGTSVTVSSTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - RNA, 20 nt, synthetic construct
  source 1..20: mol_type = other RNA
ggtccatctggtcatagaag

SEQ ID NO: 50 - RNA, 20 nt, synthetic construct
  source 1..20: mol_type = other RNA
gctccaggtagccaccttct

SEQ ID NO: 51 - RNA, 20 nt, synthetic construct
  source 1..20: mol_type = other RNA
taggggccccaactccatgg

SEQ ID NO: 52 - RNA, 20 nt, synthetic construct
  source 1..20: mol_type = other RNA
ggcttatgccaatatcggtg

SEQ ID NO: 53 - RNA, 20 nt, synthetic construct
  source 1..20: mol_type = other RNA
aggtgatgaagagaccaggg

SEQ ID NO: 54 - RNA, 20 nt, synthetic construct
  source 1..20: mol_type = other RNA
ggccgagatgtctcgctccg

SEQ ID NO: 55 - RNA, 20 nt, synthetic construct
  source 1..20: mol_type = other RNA
ggattgctcaacaaccatgc

SEQ ID NO: 56 - RNA, 20 nt, synthetic construct
  source 1..20: mol_type = other RNA
gattgctcaacaaccatgct

SEQ ID NO: 57 - RNA, 20 nt, synthetic construct
  source 1..20: mol_type = other RNA
gtgactgacatcaactccaa

SEQ ID NO: 58 - RNA, 20 nt, synthetic construct
  source 1..20: mol_type = other RNA
cacttgggcattaacacttt

SEQ ID NO: 59 - RNA, 20 nt, synthetic construct
  source 1..20: mol_type = other RNA
ttggaaggcctgcatcatga

SEQ ID NO: 60 - RNA, 20 nt, synthetic construct
  source 1..20: mol_type = other RNA
actccaagggattggaattg

SEQ ID NO: 61 - RNA, 20 nt, synthetic construct
  source 1..20: mol_type = other RNA
gctactctctctttctggcc

SEQ ID NO: 62 - RNA, 20 nt, synthetic construct
  source 1..20: mol_type = other RNA
cgcgagcacagctaaggcca

SEQ ID NO: 63 - RNA, 20 nt, synthetic construct
  source 1..20: mol_type = other RNA
ctagggactgcacagtcaat

SEQ ID NO: 64 - RNA, 20 nt, synthetic construct
  source 1..20: mol_type = other RNA
tcgccgctgccgcggggaca

SEQ ID NO: 65 - RNA, 20 nt, synthetic construct
  source 1..20: mol_type = other RNA
gacatggtcctctgcgttca

SEQ ID NO: 66 - RNA, 20 nt, synthetic construct
  source 1..20: mol_type = other RNA
gtccgctccacagccagcaa

SEQ ID NO: 67 - RNA, 20 nt, synthetic construct
  source 1..20: mol_type = other RNA
gttccagggacggggcccac

SEQ ID NO: 68 - RNA, 20 nt, synthetic construct
  source 1..20: mol_type = other RNA
tcgggcctcgctggccgtaa

SEQ ID NO: 69 - RNA, 20 nt, synthetic construct
  source 1..20: mol_type = other RNA
cgtactaagaacgtgccttc

SEQ ID NO: 70 - RNA, 20 nt, synthetic construct
  source 1..20: mol_type = other RNA
gggttccattacggccagcg

SEQ ID NO: 71 - RNA, 20 nt, synthetic construct
  source 1..20: mol_type = other RNA
caggtgttgtcgggcctcgc

SEQ ID NO: 72 - RNA, 20 nt, synthetic construct
  source 1..20: mol_type = other RNA
tactcaatgcgtacattggt

SEQ ID NO: 73 - RNA, 20 nt, synthetic construct
  source 1..20: mol_type = other RNA
aaggctgaccacatccggaa

SEQ ID NO: 74 - RNA, 20 nt, synthetic construct
  source 1..20: mol_type = other RNA
tacattggtggggccacgag

SEQ ID NO: 75 - RNA, 20 nt, synthetic construct
  source 1..20: mol_type = other RNA
ctgtcagtgaaaaccactcg

SEQ ID NO: 76 - protein, 22 aa, synthetic construct
MLLLVTSLLLCELPHPAFLLIP

SEQ ID NO: 77 - protein, 21 aa, synthetic construct
MALPVTALLLPLALLLHAARP

SEQ ID NO: 78 - protein, 84 aa, synthetic construct
FVPVFLPAKPTTTPAPRPPTPAPTIASQPLSLRPEACRPAAGGAVHTRGLDFACDIYIWAPLAGTCGVLLLSLVITLYCNHRNR

SEQ ID NO: 79 - protein, 23 aa, synthetic construct
IYIWAPLAGTCGVLLLSLVITLY

SEQ ID NO: 80 - protein, 88 aa, synthetic construct
SAAAFVPVFLPAKPTTTPAPRPPTPAPTIASQPLSLRPEACRPAAGGAVHTRGLDFACDIYIWAPLAGTCGVLLLSLVITLYCNHRNR